SEQUENCE LISTING (53 sequences)

SEQ ID NO: 1 - protein, 250 aa, Homo sapiens
MEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 2 - protein, 213 aa, Homo sapiens
MEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATSAEP

SEQ ID NO: 3 - protein, 209 aa, Homo sapiens
MEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEAT

SEQ ID NO: 4 - protein, 233 aa, Homo sapiens
MEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATKSNKYNKGLSPLLSSCHLFVAILK

SEQ ID NO: 5 - protein, 241 aa, Rattus norvegicus
MEKPRGVRCTNGFPERELPRPGASRPAEKSRPPEAKGAQPADAWKAGRPRSEEDNELNLPNLAAAYSSILRSLGEDPQRQGLLKTPWRAATAMQFFTKGYQETISDVLNDAIFDEDHDEMVIVKDIDMFSMCEHHLVPFVGRVHIGYLPNKQVLGLSKLARIVEIYSRRLQVQERLTKQIAVAITEALQPAGVGVVIEATHMCMVMRGVQKMNSKTVTSTMLGVFREDPKTREEFLTLIRS

SEQ ID NO: 6 - protein, 241 aa, Mus musculus
MEKPRGVRCTNGFSERELPRPGASPPAEKSRPPEAKGAQPADAWKAGRHRSEEENQVNLPKLAAAYSSILLSLGEDPQRQGLLKTPWRAATAMQYFTKGYQETISDVLNDAIFDEDHDEMVIVKDIDMFSMCEHHLVPFVGRVHIGYLPNKQVLGLSKLARIVEIYSRRLQVQERLTKQIAVAITEALQPAGVGVVIEATHMCMVMRGVQKMNSKTVTSTMLGVFREDPKTREEFLTLIRS

SEQ ID NO: 7 - protein, 496 aa, Homo sapiens
MPTPDATTPQAKGFRRAVSELDAKQAEAIMSPRFIGRRQSLIEDARKEREAAVAAAAAAVPSEPGDPLEAVAFEEKEGKAVLNLLFSPRATKPSALSRAVKVFETFEAKIHHLETRPAQRPRAGGPHLEYFVRLEVRRGDLAALLSGVRQVSEDVRSPAGPKVPWFPRKVSELDKCHHLVTKFDPDLDLDHPGFSDQVYRQRRKLIAEIAFQYRHGDPIPRVEYTAEEIATWKEVYTTLKGLYATHACGEHLEAFALLERFSGYREDNIPQLEDVSRFLKERTGFQLRPVAGLLSARDFLASLAFRVFQCTQYIRHASSPMHSPEPDCCHELLGHVPMLADRTFAQFSQDIGLASLGASDEEIEKLSTLYWFTVEFGLCKQNGEVKAYGAGLLSSYGELLHCLSEEPEIRAFDPEAAAVQPYQDQTYQSVYFVSESFSDAKDKLRSYASRIQRPFSVKFDPYTLAIDVLDSPQAVRRSLEGVQDELDTLAHALSAI

SEQ ID NO: 8 - protein, 528 aa, Homo sapiens
MPTPDATTPQAKGFRRAVSELDAKQAEAIMVRGQGAPGPSLTGSPWPGTAAPAASYTPTPRSPRFIGRRQSLIEDARKEREAAVAAAAAAVPSEPGDPLEAVAFEEKEGKAVLNLLFSPRATKPSALSRAVKVFETFEAKIHHLETRPAQRPRAGGPHLEYFVRLEVRRGDLAALLSGVRQVSEDVRSPAGPKVPWFPRKVSELDKCHHLVTKFDPDLDLDHPGFSDQVYRQRRKLIAEIAFQYRHGDPIPRVEYTAEEIATWKEVYTTLKGLYATHACGEHLEAFALLERFSGYREDNIPQLEDVSRFLKERTGFQLRPVAGLLSARDFLASLAFRVFQCTQYIRHASSPMHSPEPDCCHELLGHVPMLADRTFAQFSQDIGLASLGASDEEIEKLSTLYWFTVEFGLCKQNGEVKAYGAGLLSSYGELLHCLSEEPEIRAFDPEAAAVQPYQDQTYQSVYFVSESFSDAKDKLRSYASRIQRPFSVKFDPYTLAIDVLDSPQAVRRSLEGVQDELDTLAHALSAIG

SEQ ID NO: 9 - protein, 497 aa, Homo sapiens [sequence omitted]

SEQ ID NO: 10 - protein, 501 aa, Homo sapiens [sequence omitted]

SEQ ID NO: 11 - protein, 528 aa, Homo sapiens; identical to SEQ ID NO: 8

SEQ ID NO: 12 - protein, 338 aa, Artificial Sequence
  Truncated TH corresponding to catalytic domain
MPKVPWFPRKVSELDKCHHLVTKFDPDLDLDHPGFSDQVYRQRRKLIAEIAFQYRHGDPIPRVEYTAEEIATWKEVYTTLKGLYATHACGEHLEAFALLERFSGYREDNIPQLEDVSRFLKERTGFQLRPVAGLLSARDFLASLAFRVFQCTQYIRHASSPMHSPEPDCCHELLGHVPMLADRTFAQFSQDIGLASLGASDEEIEKLSTLYWFTVEFGLCKQNGEVKAYGAGLLSSYGELLHCLSEEPEIRAFDPEAAAVQPYQDQTYQSVYFVSESFSDAKDKLRSYASRIQRPFSVKFDPYTLAIDVLDSPQAVRRSLEGVQDELDTLAHALSAIG

SEQ ID NO: 13 - protein, 497 aa, Artificial Sequence
  Ser40 TH mutant
MPTPDATTPQAKGFRRAVSELDAKQAEAIMSPRFIGRRQELIEDARKEREAAVAAAAAAVPSEPGDPLEAVAFEEKEGKAVLNLLFSPRATKPSALSRAVKVFETFEAKIHHLETRPAQRPRAGGPHLEYFVRLEVRRGDLAALLSGVRQVSEDVRSPAGPKVPWFPRKVSELDKCHHLVTKFDPDLDLDHPGFSDQVYRQRRKLIAEIAFQYRHGDPIPRVEYTAEEIATWKEVYTTLKGLYATHACGEHLEAFALLERFSGYREDNIPQLEDVSRFLKERTGFQLRPVAGLLSARDFLASLAFRVFQCTQYIRHASSPMHSPEPDCCHELLGHVPMLADRTFAQFSQDIGLASLGASDEEIEKLSTLYWFTVEFGLCKQNGEVKAYGAGLLSSYGELLHCLSEEPEIRAFDPEAAAVQPYQDQTYQSVYFVSESFSDAKDKLRSYASRIQRPFSVKFDPYTLAIDVLDSPQAVRRSLEGVQDELDTLAHALSAIG

SEQ ID NO: 14 - protein, 497 aa, Artificial Sequence
  Ser19 Ser40 TH mutant
MPTPDATTPQAKGFRRAVEELDAKQAEAIMSPRFIGRRQELIEDARKEREAAVAAAAAAVPSEPGDPLEAVAFEEKEGKAVLNLLFSPRATKPSALSRAVKVFETFEAKIHHLETRPAQRPRAGGPHLEYFVRLEVRRGDLAALLSGVRQVSEDVRSPAGPKVPWFPRKVSELDKCHHLVTKFDPDLDLDHPGFSDQVYRQRRKLIAEIAFQYRHGDPIPRVEYTAEEIATWKEVYTTLKGLYATHACGEHLEAFALLERFSGYREDNIPQLEDVSRFLKERTGFQLRPVAGLLSARDFLASLAFRVFQCTQYIRHASSPMHSPEPDCCHELLGHVPMLADRTFAQFSQDIGLASLGASDEEIEKLSTLYWFTVEFGLCKQNGEVKAYGAGLLSSYGELLHCLSEEPEIRAFDPEAAAVQPYQDQTYQSVYFVSESFSDAKDKLRSYASRIQRPFSVKFDPYTLAIDVLDSPQAVRRSLEGVQDELDTLAHALSAIG

SEQ ID NO: 15 - protein, 497 aa, Artificial Sequence
  Ser 19 Ser31 Ser40 TH mutant
MPTPDATTPQAKGFRRAVEELDAKQAEAIMEPRFIGRRQELIEDARKEREAAVAAAAAAVPSEPGDPLEAVAFEEKEGKAVLNLLFSPRATKPSALSRAVKVFETFEAKIHHLETRPAQRPRAGGPHLEYFVRLEVRRGDLAALLSGVRQVSEDVRSPAGPKVPWFPRKVSELDKCHHLVTKFDPDLDLDHPGFSDQVYRQRRKLIAEIAFQYRHGDPIPRVEYTAEEIATWKEVYTTLKGLYATHACGEHLEAFALLERFSGYREDNIPQLEDVSRFLKERTGFQLRPVAGLLSARDFLASLAFRVFQCTQYIRHASSPMHSPEPDCCHELLGHVPMLADRTFAQFSQDIGLASLGASDEEIEKLSTLYWFTVEFGLCKQNGEVKAYGAGLLSSYGELLHCLSEEPEIRAFDPEAAAVQPYQDQTYQSVYFVSESFSDAKDKLRSYASRIQRPFSVKFDPYTLAIDVLDSPQAVRRSLEGVQDELDTLAHALSAIG

SEQ ID NO: 16 - protein, 498 aa, Rattus norvegicus
MPTPSAPSPQPKGFRRAVSEQDAKQAEAVTSPRFIGRRQSLIEDARKEREAAAAAAAAAVASSEPGNPLEAVVFEERDGNAVLNLLFSLRGTKPSSLSRAVKVFETFEAKIHHLETRPAQRPLAGSPHLEYFVRFEVPSGDLAALLSSVRRVSDDVRSAREDKVPWFPRKVSELDKCHHLVTKFDPDLDLDHPGFSDQVYRQRRKLIAEIAFQYKHGEPIPHVEYTAEEIATWKEVYVTLKGLYATHACREHLEGFQLLERYCGYREDSIPQLEDVSRFLKERTGFQLRPVAGLLSARDFLASLAFRVFQCTQYIRHASSPMHSPEPDCCHELLGHVPMLADRTFAQFSQDIGLASLGASDEEIEKLSTVYWFTVEFGLCKQNGELKAYGAGLLSSYGELLHSLSEEPEVRAFDPDTAAVQPYQDQTYQPVYFVSESFNDAKDKLRNYASRIQRPFSVKFDPYTLAIDVLDSPHTIQRSLEGVQDELHTLAHALSAIS

SEQ ID NO: 17 - protein, 498 aa, Mus musculus
MPTPSASSPQPKGFRRAVSEQDTKQAEAVTSPRFIGRRQSLIEDARKEREAAAAAAAAAVASAEPGNPLEAVVFEERDGNAVLNLLFSLRGTKPSSLSRALKVFETFEAKIHHLETRPAQRPLAGSPHLEYFVRFEVPSGDLAALLSSVRRVSDDVRSAREDKVPWFPRKVSELDKCHHLVTKFDPDLDLDHPGFSDQAYRQRRKLIAEIAFQYKQGEPIPHVEYTKEEIATWKEVYATLKGLYATHACREHLEAFQLLERYCGYREDSIPQLEDVSHFLKERTGFQLRPVAGLLSARDFLASLAFRVFQCTQYIRHASSPMHSPEPDCCHELLGHVPMLADRTFAQFSQDIGLASLGASDEEIEKLSTVYWFTVEFGLCKQNGELKAYGAGLLSSYGELLHSLSEEPEVRAFDPDTAAVQPYQDQTYQPVYFVSESFSDAKDKLRNYASRIQRPFSVKFDPYTLAIDVLDSPHTIRRSLEGVQDELHTLTQALSAIS

SEQ ID NO: 18 - DNA, 145 bp, adeno-associated virus 2
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 19 - DNA, 145 bp, adeno-associated virus 2
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 20 - DNA, 753 bp, Homo sapiens
atggagaagggccctgtgcgggcaccggcggagaagccgcggggcgccaggtgcagcaatgggttccccgagcgggatccgccgcggcccgggcccagcaggccggcggagaagcccccgcggcccgaggccaagagcgcgcagcccgcggacggctggaagggcgagcggccccgcagcgaggaggataacgagctgaacctccctaacctggcagccgcctactcgtccatcctgagctcgctgggcgagaacccccagcggcaagggctgctcaagacgccctggagggcggcctcggccatgcagttcttcaccaagggctaccaggagaccatctcagatgtcctaaacgatgctatatttgatgaagatcatgatgagatggtgattgtgaaggacatagacatgttttccatgtgtgagcatcacttggttccatttgttggaaaggtccatattggttatcttcctaacaagcaagtccttggcctcagcaaacttgcgaggattgtagaaatctatagtagaagactacaagttcaggagcgccttacaaaacaaattgctgtagcaatcacggaagccttgcggcctgctggagtcggggtagtggttgaagcaacacacatgtgtatggtaatgcgaggtgtacagaaaatgaacagcaaaactgtgaccagcacaatgttgggtgtgttccgggaggatccaaagactcgggaagagttcctgactctcattaggagctaa

SEQ ID NO: 21 - DNA, 155 bp, Simian virus 40
ttcgagcaacttgtttattgcagcttataatggttacaaataaagcaatagcatcacaaatttcacaaataaagcatttttttcactgcattctagttgtggtttgtccaaactcatcaatgtatcttatcatgtctggatcgtctagcatcgaa

SEQ ID NO: 22 - DNA, 192 bp, Simian virus 40
cagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggtttttt

SEQ ID NO: 23 - DNA, 1490 bp, Homo sapiens
atgcccacccccgacgccaccacgccacaggccaagggcttccgcagggccgtgtctgagctggacgccaagcaggcagaggccatcatgtccccgcggttcattgggcgcaggcagagcctcatcgaggacgcccgcaaggagcgggaggcggcggtggcagcagcggccgctgcagtcccctcggagcccggggaccccctggaggctgtggcctttgaggagaaggaggggaaggccgtgctaaacctgctcttctccccgagggccaccaagccctcggcgctgtcccgagctgtgaaggtgtttgagacgtttgaagccaaaatccaccatctagagacccggcccgcccagaggccgcgagctgggggcccccacctggagtacttcgtgcgcctcgaggtgcgccgaggggacctggccgccctgctcagtggtgtgcgccaggtgtcagaggacgtgcgcagccccgcggggcccaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtactggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattgg

SEQ ID NO: 24 - DNA, 1029 bp, Homo sapiens
atgagccccgcggggcccaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtactggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattggctaa

SEQ ID NO: 25 - DNA, 1491 bp, Artificial Sequence
  Ser40 TH mutant
  misc_feature (1)..(1491): Ser40 TH mutant
atgcccacccccgacgccaccacgccacaggccaagggcttccgcagggccgtgtctgagctggacgccaagcaggcagaggccatcatgtccccgcggttcattgggcgcaggcaggagctcatcgaggacgcccgcaaggagcgggaggcggcggtggcagcagcggccgctgcagtcccctcggagcccggggaccccctggaggctgtggcctttgaggagaaggaggggaaggccgtgctaaacctgctcttctccccgagggccaccaagccctcggcgctgtcccgagctgtgaaggtgtttgagacgtttgaagccaaaatccaccatctagagacccggcccgcccagaggccgcgagctgggggcccccacctggagtacttcgtgcgcctcgaggtgcgccgaggggacctggccgccctgctcagtggtgtgcgccaggtgtcagaggacgtgcgcagccccgcggggcccaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtactggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattggc

SEQ ID NO: 26 - DNA, 1491 bp, Artificial Sequence
  Ser19 Ser49 TH mutant
  misc_feature (1)..(1491): Ser19, Ser40 TH mutant
atgcccacccccgacgccaccacgccacaggccaagggcttccgcagggccgtggaggagctggacgccaagcaggcagaggccatcatgtccccgcggttcattgggcgcaggcaggagctcatcgaggacgcccgcaaggagcgggaggcggcggtggcagcagcggccgctgcagtcccctcggagcccggggaccccctggaggctgtggcctttgaggagaaggaggggaaggccgtgctaaacctgctcttctccccgagggccaccaagccctcggcgctgtcccgagctgtgaaggtgtttgagacgtttgaagccaaaatccaccatctagagacccggcccgcccagaggccgcgagctgggggcccccacctggagtacttcgtgcgcctcgaggtgcgccgaggggacctggccgccctgctcagtggtgtgcgccaggtgtcagaggacgtgcgcagccccgcggggcccaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtactggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattggc

SEQ ID NO: 27 - DNA, 1491 bp, Artificial Sequence
  Ser19 Ser31 Ser40 TH mutant
  misc_feature (1)..(1491): Ser19, Ser31, Ser40 TH mutant
atgcccacccccgacgccaccacgccacaggccaagggcttccgcagggccgtggaggagctggacgccaagcaggcagaggccatcatggagccgcggttcattgggcgcaggcaggagctcatcgaggacgcccgcaaggagcgggaggcggcggtggcagcagcggccgctgcagtcccctcggagcccggggaccccctggaggctgtggcctttgaggagaaggaggggaaggccgtgctaaacctgctcttctccccgagggccaccaagccctcggcgctgtcccgagctgtgaaggtgtttgagacgtttgaagccaaaatccaccatctagagacccggcccgcccagaggccgcgagctgggggcccccacctggagtacttcgtgcgcctcgaggtgcgccgaggggacctggccgccctgctcagtggtgtgcgccaggtgtcagaggacgtgcgcagccccgcggggcccaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtactggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattggc

SEQ ID NO: 28 - DNA, 610 bp, Woodchuck hepatitis B virus
cgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagct

SEQ ID NO: 29 - DNA, 610 bp, Artificial Sequence
  Mutated Woodchuck hepatitis B virus (WHV8) post-transcriptional regulatory element
  misc_feature (1)..(610): Mutated WHV8
cgtcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagct

SEQ ID NO: 30 - DNA, 748 bp, Homo sapiens
atggagaagggccctgtgcgggcaccggcggagaagccgcggggcgccaggtgcagcaatgggttccccgagcgggatccgccgcggcccgggcccagcaggccggcggagaagcccccgcggcccgaggccaagagcgcgcagcccgcggacggctggaagggcgagcggccccgcagcgaggaggataacgagctgaacctccctaacctggcagccgcctactcgtccatcctgagctcgctgggcgagaacccccagcggcaagggctgctcaagacgccctggagggcggcctcggccatgcagttcttcaccaagggctaccaggagaccatctcagatgtcctaaacgatgctatatttgatgaagatcatgatgagatggtgattgtgaaggacatagacatgttttccatgtgtgagcatcacttggttccatttgttggaaaggtccatattggttatcttcctaacaagcaagtccttggcctcagcaaacttgcgaggattgtagaaatctatagtagaagactacaagttcaggagcgccttacaaaacaaattgctgtagcaatcacggaagccttgcggcctgctggagtcggggtagtggttgaagcaacacacatgtgtatggtaatgcgaggtgtacagaaaatgaacagcaaaactgtgaccagcacaatgttgggtgtgttccgggaggatccaaagactcgggaagagttcctgactctcattagga

SEQ ID NO: 31 - DNA, 4205 bp, Artificial Sequence
  pAA011 - scAAV-HLP-GCH1
  misc_feature (1)..(4205): pAA011 - scAAV-HLP-GCH1
aaagcttcccggggggatctgggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttccttctagatgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgctagcgaattctagcatggagaagggccctgtgcgggcaccggcggagaagccgcggggcgccaggtgcagcaatgggttccccgagcgggatccgccgcggcccgggcccagcaggccggcggagaagcccccgcggcccgaggccaagagcgcgcagcccgcggacggctggaagggcgagcggccccgcagcgaggaggataacgagctgaacctccctaacctggcagccgcctactcgtccatcctgagctcgctgggcgagaacccccagcggcaagggctgctcaagacgccctggagggcggcctcggccatgcagttcttcaccaagggctaccaggagaccatctcagatgtcctaaacgatgctatatttgatgaagatcatgatgagatggtgattgtgaaggacatagacatgttttccatgtgtgagcatcacttggttccatttgttggaaaggtccatattggttatcttcctaacaagcaagtccttggcctcagcaaacttgcgaggattgtagaaatctatagtagaagactacaagttcaggagcgccttacaaaacaaattgctgtagcaatcacggaagccttgcggcctgctggagtcggggtagtggttgaagcaacacacatgtgtatggtaatgcgaggtgtacagaaaatgaacagcaaaactgtgaccagcacaatgttgggtgtgttccgggaggatccaaagactcgggaagagttcctgactctcattaggagctaatgcatccccatcgatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgaccgctagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctag

SEQ ID NO: 32 - DNA, 5129 bp, Artificial Sequence
  pAA016 - scAAV-HLP-tTH
  misc_feature (1)..(5129): pAA016 - scAAV-HLP-tTH
aaagcttcccggggggatctgggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacccctaaaatgggcaaacattgcgctagctgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcattcaagctcgtagcaaggatccaccggtcaccatgagccccgcggggcccaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtactggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattggctaactagtggatccgtcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacagcttatcgataccgtcgacttcgagcaacttgtttattgcagcttataatggttacaaataaagcaatagcatcacaaatttcacaaataaagcatttttttcactgcattctagttgtggtttgtccaaactcatcaatgtatcttatcatgtctggatcgtctagcatcgaagatcccccgctagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctag

SEQ ID NO: 33 - DNA, 8013 bp, Artificial Sequence
  pAAo19 - scAAV-LP1-GCH1-LP1-tTH
  misc_feature (1)..(8013): pAAo19 - scAAV-LP1-GCH1-LP1-tTH
cagcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagagctgagcccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctcttttagattccaacctttggaactgacctgcaggattcaagctgctagcaaggatccaccggtaacatggagaagggccctgtgcgggcaccggcggagaagccgcggggcgccaggtgcagcaatgggttccccgagcgggatccgccgcggcccgggcccagcaggccggcggagaagcccccgcggcccgaggccaagagcgcgcagcccgcggacggctggaagggcgagcggccccgcagcgaggaggataacgagctgaacctccctaacctggcagccgcctactcgtccatcctgagctcgctgggcgagaacccccagcggcaagggctgctcaagacgccctggagggcggcctcggccatgcagttcttcaccaagggctaccaggagaccatctcagatgtcctaaacgatgctatatttgatgaagatcatgatgagatggtgattgtgaaggacatagacatgttttccatgtgtgagcatcacttggttccatttgttggaaaggtccatattggttatcttcctaacaagcaagtccttggcctcagcaaacttgcgaggattgtagaaatctatagtagaagactacaagttcaggagcgccttacaaaacaaattgctgtagcaatcacggaagccttgcggcctgctggagtcggggtagtggttgaagcaacacacatgtgtatggtaatgcgaggtgtacagaaaatgaacagcaaaactgtgaccagcacaatgttgggtgtgttccgggaggatccaaagactcgggaagagttcctgactctcattaggagctaatgcatccccatcgatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgaccagatctgacaaggtcccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctcttttagattccaacctttggaactgattcgaaattcaagctgctagcaaggatccaccggtcaccatgagccccgcggggcccaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtactggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattggctaactagtggatccgtcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacagcttatcgataccgtcgacttcgagcaacttgtttattgcagcttataatggttacaaataaagcaatagcatcacaaatttcacaaataaagcatttttttcactgcattctagttgtggtttgtccaaactcatcaatgtatcttatcatgtctggatcgtctagcatcgaagatcccccgcatgctctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgc

SEQ ID NO: 34 - DNA, 5369 bp, Artificial Sequence
  pAA010 scAAV-LP1-tTH
  misc_feature (1)..(5369): pAA010 scAAV-LP1-tTH
aaagcttcccggggggatctgggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctcttttagattccaacctttggaactgaattctagcatgagccccgcggggcccaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtactggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattggctaactagtggatccgtcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacagcttatcgataccgtcgacttcgagcaacttgtttattgcagcttataatggttacaaataaagcaatagcatcacaaatttcacaaataaagcatttttttcactgcattctagttgtggtttgtccaaactcatcaatgtatcttatcatgtctggatcgtctagcatcgaagatcccccgctagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctag

SEQ ID NO: 35 - DNA, 4503 bp, Artificial Sequence
  pAA009 scAAV-LP1-GCH1
  misc_feature (1)..(4503): pAA009 scAAV-LP1-GCH1
aaagcttcccggggggatctgggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctcttttagattccaacctttggaactgaattctagcatggagaagggccctgtgcgggcaccggcggagaagccgcggggcgccaggtgcagcaatgggttccccgagcgggatccgccgcggcccgggcccagcaggccggcggagaagcccccgcggcccgaggccaagagcgcgcagcccgcggacggctggaagggcgagcggccccgcagcgaggaggataacgagctgaacctccctaacctggcagccgcctactcgtccatcctgagctcgctgggcgagaacccccagcggcaagggctgctcaagacgccctggagggcggcctcggccatgcagttcttcaccaagggctaccaggagaccatctcagatgtcctaaacgatgctatatttgatgaagatcatgatgagatggtgattgtgaaggacatagacatgttttccatgtgtgagcatcacttggttccatttgttggaaaggtccatattggttatcttcctaacaagcaagtccttggcctcagcaaacttgcgaggattgtagaaatctatagtagaagactacaagttcaggagcgccttacaaaacaaattgctgtagcaatcacggaagccttgcggcctgctggagtcggggtagtggttgaagcaacacacatgtgtatggtaatgcgaggtgtacagaaaatgaacagcaaaactgtgaccagcacaatgttgggtgtgttccgggaggatccaaagactcgggaagagttcctgactctcattaggagctaatgcatccccatcgatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgaccgctagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctag

SEQ ID NO: 36 - DNA, 5059 bp, Artificial Sequence
  scAAV-LP1-hFIXco
  misc_feature (1)..(5059): scAAV-LP1-hFIXco
aaagcttcccggggggatctgggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctcttttagattccaacctttggaactgaattctagaccaccatgcagagggtgaacatgatcatggctgagagccctggcctgatcaccatctgcctgctgggctacctgctgtctgctgagtgcactgtgttcctggaccatgagaatgccaacaagatcctgaacaggcccaagagatacaactctggcaagctggaggagtttgtgcagggcaacctggagagggagtgcatggaggagaagtgcagctttgaggaggccagggaggtgtttgagaacactgagaggaccactgagttctggaagcagtatgtggatggggaccagtgtgagagcaacccctgcctgaatgggggcagctgcaaggatgacatcaacagctatgagtgctggtgcccctttggctttgagggcaagaactgtgagctggatgtgacctgcaacatcaagaatggcagatgtgagcagttctgcaagaactctgctgacaacaaggtggtgtgcagctgcactgagggctacaggctggctgagaaccagaagagctgtgagcctgctgtgccattcccatgtggcagagtgtctgtgagccagaccagcaagctgaccagggctgaggctgtgttccctgatgtggactatgtgaacagcactgaggctgaaaccatcctggacaacatcacccagagcacccagagcttcaatgacttcaccagggtggtggggggggaggatgccaagcctggccagttcccctggcaagtggtgctgaatggcaaggtggatgccttctgtgggggcagcattgtgaatgagaagtggattgtgactgctgcccactgtgtggagactggggtgaagatcactgtggtggctggggagcacaacattgaggagactgagcacactgagcagaagaggaatgtgatcaggatcatcccccaccacaactacaatgctgccatcaacaagtacaaccatgacattgccctgctggagctggatgagcccctggtgctgaacagctatgtgacccccatctgcattgctgacaaggagtacaccaacatcttcctgaagtttggctctggctatgtgtctggctggggcagggtgttccacaagggcaggtctgccctggtgctgcagtacctgagggtgcccctggtggacagggccacctgcctgaggagcaccaagttcaccatctacaacaacatgttctgtgctggcttccatgaggggggcagggacagctgccagggggactctgggggcccccatgtgactgaggtggagggcaccagcttcctgactggcatcatcagctggggggaggagtgtgccatgaagggcaagtatggcatctacaccaaagtctccagatatgtgaactggatcaaggagaagaccaagctgacctgactcgatgctttatttgtgaaatttgtgatgctattgctttatttgtaaccattataagctgcaataaacaagttaacaacaacaattgcattcattttatgtttcaggttcagggggaggtgtgggaggttttttaaactagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctag

SEQ ID NO: 37 - DNA, 9189 bp, Artificial Sequence
  pAV HLP FVIII V3 kan
  misc_feature (1)..(9189): pAV HLP FVIII V3 kan
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttcccggggggatctttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccggaggggtggagtcgtgacgtgaattacgtcatagggttagggaggtcgtatactgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgcggccgccaccatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaatgccactaatgtgtctaacaacagcaacaccagcaatgacagcaatgtgtctcccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgatcgcgaataaaagatctttattttcattagatctgtgtgttggttttttgtgtgatgcagcccaagctgtagataagtagcatggcgggttaatcattaactacacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaaagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaattcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 38 - DNA, 252 bp, Artificial Sequence
  Hybrid liver-specific promoter (HLP)
  misc_feature (1)..(252): Hybrid liver-specific promoter (HLP)
tgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatc

SEQ ID NO: 39 - DNA, 545 bp, Homo sapiens
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctcttttagattccaacctttggaactga

SEQ ID NO: 40 - protein, 342 aa, Artificial Sequence
  tTH = truncated Tyrosine Hydroxylase
  PEPTIDE (1)..(342): tTH = truncated Tyrosine Hydroxylase
MSPAGPKVPWFPRKVSELDKCHHLVTKFDPDLDLDHPGFSDQVYRQRRKLIAEIAFQYRHGDPIPRVEYTAEEIATWKEVYTTLKGLYATHACGEHLEAFALLERFSGYREDNIPQLEDVSRFLKERTGFQLRPVAGLLSARDFLASLAFRVFQCTQYIRHASSPMHSPEPDCCHELLGHVPMLADRTFAQFSQDIGLASLGASDEEIEKLSTLYWFTVEFGLCKQNGEVKAYGAGLLSSYGELLHCLSEEPEIRAFDPEAAAVQPYQDQTYQSVYFVSESFSDAKDKLRSYASRIQRPFSVKFDPYTLAIDVLDSPQAVRRSLEGVQDELDTLAHALSAIG

SEQ ID NO: 41 - DNA, 438 bp, Homo sapiens
atgagcacggaaggtggtggccgtcgctgccaggcacaagtgtcccgccgcatctccttcagcgcgagccaccgattgtacagtaaatttctaagtgatgaagaaaacttgaaactgtttgggaaatgcaacaatccaaatggccatgggcacaattataaagttgtggtgacagtacatggagagattgaccctgctacgggaatggttatgaatctggctgatctcaaaaaatatatggaggaggcgattatgcagccccttgatcataagaatctggatatggatgtgccatactttgcagatgtggtgagcacgactgaaaatgtagctgtttatatctgggacaacctccagaaagttcttcctgtaggagttctttataaagtaaaagtatacgaaactgacaataatattgtggtttataaaggagaatag

SEQ ID NO: 42 - DNA, 28 bp, Artificial Sequence
  primer AA16
  misc_feature (1)..(28): primer AA16
ccaagctagcatggagaagggccctgtg

SEQ ID NO: 43 - DNA, 29 bp, Artificial Sequence
  primer AA17
  misc_feature (1)..(29): primer AA17
ccaagctagcggtcgactaaaaaacctcc

SEQ ID NO: 44 - DNA, 31 bp, Artificial Sequence
  primer AA33
  misc_feature (1)..(31): primer AA33
ccaagctagcatgagccccgcggggcccaag

SEQ ID NO: 45 - DNA, 31 bp, Artificial Sequence
  primer AA34
  misc_feature (1)..(21): primer AA34
ccaagctagcgggggatcttcgatgctagac

SEQ ID NO: 46 - DNA, 60 bp, Artificial Sequence
  primer AA43
  misc_feature (1)..(60): primer AA43
ccaatggccaactccatcactaggggttccttctagatgtttgctgcttgcaatgtttgc

SEQ ID NO: 47 - DNA, 38 bp, Artificial Sequence
  primer AA44
  misc_feature (1)..(38): primer AA44
ccaagaattcgctagcgattcactgtcccaggtcagtg

SEQ ID NO: 48 - DNA, 33 bp, Artificial Sequence
  primer AA57
  misc_feature (1)..(33): primer AA57
ccaagctagctgtttgctgcttgcaatgtttgc

SEQ ID NO: 49 - DNA, 43 bp, Artificial Sequence
  primer AA67
  misc_feature (1)..(43): primer AA67
gatccttgctacgagcttgaatgattcactgtcccaggtcagt

SEQ ID NO: 50 - DNA, 43 bp, Artificial Sequence
  Primer AA68
  misc_feature (1)..(43): Primer AA68
actgacctgggacagtgaatcattcaagctcgtagcaaggatc

SEQ ID NO: 51 - DNA, 29 bp, Artificial Sequence
  primer RmiscTHext2
  misc_feature (1)..(29): primer RmiscTHext2
aaagctagcttcgatgctagacgatccag

SEQ ID NO: 52 - DNA, 5638 bp, Artificial Sequence
  Monocistronic delivery plasmid TH
  misc_feature (1)..(5638): Monocistronic delivery plasmid TH
gcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagctagagatcccgggaccatgagccccgcggggcccaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtactggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattggctaagacgccacctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttcccatcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaactcgactctggctctatcgaatctccgtcgtttcgagcttacgcgaacagccgtggcgctcatttgctcgtcgggcatcgaatctcgtcagctatcgtcagcttacctttttggca

SEQ ID NO: 53 - DNA, 6431 bp, Artificial Sequence
  Bicistronic delivery plasmid GCH1 PTPS
  misc_feature (1)..(6431): Bicistronic delivery plasmid GCH1 PTPS
gcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcaatatggccacaaccgcggccgtagatcccgggaccatggagaagccgcggggagtcaggtgcaccaatgggttctccgagcgggagctgccgcggcccggggccagcccgcctgccgagaagtcccggccgcccgaggccaagggcgcacagccggccgacgcctggaaggcagggcggcaccgcagcgaggaggaaaaccaggtgaacctccccaaactggcggctgcttactcgtccattctgctctcgctgggcgaggacccccagcggcaggggctgctcaagacgccctggagggcggccaccgccatgcagtacttcaccaagggataccaggagaccatctcagatgtcctgaatgatgctatatttgatgaagatcatgacgagatggtgattgtgaaggacatagatatgttctccatgtgtgagcatcaccttgttccatttgtaggaagggtccatattggctatcttcctaacaagcaagtccttggtctcagtaaacttgccaggattgtagaaatctacagtagacgactacaagttcaagagcgcctcaccaaacagattgcggtggccatcacagaagccttgcagcctgctggcgttggagtagtgattgaagcgacacacatgtgcatggtaatgcgaggcgtgcagaaaatgaacagcaagactgtcactagcaccatgctgggcgtgttccgggaagaccccaagactcgggaggagttcctcacactaatcaggagctgagactatagggtgggtattatgtgttcatcaaccatcctaaaaatacccggtaaacaggtgcagccccagatctgggcagcaggagggggcagtgggaagcttaacgcgccacgactatagggtgggtattatgtgttcatcaaccatcctaaaaatacccggtaaacaggtgcagccccagatctgggcagcaggagggggcagtgggaagcttatctagtctcgaggtaccgagctcttacgcgtgctagctcgagatctggatatcgactatagggtgggtattatgtgttcatcaaccatcctaaaaatacccggtaaacaggtgcagccccagatctgggcagcaggagggggcagtgggtctgttctatttttaccagccagttgctgctggacacagttttcatagcctcccctcggctctgcccctcacagtctgcagtctacggcgaggcacaggccagcccagctccacgaggactgaacaagaagcttgatatcgaattggtaccatcgaggaactgaaaaaccagaaagttaactggtaagtttagtctttttgtcttttatttcaggtcccggatccggtggtggtgcaaatcaaagaactgctcctcagtggatatcgcctttacttctaggccaccatgagcgcggcgggtgaccttcgtcgccgcgcgcgactgtcgcgcctcgtgtccttcagcgcgagccaccggctgcacagcccatctctgagcgatgaagagaacttaagagtgtttgggaaatgcaacaatccgaatggccacgggcacaactataaagttgtggtgacagtccatggagagattgatcctgttacaggaatggttatgaatttgaccgacctcaaagaatacatggaggaggccatcatgaagcctcttgatcacaagaacctggacctggatgtgccgtactttgcggatgctgtgagcacgacagaaaatgtagctgtctacatctgggaaagcctccagaaacttcttccagtgggagctctttataaagtaaaagtgtttgaaaccgacaacaacatcgtagtctataaaggagaatagtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttcccatcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaactcgactctggctctatcgaatctccgtcgtttcgagcttacgcgaacagccgtggcgctcatttgctcgtcgggcatcgaatctcgtcagctatcgtcagcttacctttttggca

(19) United States
(12) Patent Application Publication
(10) Pub. No.: US 2019/0032079 A1
(43) Pub. Date: Jan. 31, 2019

(54) SYSTEMIC SYNTHESIS AND REGULATION OF L-DOPA

(71) Applicant: MYODOPA LIMITED, Guildford, Surrey (GB)

(72) Inventor: Michael McDonald, Guildford, Surrey (GB)

(21) Appl. No.: 15/748,145

(22) PCT Filed: Aug. 1, 2016
(86) PCT No.: PCT/EP2016/068315

§ 371 Date: Jan. 26, 2018

Related U.S. Application Data
(60) Provisional application No. 62/200,151, filed on Aug. 3, 2015.

(51) Int. Cl. C12N 15/86; A61P 25/16; A61K 45/06; A61K 31/519; A61K 9/00; A61K 31/713; C12N 9/78; C12N 9/02; C12N 9/88

(52) U.S. Cl. CPC C12N 15/86; A61P 25/16; A61K 45/06; A61K 31/519; A61K 9/0019; A61K 31/713; C12N 2750/14171; C12Y 305/04016; C12N 9/0071; C12Y 114/16002; C12N 9/88; C12Y 402/03012; C12N 2750/14143; C12N 9/78

(57) ABSTRACT

The present invention relates to an expression system for enzyme replacement therapy with the aim of obtaining or maintaining a steady level of L-DOPA in the blood of an individual, achieved through systemic administration of the expression system. The invention is thus useful in the treatment of catecholamine deficient disorders, such as dopamine deficient disorders including Parkinson's Disease.

All patent and non-patent references cited in the present application, are hereby incorporated by reference in their entirety.

### I. TECHNICAL FIELD OF THE INVENTION

The present invention relates to expression systems comprising polynucleotide sequences encoding polypeptides to be differentially expressed in a target cell; and administered peripherally to a patient in need thereof for treating medical conditions associated with catecholamine dysfunction, in particular diseases associated with dopamine deficiency such as Parkinson's disease and related disorders including L-DOPA induced dyskinesia.

### II. BACKGROUND OF THE INVENTION

Parkinson's disease (PD) is a common neurodegenerative disease characterized clinically by resting tremor, rigidity, slowness of voluntary movement, and postural instability. Loss of dopaminergic neurons within the substantia nigra pars compacta (SNpc), intraneuronal cytoplasmic inclusions or “Lewy bodies,” gliosis, and striatal dopamine depletion are principal neuropathological findings. With the exception of inherited cases linked to specific gene defects that account for 10% of cases, PD is a sporadic condition of unknown cause.

Dopamine does not cross the blood brain barrier. Striatal dopamine deletion cannot be resolved by peripheral administration of dopamine. Therapy with the dopamine (DA) precursor L-3,4-dihydroxyphenylalanine (L-DOPA) is the most effective treatment for Parkinson's disease. However, while treatment response is excellent initially, over the course of several years most patients develop therapy-related adverse effects such as L-DOPA-induced dyskinesias. (Obeso, Olanow, & Nutt, 2000) (Ahlskog & Muenter, 2001). These complications are thought to arise from the intermittent and pulsatile stimulation of supersensitive DA receptors on striatal neurons. (Chase, 1998) (Nutt, Obeso, & Stocchi, 2000)

Nigral dopamine neurons fire tonically at a steady rate of ˜4 cycles/second. This background firing is interrupted briefly by phasic bursts upon presentation of an unexpected or rewarding stimulus such as food. Since the amount of neurotransmitter release generally reflects the rate of neuronal firing, striatal dopamine concentrations remain within a fairly narrow range, and dopamine receptors at the nigrostriatal synapses are exposed to fairly stable concentrations of their cognate neurotransmitter. As denervation of the nigrostriatal dopaminergic neurons increases, exposure to striatal dopamine formed from exogenous dopa becomes increasingly brief, and the relative rise and fall of dopamine concentrations acquires an amplitude that is larger than the amplitude that occurs physiologically. In early disease, the inevitable variability in the delivery of dopa consequent upon oral administration goes largely unnoticed, and most patients experience sustained benefit. This stable response reflects the capacity of residual dopaminergic neurons to transform exogenous dopa into a long-duration motor response. These observations are consistent with the notion that the presence of an adequate surviving complement of nigral dopaminergic neurons in early Parkinson's disease shields the striatum from the vicissitudes of brain dopa.

As treatment continues, the pharmacokinetic properties of L-DOPA start to assume greater clinical relevance, and a shorter-duration motor response predicted from the 90-minute half-life becomes apparent.

Continuous DA receptor stimulation using either duodenal (Syed, Murphy, Zimmerman, Mark, & Sage, 1998) (Nyholm et al., 2003) or intravenous (Mouradian, Heuser, Baronti, & Chase, 1990) infusion of L-DOPA, or subcutaneous infusion of the DA receptor agonist apomorphine (Poewe & Wenning, 2000) has been shown to markedly reduce the frequency and severity of abnormal involuntary movements in Parkinson's disease patients

Continuous delivery of a gel formulation of levodopa/carbidopa into the duodenum via a percutaneous tube and a portable pump provides more constant plasma concentrations than oral drug therapy. The therapy (Duodopa) has been approved in the USA and in the EU under an orphan drug exemption and is currently used in ˜800 patients. The evidence base for this therapy is still evolving. Nyholm conducted a randomized crossover study and proved superiority of duodenal levodopa infusion over oral polypharmacy in reducing off periods and on time with severe dyskinesia. (Nyholm et al., 2005) This symptomatic benefit has been confirmed in open-label studies (Nilsson, Nyholm, & Aquilonius, 2001), (Nyholm et al., 2008). More recently, (Antonini, Chaudhuri, Martinez-Martin, & Odin, 2010) evaluated prospectively the longer-term impact of the therapy on health-related quality of life in nine patients with advanced Parkinson's disease. The therapy significantly shortened the daily duration of off periods and dyskinesia. This led to significant improvements in four domains (mobility, ADL, stigma, and bodily discomfort) of the PDQ-39. (Wolters, Lees, Volkmann, van Laar, & Hovestadt, 2008)

A pharmacokinetic-pharmacodynamic study of duodopa for PD indicated a concentration at 50% effect of 1.55 mg/L L-Dopa (Westin et al., 2011). A similar study using an intra-intestinal infusion of levodopa methyl ester achieved improved control of PD and dyskinesia with plasma levels of 3000-4000 ng/mL of Levodopa.

Direct injection of viral vectors in the parkinsonian brain provides a continuous and local production of L-DOPA centrally at a specific target site in the brain, i.e. in the DA-depleted striatum. Local L-DOPA delivery by in vivo gene therapy, using intrastriatal gene transfer of DA-synthetic enzyme tyrosine hydroxylase (TH), has been explored as a potential therapeutic intervention for Parkinson's disease (Horellou et al., 1994) (Kaplitt et al., 1994). It has been shown that the levels of DOPA production are very low unless expression of TH is combined with exogenous administration of tetrahydrobiopterin, the co-factor for TH, or with co-expression of its rate-limiting synthetic enzyme, GTP cyclohydrolase 1 (GCH1) (Mandel, Spratt, Snyder, & Leff, 1997) (Bencsics et al., 1996) (Corti et al., 1999). The most promising long-term results so far have been obtained using recombinant adeno-associated viral (rAAV) vectors (Mandel et al., 1998) (Kink, Rosenblad, & Bjorklund, 1998), (Szczypka et al., 1999). It has been shown that intrastriatal injection of high titre rAAV vectors encoding the genes for TH and GCH1 can provide pronounced behavioural recovery in rats rendered parkinsonian by injection of 6-hydroxydopamine (6-OHDA), provided that the level of striatal DOPA production exceeds a critical threshold (Kink et al., 2002). Further study indicated that rAAV-mediated expression of the DOPA-synthesizing enzymes, TH and GCH1, in the striatum is capable of eliminating L-DOPA-induced dyskinesias in the rat Parkinson's disease model. In vivo gene therapy by rAAV-TH and rAAV-GCH1 vectors has dual action: (i) alleviation of dyskinesias induced by systemic intermittent L-DOPA treatment; and (ii) near complete reversal of the lesion-induced deficits in spontaneous motor behaviour. These changes are associated with a normalization of striatal opioid gene expression and reversal of the abnormal DFosB expression, both of which are considered as markers of maladaptive plasticity induced by the L-DOPA treatment. (Carlsson et al., 2005).

An improved treatment for Parkinson's disease would enable long term constant administration of L-DOPA by a route which did not require interventional brain surgery, life-long intravenous infusion or require surgical implantation of a percutaneous endoscopic gastrostomy tube with the risks and complications associated with each route of administration.

While direct production at the site of intended use has a number of advantages (minimal dose requirement and lack of peripheral effects) the route of administration requires neurosurgery. The requirement of intrastriatal injection is likely to limit clinical application to a subset of patients expected to benefit from the intervention. There are at present insufficient neurosurgical facilities and neurosurgeons to ensure that all eligible patients could be treated by such methods.

### III. SUMMARY OF THE INVENTION

Direct continuous secretion of a therapeutic or sub-therapeutic level of L-DOPA into the peripheral circulation would circumvent problems associated with enteral administration including unwanted decarboxylation in the gut and inconsistent absorption due to ingested food, Helicobacter pylori infection, variations in gut motility and gastric acidity, competition for absorption across the gut wall from dietary neutral amino acids, and DOPA metabolites formed by gut flora.

While direct continuous secretion into the vascular system of a therapeutic level of L-DOPA might be optimal, continuous secretion of sub-therapeutic level may still be valuable, thus facilitating sufficient constant background levels of striatal dopamine to prevent or delay the development of dyskinesia and minimising the dose of oral L-DOPA supplements needed for efficacy.

Rather than to continuously infuse L-DOPA via the gut or parenterally it is proposed to enable one or more peripheral tissues such as liver or muscle to continuously secrete L-DOPA into the peripheral circulation. This is achieved by introducing into the target tissues the genes to enable L-DOPA. Tyrosine hydroxylase (TH) catalyzes the hydroxylation of tyrosine to L-DOPA and needs tetrahydrobiopterin (BH4) as cofactor. BH4 biosynthesis may require the GTP cyclohydrolase 1 (GCH1).

Secretion of levels of L-DOPA into the peripheral circulation will reduce the requirement for other forms of dopaminergic therapy such as oral L-DOPA or dopamine agonists in conditions due to dopamine deficiency such as Parkinson's disease. Optimal levels of L-DOPA secretion would remove the need for additional dopamine agonist(s). Even less than optimal levels of L-DOPA secretion would reduce the dose of additional agonist(s). This could reduce the adverse events associated with use of oral or parenteral L-DOPA or dopamine agonists or other treatments for dopamine deficiency.

Troublesome complications of oral and parenteral L-DOPA therapy and dopaminergic agonists such as L-DOPA induced dyskinesia and on/off syndrome are believed due to the fluctuations in the pharmacokinetic peak and trough levels of these agents following oral or parenteral dosing. Achieving constant secretion of L-DOPA into the peripheral circulation at therapeutic or sub-therapeutic levels would establish a raised baseline level of plasma L-DOPA and facilitate reduction of the dose of additional dopaminergic agents thus reducing peak to trough variation.

The purpose of the present invention has been to develop new molecular tools for the treatment of disorders where the present treatment strategies are insufficient or where present treatment is associated with severe side effects and/or where the treated individual develops resistance against said treatment. More specifically, the present invention relates to a novel expression construct regulating the level of enzymes involved in catecholamine biosynthesis, thus being useful in a method for restoring toward normal catecholamine balance in a subject in need thereof.

In particular the invention relates to use of said expression construct in a method of treatment of neurological disorders, preferably non-curable degenerative neurological disorders wherein the majority of the patient's experience diminishing treatment response and increased adverse events during prolonged treatment.

The present invention relates primarily to the treatment of Parkinson's disease and L-DOPA Induced Dyskinesia (LID), wherein the present treatment strategy involves the administration of L-DOPA or other dopamine receptor stimulating agents. Current treatment regimens are efficient mainly in the early phase of the disease, but during prolonged treatment most patients develop L-DOPA induced dyskinesia. Development of dyskinesia is believed to be associated with non-continuous delivery of L-DOPA or other dopamine receptor stimulating agents. It is thus a main object of the present invention to refine the present treatment by supplying the compounds necessary for treatment of particularly Parkinson's disease locally where needed and at continuous rates that diminishes any adverse effects.

The present invention relates to expression systems comprising expression systems, to be administered in peripheral tissue for regulating systemic levels of L-DOPA.

In one aspect, the invention relates to an expression system comprising:

a polynucleotide which upon expression encodes a tyrosine hydroxylase (TH; EC 1.14.16.2) polypeptide or a biologically active fragment or variant thereof, wherein said polynucleotide is operably linked to a promoter;
and/or
a polynucleotide which upon expression encodes a GTP-cyclohydrolase 1 (GCH1; EC 3.5.4.16) polypeptide or a biologically active fragment or variant thereof, wherein said polynucleotide is operably linked to a promoter.

In one aspect, the present invention relates to a An expression system comprising:

a first polynucleotide (N1) which upon expression encodes a GTP-cyclohydrolase 1 (GCH1; EC 3.5.4.16) polypeptide or a biologically active fragment or variant thereof, wherein said polynucleotide is operably linked to a first promoter, and wherein the biological activity is enzymatic activity of GCH1;
and
a second polynucleotide (N2) which upon expression encodes a tyrosine hydroxylase (TH; EC 1.14.16.2) polypeptide or a biologically active fragment or variant thereof, wherein said polynucleotide is operably linked to a second promoter, and wherein the biological activity is enzymatic activity of TH;
and
a third polynucleotide (N3) which upon expression encodes a 6-pyruvoyltetrahydropterin synthase (PTPS, EC 4.2.3.12) polypeptide or a biologically active fragment or variant thereof, wherein said polynucleotide is operably linked to a third promoter, and wherein the biological activity is enzymatic activity of PTPS.

In one aspect, the invention concerns an isolated host cell transduced or transfected by the expression system defined herein above.

In another aspect, the invention concerns a pharmaceutical composition comprising the expression system defined herein above, and optionally a pharmaceutically acceptable salt, carrier or adjuvant.

In one aspect, the present invention relates to an expression system as defined herein above for medical use.

In a further aspect, the invention concerns the expression system as defined herein above, for use in a method of treatment of a disease associated with catecholamine dysfunction, wherein said expression system is administered peripherally, i.e. administered outside the CNS.

In another aspect the invention concerns an expression system comprising one or more nucleotide sequences which upon expression encodes one or more polypeptides selected from the group consisting of:

a tyrosine hydroxylase (TH; EC 1.14.16.2) polypeptide or a biologically active fragment or variant thereof; and/or

a GTP-cyclohydrolase 1 (GCH1; EC 3.5.4.16) polypeptide or a biologically active fragment or variant thereof;
for use in a method of treatment of a disease associated with catecholamine dysfunction, wherein said expression system is administered peripherally.

The invention in a further aspect concerns a method for maintaining a therapeutically effective concentration of L-DOPA in blood, said method comprising peripheral administration (i.e. administration outside the CNS) of the expression system defined herein above, to a person in need thereof.

In another aspect the invention concerns a method of treatment and/or prevention of a disease associated with catecholamine dysfunction, said method comprising peripherally administering to a patient in need thereof a therapeutically effective amount of the expression system defined herein above, to a person in need thereof.

In yet another aspect, the invention concerns a method for maintaining a therapeutically effective concentration of L-DOPA in blood of a patient, said method comprising administering to said patient the expression system as defined herein above.

In yet another aspect, the invention concerns a method for reducing, delaying and/or preventing emergence of L-DOPA induced dyskinesia (LID), said method comprising peripherally administering the expression system defined herein above to a patient in need thereof.

In yet another aspect, the invention concerns a method of obtaining and/or maintaining a therapeutically effective concentration of L-DOPA in blood, said method comprising peripherally administering an expression system comprising a nucleotide sequence which upon expression encodes at least one therapeutic polypeptide, wherein the at least one therapeutic polypeptide is a tyrosine hydroxylase (TH; EC 1.14.16.2) polypeptide, or a biologically active fragment or variant thereof.

In one aspect, the invention concerns a kit comprising the pharmaceutical composition defined above, and instructions for use.

### IV. DETAILED DESCRIPTION OF THE INVENTION

### Description of the Drawings

FIG. 1: Overview of L-DOPA biosynthesis

FIG. 2: AAV Vectors for continuous L-DOPA Synthesis in the Liver. A) Bicistronic vector: ITR=inverted terminal repeat sequences, LP1=Liver promoter/enhancer 1, HLP=hybrid liver-specific promoter (see McIntosh J et. al Blood 2013 121(17) 3335-3344), tTH=truncated Tyrosine Hydroxylase (SEQ ID NO: 24), GCH1=GTP cyclohydrolase 1 (SEQ ID NO: 20), WPRE=woodchuck hepatitis virus posttranscriptional regulatory element (SEQ ID NO: 28 or 29). B-E) Monocistronic Vectors. HLP: short liver-specific promoter (McIntosh J et al, Blood. 2013 Apr. 25; 121(17):3335-44) equally strong to LP1.

FIG. 3: Animal Study. A) Mice were randomly allocated to 3 groups of 6 animals. On day one the animals received either no treatment (naïve), or viral vectors as detailed in the table A), respectively. B) Mice were randomly allocated to 2 groups of 2 animals. On day one the animals received viral vectors as detailed in the table B). A) and B): On day 28 the mice received 10 mg/kg beserazide to block decarboxylation of L-DOPA and a COMT inhibitor to block metabolism of L-DOPA by catechol-O-methyl transferase one hour before sacrifice and collection of plasma for L-DOPA assay and liver for immunohistochemistry. The intended dose of COMT inhibitor was tolcapone 30 mg/g administered twice, 4 hours and 1 hour before sacrifice and collection of plasma for L-DOPA assay. C) Illustration of the experimental setup: tail-vein injection followed by low dose of benserazide and entacapone 1 hour before sacrifice and organ harvesting at day 28.

FIG. 4: GCH1 staining. A) Liver sections from naïve mice or mice treated with expression vector scAAV-LP1-GCH1 and/or scAAV-LP1-tTH at a total dose of 7.02×10¹⁰ vg/mouse as described in relation to FIG. 3A. Sections demonstrate transduction of <1%. B) Liver sections from naïve mice or mice treated with expression vectors scAAV-HLP-GCH1 and scAAV-HLP-tTH at a total dose of 3.6×10¹² vg/mouse as described in relation to FIG. 3B. Sections demonstrate transduction of ˜25%.

FIG. 5: Animal Study—Mouse Plasma L-DOPA concentrations. Plasma L-DOPA levels in mice. A) is a table indicating the average L-DOPA level, whereas B) shows a plot indicating the L-DOPA levels for all mice tested. The groups were treated as follows:

A: No vector (control)
B: scAAV-LP1-tTH (3.5×10¹⁰)+scAAV-LP1-GCH1 (3.5×10¹⁰)
C: scAAV-LP1-tTH (7.0×10¹⁰)
D: scAAV HLP-tTH (1.8×10¹²)+scAAV HLP-GCH1 (1.8×10¹²)
E: scAAV-HLP-tTH (3.6×10¹²)

Vectors were administered by an intravenous injection. Plasma was collected 28 days after dosing, one hour after treatment with benserazide (10 mg/kg) and entacapone.

FIG. 6: Animal Study—H&E staining. Liver sections from naïve mice or mice treated with expression vectors scAAV-HLP-GCH1 and/or scAAV-HLP-tTH at a total dose of 3.6×10¹² vg/mouse as described in relation to FIG. 3B were stained with hematoxylin and eosin. The stain shows no signs of tissue damage or leukocyte infiltration.

FIG. 7: Homologous recombination of bicistronic construct. During production of the bicistronic ITR-LP1-GCH1-LP1-tTH-WPRE-ITR vector homologous recombination at the common LP1 sites also results in the production of monocistronic ITR-LP1-tTH-WPRE-ITR.

FIG. 8: A tricistronic expression system. The figure shows an example of an expression system of the invention. The system is tricistronic. The TH gene is under the control of the constitutive promoter EF-1alpha, and comprises an IRES and a sequence encoding 6-pyruvoyltetrahydropterin synthase (PTPS). ITR: inverted terminal repeat sequences. WPRE: Woodchuck hepatitis virus post-transcriptional regulatory element.

### DEFINITIONS

Bicistronic: The term “bicistronic” as used herein may refer to an expression system, a vector or a plasmid. A bicistronic plasmid or vector comprises two genes within a single plasmid or vector. A bicistronic expression system refers to an expression system comprising at least one bicistronic plasmid or at least one bicistronic vector.

Biologically active: The term ‘biologically active’ when used herein in connection with enzymes encoded by the expression system construct of the invention, refers to the enzymatic activity of said enzymes, meaning the capacity to catalyze a certain enzymatic reaction. In particular biologic activity may refer to the enzymatic activity of tyrosine hydroxylase (TH), GTP-cyclohydrolase (GCH-1) or 6-pyruvoyltetrahydropterin synthase (PTPS), or any other enzyme encoded by the expression system of the present disclosure and which may help achieve the therapeutic effect.

Biologically active fragment: The term “biologically active fragment’ as used herein, refers to a part of a polypeptide, including enzymes, sharing the biological activity of the full length polypeptide. The biological activity of the fragment may be smaller than, larger than, or equal to the enzymatic activity of the native full length polypeptide. Biologically active fragments of polypeptides include fragments having at least 70% sequence identity to any one of SEQ ID NO:s 1, 2, 3, 4, 5, 6, 40, 7, 8, 9, 10, 11, 12, 13, 14, 15, 16, 17 or 18. Biologically active fragments of a given polypeptide also include fragments wherein no more than 30% of the amino acid residues of said polypeptide have been deleted, such as no more than 29%, for example no more than 28%, such as no more than 27%, for example no more than 26%, such as no more than 25%, for example no more than 24%, such as no more than 23%, for example no more than 22%, such as no more than 21%, for example no more than 20%, such as no more than 19%, for example no more than 18%, such as no more than 17%, for example no more than 16%, such as no more than 15%, for example no more than 14%, such as no more than 13%, for example no more than 12%, such as no more than 11%, for example no more than 10%, such as no more than 9%, for example no more than 8%, such as no more than 7%, for example no more than 6%, such as no more than 5%, for example no more than 4%, such as no more than 3%, for example no more than 2%, such as no more than 1% of the amino acid residues of said polypeptide have been deleted.

Biologically active variant: The term “biologically active variant’ as used herein, refers to a polypeptide part of a protein, such as an enzyme, having the same biological activity as a native full length protein. The biological activity of the fragment may be smaller than, larger than or equal to the enzymatic activity of the native full length polypeptide.

Catecholamine dysfunction: The term catecholamine dysfunction as used herein refers to abnormalities in catecholamine synthesis, regulation, storage, release, uptake or metabolism as compared to the same parameters in a healthy individual. In particular the catecholamine dysfunction is dopamine dysfunction, such as dopamine deficiency. The person skilled in the art is capable of diagnosing catecholamine dysfunction.

Cognitive impairment: The term ‘cognitive impairment’ used herein refers to a condition with poor mental function, associated with confusion, forgetfulness and difficulty concentrating.

Expression: The term ‘expression’ of a nucleic acid sequence encoding a polypeptide is meant transcription of that nucleic acid sequence as mRNA and/or transcription and translation of that nucleic acid sequence resulting in production of that protein.

Expression cassette: The term ‘expression cassette’ as used herein refers to a genomic sequence that provides all elements required to result in the synthesis of a protein in vivo. This could include, but is not necessarily limited to, a sequence that drives transcription from DNA to mRNA, i.e., a promoter sequence, an open reading frame that includes the genomic sequence for the protein of interest and a 3′ untranslated region that enables polyadenylation of the mRNA.

Expression system: The term ‘expression system’ as used herein refers to a system specifically designed for the production of a gene product, in particular a polypeptide. An expression system comprises a nucleotide sequence which upon expression encodes a polypeptide. Expression systems may be but is not limited to, vectors such as virus vectors, e.g. AAV vector constructs.

Functional in mammalian cells: The term ‘functional in mammalian cells’ as used herein, means a sequence, e.g. a nucleotide sequence such as a expression system, that when introduced into a mammalian cell results in the translation into a biologically active polypeptide.

HLP: The term “hybrid liver-specific promoter” or “HLP” as used herein refers to a promoter as described in McIntosh J et. al Blood 2013 121(17) 3335. The HLP of the present invention comprises a human liver specific enhancer, human liver specific promoter, and a modified intron. In one embodiment the LP1 has the polynucleotide sequence of SEQ ID NO: 45 or a biologically active fragment or variant thereof.

Homology: For the purposes of the present application, the terms sequence ‘homology’ and ‘homologous’ as used herein are to be understood as equivalent to sequence ‘identity’ and ‘identical’.

LP1: The term “liver promoter/enhancer 1” or “LP1” as used herein refers to a promoter as described in Nathwani A C et al. Blood. 2006; 107(7):2653-2661 and Miao H Z et al. Blood. 2004; 103(9):3412-3419. The LP1 of the present inventor comprises a truncated liver-specific enhancer and truncated liver specific promoter. In one embodiment the LP1 has the polynucleotide sequence of SEQ ID NO: 39 or a biologically active fragment or variant thereof.

Operably linked: The term ‘operably linked’ as used herein indicates that the nucleic acid sequence encoding one or more polypeptides of interest and transcriptional regulatory sequences are connected in such a way as to permit expression of the nucleic acid sequence when introduced into a cell.

Peripheral administration: The term ‘peripheral administration’ as used herein refers to peripheral in relation to the central nervous system (CNS). In particular, peripheral administration refers to administration to skeletal muscle and liver tissue. The person of skill in the art is familiar with means for administering a pharmaceutical composition and ingredients thereof to said tissue.

Pharmaceutical composition: or drug, medicament or agent refers to any chemical or biological material, compound, or composition capable of inducing a desired therapeutic effect when properly administered to a patient. Some drugs are sold in an inactive form that is converted in vivo into a metabolite with pharmaceutical activity. For purposes of the present invention, the terms “pharmaceutical composition” and “medicament” encompass both the inactive drug and the active metabolite.

Plasmid: the term ‘plasmid’ refers herein to a polynucleotide which can be naked or packaged within a vector. In the present disclosure, a plasmid is preferably physically separated from the chromosomal DNA of the cell in which it is transferred, and can replicate independently. In some embodiments, the expression system of the present disclosure comprises one or more plasmids, either naked, i.e. unpackaged, or packaged within a vector, as is known in the art.

Polypeptide: The term ‘polypeptide’ as used herein refers to a molecule comprising at least two amino acids. The amino acids may be natural or synthetic. ‘Oligopeptides’ are defined herein as being polypeptides of length not more than 100 amino acids. The term “polypeptide” is also intended to include proteins, i.e. functional biomolecules comprising at least one polypeptide; when comprising at least two polypeptides, these may form complexes, be covalently linked or may be non-covalently linked. The polypeptides in a protein can be glycosylated and/or lipidated and/or comprise prosthetic groups.

Polynucleotide: The term ‘polynucleotide’ used herein refers to a molecule which is an organic polymer molecule composed of nucleotide monomers covalently bonded in a chain. A “polynucleotide” as used herein refers to a molecule comprising at least two nucleic acids. The nucleic acids may be naturally occurring or modified, such as locked nucleic acids (LNA), or peptide nucleic acids (PNA). Polynucleotide as used herein generally pertains to
i) a polynucleotide comprising a predetermined coding sequence, or
ii) a polynucleotide encoding a predetermined amino acid sequence, or
iii) a polynucleotide encoding a fragment of a polypeptide encoded by polynucleotides (i) or (ii), wherein said fragment has at least one predetermined activity as specified herein; and
iv) a polynucleotide the complementary strand of which hybridizes under stringent conditions with a polynucleotide as defined in any one of (i), (ii) and (iii), and encodes a polypeptide, or a fragment thereof, having at least one predetermined activity as specified herein; and
v) a polynucleotide comprising a nucleotide sequence which is degenerate to the nucleotide sequence of polynucleotides (iii) or (iv);
   or the complementary strand of such a polynucleotide.

Promoter: The term ‘promoter’ used herein refers to a region of DNA that facilitates the transcription of a particular gene. A promoter is thus a region of an operon that acts as the initial binding site for RNA polymerase. Promoters are typically located near the genes they regulate, on the same strand and upstream. The term ‘promoter’ as used herein is not limited by structure to classical promoters but should be understood as a region of a nucleotide sequence which has the above described function.

Tricistronic: The term “tricistronic” as used herein may refer to an expression system, a vector or a plasmid. A tricistronic plasmid or vector comprises three genes within a single plasmid or vector. A tricistronic expression system refers to an expression system comprising at least one tricistronic plasmid or at least one tricistronic vector.

Vector: A vector according to the present invention is a DNA molecule used as a vehicle to transfer foreign genetic material into another cell. The four major types of vectors are plasmids, viruses, cosmids, and artificial chromosomes.

Viral vector: A viral vector is to be understood as a virus particle comprising a capsid and a genome. The genome is typically enclosed by the capsid.

### Expression System

Peripheral production and secretion of constant basal L-DOPA into the circulation could achieve similar therapeutic effects as constant infusion into the small intestine via a percutaneous gastrostomy, a mode of therapy currently used to treat PD.

The rationale behind the present invention is to provide a continuous daytime or continuous 24 hours secretion of L-DOPA into the systemic circulation of patients with Parkinson's disease or any other condition in which elevating endogenous peripheral secretion of L-DOPA may be indicated such as hereditary tyrosine hydroxylase deficiency (Wevers et al., 1999) and restless legs syndrome.

The invention is the transduction or transfection of peripheral tissue to produce basal levels of circulating L-dopa sufficient to be therapeutically useful in the treatment of Parkinson's disease or other conditions including tyrosine hydroxylase deficiency or restless leg syndrome.

Transduction of peripheral tissue is achieved by administration of a gene therapy system consisting of an expression system transferring the genetic material enabling targeted peripheral tissue to produce an enzyme able to convert tyrosine to L-3,4-dihydroxyphenylalanine (L-DOPA). The expression system may be provided as one or more vectors as detailed herein below. Preferably, the expression system allows for expression of at least three polypeptides, namely TH, GCH1 and PTPS, and optionally of a fourth polypeptide. In some embodiments, the expression system is provided as two bicistronic vectors or plasmids. In other embodiments, the expression system is provided as one tricistronic vector or plasmid, optionally with a monocistronic vector or plasmid. In other embodiments, the expression system is provided as three or four monocistronic vectors or plasmids.

The cells that are to be targeted by the present expression system may preferably be cells that have a low cell turnover, at least in an adult subject. This is because it is believed, without being bound by theory, that because the vectors or plasmids of the present disclosure do not integrate in the chromosomal DNA of the target cell, the vectors or plasmids are diluted with every cell division. Hence, it is expected that the therapeutic effect fades out with time as cells regenerate. Cells that might be particularly advantageous targets for gene therapy using the present expression system are muscle cells, in particular striated muscle cells, and liver cells.

For example the invention could take the form of gene therapy based on an expression system comprising at least one, such as two, adeno-associated viral vector serotype 8 (targeting hepatic transduction) and delivering the genetic sequence coding for a human Tyrosine Hydroxylase (e.g. hTH2). The transfecting genome could include hepatic specific promoter upstream of a TH gene sequence and may include a woodchuck hepatitis virus post transcriptional regulatory element for maximum expression (WPRE) downstream of the TH gene sequence. Treatment preferably requires supply of tetrahydobiopterin either an oral supplement or produced endogenously by co-transfection of the GPT-cyclohydrolase-1 (GCH1) gene. While co-transfection would remove the need for oral supplementation, reliance on oral supplementation offers the potential to “turn-off” L-dopa production at the site of transfection should this be desired to manage toxicity or to provide periods of reduced L-DOPA production during night. The extent to which GCH1 is required may vary dependent upon the target tissue type (for example liver tissue has higher endogenous levels of GCH1 compared to striated muscle tissue). In preferred embodiments, treatment also requires supply of 6-pyruvoyltetrahydropterin synthase (PTPS, EC 4.2.3.12) which catalyses the conversion of 7,8-dihydroneopterin triphosphate to 6-pyruvoyltetrahydropterin and triphosphate. Preferably, PTPS is produced endogenously by co-transfection of the PTPS gene as described herein.

In another embodiment, the expression system may comprise at least one, such as two adeno-associated viral vector serotype 1 (targeting striated muscle). In such embodiments, any of the promoters linked to the polynucleotides comprised within the expression system may be muscle-specific. The turnover of muscle cells, in particular of mature striated muscle cells, being very low, targeting of muscle cells, such as mature striated muscle cells, is believed to be particularly advantageous.

The expression system may be bicistronic, i.e. comprises at least one bicistronic vector or plasmid. The bicistronic system may further comprise a monocistronic vector or plasmid. Alternatively, the expression system may be tricistronic, i.e. comprises at least one tricistronic vector or plasmid. The tricistronic system may further comprise a monocistronic vector or plasmid.

As with current oral L-DOPA medication a peripheral decarboxylase inhibitor (e.g, benserazine or carbidopa) is preferably administered to block peripheral conversion of the L-DOPA to dopamine thus improving tolerance and bioavailability to the striatum.

In one aspect, the invention relates to an expression system comprising:

a polynucleotide which upon expression encodes a tyrosine hydroxylase (TH; EC 1.14.16.2) polypeptide or a biologically active fragment or variant thereof, wherein said polynucleotide is operably linked to a promoter;
and/or
a polynucleotide which upon expression encodes a GTP-cyclohydrolase 1 (GCH1; EC 3.5.4.16) polypeptide or a biologically active fragment or variant thereof, wherein said polynucleotide is operably linked to a promoter.

In one aspect, the present invention relates to a An expression system comprising:

a first polynucleotide (N1) which upon expression encodes a GTP-cyclohydrolase 1 (GCH1; EC 3.5.4.16) polypeptide or a biologically active fragment or variant thereof, wherein said polynucleotide is operably linked to a first promoter, and wherein the biological activity is enzymatic activity of GCH1;
and
a second polynucleotide (N2) which upon expression encodes a tyrosine hydroxylase (TH; EC 1.14.16.2) polypeptide or a biologically active fragment or variant thereof, wherein said polynucleotide is operably linked to a second promoter, and wherein the biological activity is enzymatic activity of TH;
and
a third polynucleotide (N3) which upon expression encodes a 6-pyruvoyltetrahydropterin synthase (PTPS, EC 4.2.3.12) polypeptide or a biologically active fragment or variant thereof, wherein said polynucleotide is operably linked to a third promoter, and wherein the biological activity is enzymatic activity of PTPS.

In one aspect, the present invention relates to an expression system comprising:

a polynucleotide which upon expression encodes a tyrosine hydroxylase (TH; EC 1.14.16.2) polypeptide or a biologically active fragment or variant thereof, wherein said polynucleotide is operably linked to a promoter;
and/or
a polynucleotide which upon expression encodes a GTP-cyclohydrolase 1 (GCH1; EC 3.5.4.16) polypeptide or a biologically active fragment or variant thereof, wherein said polynucleotide is operably linked to a promoter.

In an embodiment the expression system of the present invention comprises:

a first polynucleotide which upon expression encodes a GTP-cyclohydrolase 1 (GCH1; EC 3.5.4.16) polypeptide or a biologically active fragment or variant thereof, wherein said polynucleotide is operably linked to a first promoter;
and
a second polynucleotide which upon expression encodes a tyrosine hydroxylase (TH; EC 1.14.16.2) polypeptide or a biologically active fragment or variant thereof, wherein said polynucleotide is operably linked to a second promoter.

In an embodiment the expression system of the present invention comprises:

a first polynucleotide which upon expression encodes a GTP-cyclohydrolase 1 (GCH1; EC 3.5.4.16) polypeptide or a biologically active fragment or variant thereof, wherein said polynucleotide is operably linked to a first promoter;
and
a second polynucleotide which upon expression encodes a tyrosine hydroxylase (TH; EC 1.14.16.2) polypeptide or a biologically active fragment or variant thereof, wherein said polynucleotide is operably linked to a second promoter
and
a third polynucleotide which upon expression encodes a 6-pyruvoyltetrahydropterin synthase (PTPS, EC 4.2.3.12) polypeptide or a biologically active fragment or variant thereof, wherein said polynucleotide is operably linked to a third promoter.

In one aspect, the present invention relates to a bicistronic expression system comprising a nucleotide sequence which upon expression encodes:
a tyrosine hydroxylase (TH; EC 1.14.16.2) polypeptide or a biologically active fragment or variant thereof; and
a GTP-cyclohydrolase 1 (GCH1; EC 3.5.4.16) polypeptide or a biologically active fragment or variant thereof.

It will be understood that throughout this disclosure, the terms “first”, “second”, “third” and “fourth” do not refer to a specific order, but instead are used for clarity's sake. Thus, the third polynucleotide of some embodiments may be located between the first and the second polynucleotide.

The bicistronic expression system of the present invention is suitable for administration to an individual such as a human being, for the treatment of diseases and disorders. Thus in one aspect, the present invention relates to an expression system as defined herein above for medical use.

The expression system of the present invention is particularly useful for treating diseases and disorders associated with and/or resulting from, and or/resulting in an imbalance in catecholamine levels. Accordingly, in one aspect, the invention concerns the expression system as defined herein above, for use in a method of treatment of a disease associated with catecholamine dysfunction, wherein said expression system is administered peripherally, i.e. administered outside the CNS.

I.e. the invention in said aspect concerns a bicistronic expression system comprising a nucleotide sequence which upon expression encodes a tyrosine hydroxylase (TH; EC 1.14.16.2) polypeptide or a biologically active fragment or variant thereof; and a GTP-cyclohydrolase 1 (GCH1; EC 3.5.4.16) polypeptide or a biologically active fragment or variant thereof; for use in a method of treatment of a disease associated with catecholamine dysfunction, wherein said expression system is administered peripherally, i.e. administered outside the CNS.

In another aspect the invention concerns an expression system comprising one or more nucleotide sequences which upon expression encodes one or more polypeptides selected from the group consisting of a tyrosine hydroxylase (TH; EC 1.14.16.2) polypeptide or a biologically active fragment or variant thereof; and/or a GTP-cyclohydrolase 1 (GCH1; EC 3.5.4.16) polypeptide or a biologically active fragment or variant thereof; for use in a method of treatment of a disease associated with catecholamine dysfunction, wherein said expression system is administered peripherally.

In one embodiment the expression system for said use comprises a bicistronic expression system as defined herein above.

The expression system may also be a combination of either three monocistronic expression systems or by one monocistronic expression system and one bicistronic expression system. In embodiments where the expression system upon expression encodes four polynucleotides, the system may be a combination of one monocistronic expression system and one tricistronic expression system, or of two monocistronic expression systems and one bicistronic expression system, or of four monocistronic expression systems.

Thus in one embodiment the expression system of the present invention comprises:

a) a bicistronic expression system which upon expression encodes:
i) a tyrosine hydroxylase (TH; EC 1.14.16.2) polypeptide or a biologically active fragment or variant thereof, and
ii) a GTP-cyclohydrolase 1 (GCH1; EC 3.5.4.16) polypeptide or a biologically active fragment or variant thereof.

In another embodiment the expression system of the present invention comprises:

a) a monocistronic expression system which upon expression encodes:
i) a tyrosine hydroxylase (TH; EC 1.14.16.2) polypeptide or a biologically active fragment or variant thereof; and
   b) a monocistronic expression system which upon expression encodes:
i) a GTP-cyclohydrolase 1 (GCH1; EC 3.5.4.16) polypeptide or a biologically active fragment or variant thereof.
In yet another embodiment the expression system of the present invention comprises:
   a) a monocistronic expression system which upon expression encodes:
i) a tyrosine hydroxylase (TH; EC 1.14.16.2) polypeptide or a biologically active fragment or variant thereof; and
   b) a monocistronic expression system which upon expression encodes:
i) GTP-cyclohydrolase 1 (GCH1; EC 3.5.4.16) polypeptide or a biologically active fragment or variant thereof.

In one embodiment the expression system of the present invention comprises:

a) a monocistronic expression system which upon expression encodes:
i) a tyrosine hydroxylase (TH; EC 1.14.16.2) polypeptide or a biologically active fragment or variant thereof; and
   b) a monocistronic expression system which upon expression encodes:
i) GTP-cyclohydrolase 1 (GCH1; EC 3.5.4.16) polypeptide or a biologically active fragment or variant thereof.

Thus in one embodiment the expression system of the present invention comprises:

a) a tricistronic expression system which upon expression encodes:
i) a tyrosine hydroxylase (TH; EC 1.14.16.2) polypeptide or a biologically active fragment or variant thereof, and
ii) a GTP-cyclohydrolase 1 (GCH1; EC 3.5.4.16) polypeptide or a biologically active fragment or variant thereof; and
iii) a 6-pyruvoyltetrahydropterin synthase (PTPS, EC 4.2.3.12) polypeptide or a biologically active fragment or variant thereof.

In another embodiment the expression system comprises:

a) a bicistronic expression system which upon expression encodes:
i) a tyrosine hydroxylase (TH; EC 1.14.16.2) polypeptide or a biologically active fragment or variant thereof, and
ii) a GTP-cyclohydrolase 1 (GCH1; EC 3.5.4.16) polypeptide or a biologically active fragment or variant thereof; and
   b) a monocistronic expression system which upon expression encodes:
iii) a 6-pyruvoyltetrahydropterin synthase (PTPS, EC 4.2.3.12) polypeptide or a biologically active fragment or variant thereof.

In another embodiment the expression system comprises:

a) a bicistronic expression system which upon expression encodes:
i) a tyrosine hydroxylase (TH; EC 1.14.16.2) polypeptide or a biologically active fragment or variant thereof, and
ii) a 6-pyruvoyltetrahydropterin synthase (PTPS, EC 4.2.3.12) polypeptide or a biologically active fragment or variant thereof; and
   b) a monocistronic expression system which upon expression encodes:
iii) a GTP-cyclohydrolase 1 (GCH1; EC 3.5.4.16) polypeptide or a biologically active fragment or variant thereof.

In another embodiment the expression system comprises:

a) a bicistronic expression system which upon expression encodes:
i) a GTP-cyclohydrolase 1 (GCH1; EC 3.5.4.16) polypeptide or a biologically active fragment or variant thereof, and
ii) a 6-pyruvoyltetrahydropterin synthase (PTPS, EC 4.2.3.12) polypeptide or a biologically active fragment or variant thereof; and
   b) a monocistronic expression system which upon expression encodes:
iii) a tyrosine hydroxylase (TH; EC 1.14.16.2) polypeptide or a biologically active fragment or variant thereof.

In another embodiment the expression system of the present invention comprises:

a) a monocistronic expression system which upon expression encodes:
i) a tyrosine hydroxylase (TH; EC 1.14.16.2) polypeptide or a biologically active fragment or variant thereof; and
   b) a monocistronic expression system which upon expression encodes:
ii) a GTP-cyclohydrolase 1 (GCH1; EC 3.5.4.16) polypeptide or a biologically active fragment or variant thereof; and
   c) a monocistronic expression system which upon expression encodes:
iii) a 6-pyruvoyltetrahydropterin synthase (PTPS, EC 4.2.3.12) polypeptide or a biologically active fragment or variant thereof.

The expression system may additionally upon expression encode a fourth polypeptide as detailed herein below.

The purpose of the use of the expression system of the present invention is to obtain and/or maintain a therapeutically effective concentration of L-DOPA in blood of the individual treated with the expression system of the invention.

The enzyme replacement therapy required for in vivo biosynthesis of L-DOPA applied in the present invention relies on one or more of the three enzymes tyrosine hydroxylase (TH; EC 1.14.16.2) and/or GTP-cyclohydrolase 1 (GCH1; EC 3.5.4.16) and/or 6-pyruvoyltetrahydropterin synthase (PTPS, EC 4.2.3.12).

Said enzymes may be expressed as full length polypeptides or as biologically active fragments or variants of the full length enzyme. By biological activity is meant that the capacity to perform at least a fraction of the catalytic activity of the wild type full lengthy enzyme should be retained by the fragment or variant.

Thus in one embodiment the expression system according to the present invention is capable of expressing a GTP-cyclohydrolase 1 (GCH1) polypeptide or a biologically active fragment or variant thereof which is at least 70% identical to a polypeptide selected from the group consisting of SEQ ID NO: 1, SEQ ID NO: 2, SEQ ID NO: 3, SEQ ID NO: 4, SEQ ID NO: 5 and SEQ ID NO: 6.

In one embodiment the expression system according to the present invention is capable of expressing a tyrosine hydroxylase (TH) polypeptide or a biologically active fragment or variant thereof which is at least 70% identical to a polypeptide selected from the group consisting of SEQ ID NO: 40, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, and SEQ ID NO: 17.

In one embodiment the expression system according to the present invention is capable of expressing a 6-pyruvoyltetrahydropterin synthase (PTPS) polypeptide or a biologically active fragment or variant thereof which is at least 70% identical to SEQ ID NO: 41.

The expression system may in principle have any suitable form or structure provided that said form or structure results in a gene product identical or essentially identical or at least having a degree of identity as defined herein, to any one of the enzymes or fragments or variants thereof as defined herein above

### Viral Vectors

Broadly, gene therapy seeks to transfer new genetic material to the cells of a patient with resulting therapeutic benefit to the patient. Such benefits include treatment or prophylaxis of a broad range of diseases, disorders and other conditions.

Ex vivo gene therapy approaches involve modification of isolated cells (including but not limited to stem cells, neural and glial precursor cells, and foetal stem cells), which are then infused, grafted or otherwise transplanted into the patient. See, e.g., U.S. Pat. Nos. 4,868,116, 5,399,346 and 5,460,959. In vivo gene therapy seeks to directly target host patient tissue in vivo.

Viruses useful as gene transfer vectors include papovavirus, adenovirus, vaccinia virus, adeno-associated virus, herpesvirus, and retroviruses. Suitable retroviruses include the group consisting of HIV, SIV, FIV, EIAV, MoMLV. A further group of suitable retroviruses includes the group consisting of HIV, SIV, FIV, EAIV, CIV. Another group of preferred virus vectors includes the group consisting of alphavirus, adenovirus, adeno associated virus, baculovirus, HSV, coronavirus, Bovine papilloma virus, Mo-MLV, preferably adeno associated virus.

Preferred viruses for transduction of hepatic or striated muscle cells are adeno-associated viruses and lentiviruses.

Methods for preparation of AAV are described in the art, e.g. U.S. Pat. No. 5,677,158.

A lentiviral vector is a replication-defective lentivirus particle. Such a lentivirus particle can be produced from a lentiviral vector comprising a 5′ lentiviral LTR, a tRNA binding site, a packaging signal, a promoter operably linked to a polynucleotide signal encoding said fusion protein, an origin of second strand DNA synthesis and a 3′ lentiviral LTR.

### Expression Vectors

Construction of vectors for recombinant expression of the TH and/or GCH1 and/or PTPS polypeptides for use in the invention may be accomplished using conventional techniques which do not require detailed explanation to one of ordinary skill in the art. For review, however, those of ordinary skill may wish to consult Maniatis et al., in Molecular Cloning: A Laboratory Manual, Cold Spring Harbor Laboratory, (NY 1982). Expression vectors may be used for generating producer cells for recombinant production of TH and/or GCH1 and/or PTPS polypeptides for medical use, and for generating therapeutic cells secreting TH and/or GCH1 and/or PTPS polypeptides for naked or encapsulated therapy.

Briefly, construction of recombinant expression vectors employs standard ligation techniques. For analysis to confirm correct sequences in vectors constructed, the genes are sequenced using, for example, the method of Messing, et al., (Nucleic Acids Res., 9: 309-, 1981), the method of Maxam, et al., (Methods in Enzymology, 65: 499, 1980), or other suitable methods which will be known to those skilled in the art.

Size separation of cleaved fragments is performed using conventional gel electrophoresis as described, for example, by Maniatis, et al., (Molecular Cloning, pp. 133-134, 1982).

For generation of efficient expression vectors, these should contain regulatory sequences necessary for expression of the encoded gene in the correct reading frame. Expression of a gene is controlled at the transcription, translation or post-translation levels. Transcription initiation is an early and critical event in gene expression. This depends on the promoter and enhancer sequences and is influenced by specific cellular factors that interact with these sequences. The transcriptional unit of many genes consists of the promoter and in some cases enhancer or regulator elements (Banerji et al., Cell 27: 299 (1981); Corden et al., Science 209: 1406 (1980); and Breathnach and Chambon, Ann. Rev. Biochem. 50: 349 (1981)). Potent promoters and other regulatory elements of the present invention are described in further detail herein below.

In one embodiment the expression system is a vector, such as a viral vector, e.g. a viral vector expression system.

In another embodiment, the expression system is a plasmid vector expression system.

In yet another embodiment the expression system is based on a synthetic vector.

In yet another embodiment the expression system is a cosmid vector or an artificial chromosome.

In certain embodiments, inclusion of an AADC gene into the vector can be disadvantageous for any of a number of reasons. First, it generates a new system that can without modulation convert tyrosine to dopamine. As the transduced cells lack the mechanisms for sequestering the dopamine into vesicles, the dopamine can accumulate rapidly in the cytosol. If the TH enzyme is left with the N-terminal regulatory domain the dopamine produced can directly inhibit the DOPA synthesis through negative feedback which can severely limit the efficacy of the treatment. On the other hand, if the TH enzyme is truncated (e.g. SEQ ID NO: 40), the cytosolic dopamine levels can rapidly increase as the transduced cells also lack mechanisms to release the dopamine.

In one embodiment of the present invention the above defined expression system does not comprise a nucleotide sequence encoding an aromatic amino acid decarboxylase (AADC) polypeptide.

In one embodiment the expression system according to the present invention has a packaging capacity from 1 to 40 kb, for example from 1 to 30 kb, such as from 1 to 20 kb, for example from 1 to 15 kb, such as from 1 to 10, for example from 1 to 8 kb, such as from 2 to 7 kb, for example from 3 to 6 kb, such as from 4 to 5 kb.

In one embodiment the expression system according to the present invention is a viral vector having a packaging capacity from 4.5 to 4.8 kb.

In one embodiment the expression system according to the present invention is a viral vector selected from the group consisting of an adeno associated vector (AAV), adenoviral vector and retroviral vector.

In one embodiment the vector is an integrating vector. In another embodiment the vector is a non-integrating vector.

In one embodiment the present the vector of the present invention is a minimally integrating vector.

In a preferred embodiment the expression system according to the present invention is an adeno associated vector (AAV).

Methods for preparation of AAV vectors are known by those of skill in the art. See e.g. U.S. Pat. No. 5,677,158, U.S. Pat. No. 6,309,634, and U.S. Pat. No. 6,451,306 describing examples of delivery of AAV to the central nervous system.

In one embodiment the AAV vector according to the present invention is selected from the group consisting of serotypes AAV5, AAV1, AAV6, AAV9 and AAV2 vectors. These are preferably used for targeting muscle cells such as myocytes or myoblasts.

In another embodiment the AAV vector according to the present invention is selected from the group consisting of serotypes AAV8, AAV5, AAV2, AAV9 and AAV7 vectors. These are preferably used for targeting cells of the liver, preferably hepatocytes.

Studies have demonstrated (McCarty (2008) Mol Ther. 16(10):1648-56) the efficacy of recombinant adeno-associated virus (rAAV) gene delivery vectors, and recent clinical trials have shown promising results. However, the efficiency of these vectors, in terms of the number of genome-containing particles required for transduction, is hindered by the need to convert the single-stranded DNA (ssDNA) genome into double-stranded DNA (dsDNA) prior to expression. This step can be entirely circumvented through the use of self-complementary vectors, which package an inverted repeat genome that can fold into dsDNA without the requirement for DNA synthesis or base-pairing between multiple vector genomes. The important trade-off for this efficiency is the loss of half the coding capacity of the vector, though small protein-coding genes (up to 55 kd), and any currently available RNA-based therapy, can be accommodated. The increases in efficiency gained with self-complementary AAV (scAAV) vectors have ranged from modest to stunning, depending on the tissue, cell type, and route of administration. Along with the construction and physical properties of self-complementary vectors, the basis of the varying responses in multiple tissues including liver, muscle, and central nervous system (CNS) are outlined in the review by McCarthy.

Accordingly, in one embodiment the AAV vector of the present invention is a self-complementary AAV (scAAV) vector.

In one embodiment the genome of the AAV8 vector is packaged in an AAV capsid other than an AAV8 capsid such as packaged in an AAV5, AAV9, AAV7, AAV6, AAV2 or AAV1 capsid.

In another embodiment the genome of the AAV7 vector is packaged in an AAV capsid other than an AAV7 capsid such as packaged in an AAV8, AAV9. AAV5, AAV6, AAV2 or AAV1 capsid.

In yet another embodiment the genome of the AAV6 vector is packaged in an AAV capsid other than an AAV6 capsid such as packaged in an AAV8, AAV9, AAV7, AAV5, AAV2 or AAV1 capsid.

In yet another embodiment the genome of the AAV5 vector is packaged in an AAV capsid other than an AAV5 capsid such as packaged in an AAV8, AAV9, AAV7, AAV6, AAV2 or AAV1 capsid.

In another embodiment the genome of the AAV2 vector is packaged in an AAV capsid other than an AAV2 capsid such as packaged in an AAV8, AAV9, AAV7, AAV6, AAV5 or AAV1 capsid.

In another embodiment the genome of the genome of the AAV1 vector is packaged in an AAV capsid other than an AAV1 capsid such as packaged in an AAV8, AAV9, AAV7, AAV6, AAV2 or AAV5 capsid.

In another preferred embodiment, the expression system is one or more plasmids, which may be packaged in any of the above-listed vectors, or which may be naked, i.e. unpackaged. In a preferred embodiment, the plasmid is naked.

In one embodiment the vector according to the present invention is capable of infecting or transducing mammalian cells.

In an embodiment the vector according to the present invention is a vector selected from the group comprising SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 52 and SEQ ID NO: 53.

### Promoters

A promoter is a nucleotide sequence that initiates transcription of a particular gene. Promoters are located near the genes which they transcribe, on the same strand and upstream on the nucleotide sequence (towards the 3′ region of the anti-sense strand, also called template strand and non-coding strand). Promoters typically consist of about 100-1000 base pairs.

In an embodiment the expression system of the present invention comprises a first and a second promoter as described herein. In an embodiment said first and said second promoter sequence are different promoter sequences. In another embodiment said first and said second promoter sequence are identical promoter sequences.

In an embodiment the expression system comprises a single promoter located between two of the polynucleotides encoding the three polypeptides TH, GCH1 and PTPS, together with an IRES.

In another embodiment of the expression system of the present invention comprises a polynucleotide which upon expression encodes a tyrosine hydroxylase (TH; EC 1.14.16.2) polypeptide or a biologically active fragment or variant thereof as described herein above, is operably linked to a liver specific promoter.

In another embodiment the expression system according to the present invention comprises a polynucleotide which upon expression encodes a polynucleotide which upon expression encodes a GTP-cyclohydrolase 1 (GCH1; EC 3.5.4.16) polypeptide or a biologically active fragment or variant thereof as described herein above, is operably linked to a liver specific promoter.

In another embodiment the expression system according to the present invention comprises a polynucleotide which upon expression encodes a polynucleotide which upon expression encodes a 6-pyruvoyltetrahydropterin synthase (PTPS, EC 4.2.3.12) polypeptide or a biologically active fragment or variant thereof as described herein above, is operably linked to a liver specific promoter.

In a further embodiment the expression system according to the present invention comprises a promoter as described herein above, wherein the promoter is a liver specific promoter selected from the group consisting of liver promoter/enhancer 1 (LP1) or a biologically active fragment or variant thereof and/or hybrid liver-specific promoter (HLP) or a biologically active fragment or variant thereof.

In another embodiment the expression system according to the present invention comprises a promoter as described herein above, wherein the promoter is a liver specific promoter which is at least 70% identical to a polynucleotide selected from the group consisting of SEQ ID NO: 38 (HLP) and/or SEQ ID NO: 39 (LP1), more preferably at least 75% identical to a polynucleotide selected from the group consisting of SEQ ID NO: 38 and/or SEQ ID NO: 39, more preferably at least 80% identical to a polynucleotide selected from the group consisting of SEQ ID NO: 38 and/or SEQ ID NO: 39, more preferably at least 85% identical to a polynucleotide selected from the group consisting of SEQ ID NO: 38 and/or SEQ ID NO: 39, more preferably at least 90% identical to a polynucleotide selected from the group consisting of SEQ ID NO: 38 and/or SEQ ID NO: 39, more preferably at least 95% identical to a polynucleotide selected from the group consisting of SEQ ID NO: 38 and/or SEQ ID NO: 39, more preferably at least 96% identical to a polynucleotide selected from the group consisting of SEQ ID NO: 38 and/or SEQ ID NO: 39, more preferably at least 97% identical to a polynucleotide selected from the group consisting of SEQ ID NO: 38 and/or SEQ ID NO: 39, more preferably at least 98% identical to a polynucleotide selected from the group consisting of SEQ ID NO: 38 and/or SEQ ID NO: 39, more preferably at least 99% identical to a polynucleotide selected from the group consisting of SEQ ID NO: 38 and/or SEQ ID NO: 39, more preferably 100% identical to a polynucleotide selected from the group consisting of SEQ ID NO: 38 and/or SEQ ID NO: 39.

In another embodiment of the expression system of the present invention comprises a polynucleotide which upon expression encodes a tyrosine hydroxylase (TH; EC 1.14.16.2) polypeptide or a biologically active fragment or variant thereof as described herein above, is operably linked to a muscle specific promoter.

In another embodiment the expression system according to the present invention comprises a polynucleotide which upon expression encodes a polynucleotide which upon expression encodes a GTP-cyclohydrolase 1 (GCH1; EC 3.5.4.16) polypeptide or a biologically active fragment or variant thereof as described herein above, is operably linked to a muscle specific promoter.

In another embodiment the expression system according to the present invention comprises a polynucleotide which upon expression encodes a polynucleotide which upon expression encodes a 6-pyruvoyltetrahydropterin synthase (PTPS, EC 4.2.3.12) polypeptide or a biologically active fragment or variant thereof as described herein above, is operably linked to a muscle specific promoter.

In a further embodiment the expression system according to the present invention comprises a promoter as described herein above, wherein the promoter is a muscle specific promoter selected from the group consisting of pMCK1350, dMCK, tMCK and promoters which are multiple copies of the human slow troponin I gene enhancer, or a biologically active fragment or variant thereof.

In another embodiment the expression system according to the present invention comprises a promoter as described herein above, wherein the promoter is a liver specific promoter which is at least 70% identical to a polynucleotide selected from the group consisting of SEQ ID NO: 38 (HLP) and/or SEQ ID NO: 39 (LP1), more preferably at least 75% identical to a polynucleotide selected from the group consisting of SEQ ID NO: 38 and/or SEQ ID NO: 39, more preferably at least 80% identical to a polynucleotide selected from the group consisting of SEQ ID NO: 38 and/or SEQ ID NO: 39, more preferably at least 85% identical to a polynucleotide selected from the group consisting of SEQ ID NO: 38 and/or SEQ ID NO: 39, more preferably at least 90% identical to a polynucleotide selected from the group consisting of SEQ ID NO: 38 and/or SEQ ID NO: 39, more preferably at least 95% identical to a polynucleotide selected from the group consisting of SEQ ID NO: 38 and/or SEQ ID NO: 39, more preferably at least 96% identical to a polynucleotide selected from the group consisting of SEQ ID NO: 38 and/or SEQ ID NO: 39, more preferably at least 97% identical to a polynucleotide selected from the group consisting of SEQ ID NO: 38 and/or SEQ ID NO: 39, more preferably at least 98% identical to a polynucleotide selected from the group consisting of SEQ ID NO: 38 and/or SEQ ID NO: 39, more preferably at least 99% identical to a polynucleotide selected from the group consisting of SEQ ID NO: 38 and/or SEQ ID NO: 39, more preferably 100% identical to a polynucleotide selected from the group consisting of SEQ ID NO: 38 and/or SEQ ID NO: 39.

In one embodiment the expression system according to the present invention comprises a promoter selective for mammalian cells, such as but not limited to mammalian cells of the liver and skeletal or smooth muscle. In one embodiment the promoter of the invention is specific for a mammalian cell selected from the group consisting of hepatocytes, myocytes and myoblasts.

The promoter may be a naturally occurring promoter or a synthetic promoter.

In one embodiment the expression system according to the present invention comprises a constitutive promoter such as but not limited to one or more promoters selected from the group consisting of p-MCK (promoter for muscle creatine kinase), for example p-MCK1350, promoters which are multiple copies of the human slow troponin I gene enhancer, LB1, HLP, CAG, CBA, CMV, human UbiC, RSV, EF-1alpha, SV40, Mt1, pGK, H1 and/or U3.

In some embodiments, the expression system comprises an EF-1alpha promoter. The EF-1alpha promoter may be located upstream of TH or GCH1.

In one embodiment the expression system according to the present invention comprises an inducible promoter such as but not limited to Tet-On, Tet-Off, Mo-MLV-LTR, Mx1, progesterone, RU486 and/or Rapamycin-inducible promoter.

In one embodiment the expression system according to the present invention comprises a promoter which is specific for liver cells, e.g. hepatocytes. Such promoters includes LP1, hAPO-HCR and/or hAAT. Any liver specific promoter may be useful in the present invention, such as promoters found in genome databases such as the Genbank which can be found at http://www.ncbi.nlm.nih.gov/genbank/, such as the “The Liver Specific Gene Promoter Database” which can be found at http://rulai.cshl.edu/LSPD/.

In another embodiment the expression system according to the present invention comprises one or more promoter(s) specific for muscle cells, such as but not limited to promoters selected from the group consisting of:
a. liver promoter/enhancer 1 (LP1),
b. hybrid liver-specific promoter (HLP) (see McIntosh J et. al Blood 2013 121(17) 3335-3344),
c. muscle specific combined or double promoter using elements of the CMV promoter and SPc5-12,
d. SPc5-12 synthetic muscle specific promotor,
e. muscle specific creatine kinase promoter or abbreviated versions thereof such as dMCK or tMCK, p-MCK1350, or promoters which are multiple copies of the human slow troponin I gene enhancer
f. CMV promoter,
g. muscle CAT promoter,
h. skeletal alpha actin 448 promoter,
i. any active analogues or fragments of any of a through f.

In one embodiment the expression pattern of the promoter can be regulated by a systemically administratable agent. e.g tetracycline on or tetracycline off gene expression systems.

In a preferred embodiment the expression system according to the present invention comprises one or more promoter(s) selected from the group comprising LB1 and HLP. In a more preferred embodiment the expression system according to the present invention comprises one or more promoter(s) selected from the group comprising SEQ ID NO: 38 and SEQ ID NO: 39.

In some embodiments, the expression system comprises a polynucleotide which upon expression encodes TH and a polynucleotide which upon expression encodes GCH1, and further comprises two promoters, where the first promoter is operably linked to TH and the second promoter is operably linked to GCH1.

One or both of the two promoters may be a constitutive promoter selected from the group consisting of LB1, HLP, CAG, CBA, CMV, human UbiC, RSV, EF-1alpha, SV40, Mt1, pGK, H1 and/or U3. In one embodiment, both promoters are EF-1alpha.

One of the two promoters may be a constitutive promoter selected from the group consisting of LB1, HLP, CAG, CBA, CMV, human UbiC, RSV, EF-1alpha, SV40, Mt1, pGK, H1 and/or U3, and the other of the two promoters may be a promoter specific for muscle cells, such as but not limited to promoters selected from the group consisting of:
a. liver promoter/enhancer 1 (LP1),
b. hybrid liver-specific promoter (HLP) (see McIntosh J et. al Blood 2013 121(17) 3335-3344),
c. muscle specific combined or double promoter using elements of the CMV promoter and SPc5-12,
d. SPc5-12 synthetic muscle specific promotor,
e. muscle specific creatine kinase promoter or abbreviated versions thereof such as dMCK or tMCK, p-MCK1350, or promoters which are multiple copies of the human slow troponin I gene enhancer,
f. CMV promoter,
g. muscle CAT promoter,
h. skeletal alpha actin 448 promoter, any active analogues or fragments of any of a through f.

One of the two promoters may be a constitutive promoter selected from the group consisting of LB1, HLP, CAG, CBA, CMV, human UbiC, RSV, EF-1alpha, SV40, Mt1, pGK, H1 and/or U3, and the other of the two promoters may be an inducible promoter such as but not limited to Tet-On, Tet-Off, Mo-MLV-LTR, Mx1, progesterone, RU486 and/or Rapamycin-inducible promoter.

One of the two promoters may be a constitutive promoter selected from the group consisting of LB1, HLP, CAG, CBA, CMV, human UbiC, RSV, EF-1alpha, SV40, Mt1, pGK, H1 and/or U3, and the other of the two promoters may be a promoter which is specific for liver cells, e.g. hepatocytes, as detailed herein above.

### Regulatory Elements

The expression system according to the present invention may in addition to promoters discussed above also comprise other regulatory elements which when included results in modulation of transcription of one or more of the genes encoding TH and/or GCH-1.

In one embodiment the expression system according to the present invention comprises a polyadenylation sequence such as a SV40 polyadenylation sequence. The polyadenylation sequence is typically operably linked to the 3′ end of the nucleic acid sequence encoding said TH and/or GCH-1.

In one embodiment the expression system according to the present invention further comprises a post-transcriptional regulatory element, e.g. a Woodchuck hepatitis virus post-transcriptional regulatory element (WPRE).

In various embodiments said Woodchuck hepatitis virus post-transcriptional regulatory element comprises the sequence of SEQ ID NO: 28 or 29. In a preferred embodiment said Woodchuck hepatitis virus post-transcriptional regulatory element comprises the sequence of SEQ ID NO: 29.

In one embodiment, the expression system further comprises an intron which typically is operably linked to the 5′ end of the TH and/or GCH-1 transcript.

In some embodiments, the expression system comprises an internal ribosome entry site (IRES). Such IRES can allow for translation of a nucleotide sequence to be initiated internally within an mRNA. Thus in some embodiments, the expression system comprises a polynucleotide which upon expression encodes a tyrosine hydroxylase (TH; EC 1.14.16.2) polypeptide or a biologically active fragment or variant thereof, wherein said polynucleotide is operably linked to a promoter;

and
a polynucleotide which upon expression encodes a GTP-cyclohydrolase 1 (GCH1; EC 3.5.4.16) polypeptide or a biologically active fragment or variant thereof, wherein said polynucleotide is operably linked to a promoter,
and
at least one internal ribosome entry site. In such embodiments, the expression system may further comprise a second polynucleotide which upon expression encodes a third polypeptide or a biologically active fragment or variant thereof selected from the group consisting of a tyrosine hydroxylase (TH; EC 1.14.16.2) polypeptide, a GTP-cyclohydrolase 1 (GCH1; EC 3.5.4.16) polypeptide, and a 6-pyruvoyltetrahydropterin synthase (PTPS, EC 4.2.3.12), wherein said second polynucleotide is operably linked to a promoter.

In some embodiments, the polynucleotide encoding GCH1 is located upstream of the polynucleotide encoding TH and the IRES is located downstream of the polynucleotide encoding GCH1 and upstream of the polynucleotide encoding TH. In other embodiments, the polynucleotide encoding TH is located upstream of the polynucleotide encoding GCH1, and the IRES is located downstream of the polynucleotide encoding TH and upstream of the polynucleotide encoding GCH1.

Accordingly, in some embodiments, the expression system allows for independent translation initiation events for TH and for GCH1. The protein synthesis levels of TH and GCH1 may thus be different.

In one embodiment it is of particular interest to regulate the ratio between the enzymes expressed such as the ratio between TH:GCH1.

In one embodiment the TH:GCH1 ratio is 7:1.

In some embodiments, the expression system comprises a polynucleotide which upon expression encodes a GTP-cyclohydrolase 1 (GCH1; EC 3.5.4.16) polypeptide or a biologically active fragment or variant thereof, wherein said polynucleotide is operably linked to a promoter;

and
a polynucleotide which upon expression encodes a 6-pyruvoyltetrahydropterin synthase (PTPS, EC 4.2.3.12) polypeptide or a biologically active fragment or variant thereof, wherein said polynucleotide is operably linked to a promoter,
and
at least one internal ribosome entry site.

In such embodiments, the expression system may further comprise a second polynucleotide which upon expression encodes a tyrosine hydroxylase (TH; EC 1.14.16.2) polypeptide or a biologically active fragment or variant thereof operably linked to a promoter.

In some embodiments, the polynucleotide encoding GCH1 is located upstream of the polynucleotide encoding PTPS and the IRES is located downstream of the polynucleotide encoding GCH1 and upstream of the polynucleotide encoding PTPS. In other embodiments, the polynucleotide encoding PTPS is located upstream of the polynucleotide encoding GCH1, and the IRES is located downstream of the polynucleotide encoding PTPS and upstream of the polynucleotide encoding GCH1.

Accordingly, in some embodiments, the expression system allows for independent translation initiation events for PTPS and for GCH1. The protein synthesis levels of PTPS and GCH1 may thus be different.

In one embodiment it is of particular interest to regulate the ratio between the enzymes expressed such as the ratio between PTPS:GCH1.

In one embodiment the promoter and/or other regulatory element of the expression system of the present invention is capable of directing expression of both PTPS and GCH-1, wherein the ratio of expressed PTPS:GCH1 is at least 3:1, such as at least 4:1, for example at least 5:1, such as at least 6:1, for example at least 7:1, such as at least 10:1, for example 15:1, such as 20:1, for example 25:1, such as 30:1, for example 35:1, such as 40:1, for example 45:1, such as 50:1.

In one embodiment the PTPS:GCH1 ratio is 7:1.

In some embodiments, the expression system comprises a polynucleotide which upon expression encodes a tyrosine hydroxylase (TH; EC 1.14.16.2) polypeptide or a biologically active fragment or variant thereof, wherein said polynucleotide is operably linked to a promoter;

and
a polynucleotide which upon expression encodes a 6-pyruvoyltetrahydropterin synthase (PTPS, EC 4.2.3.12) polypeptide or a biologically active fragment or variant thereof, wherein said polynucleotide is operably linked to a promoter,
and
at least one internal ribosome entry site.

In such embodiments, the expression system may further comprise a second polynucleotide which upon expression encodes GTP-cyclohydrolase 1 (GCH1; EC 3.5.4.16) polypeptide or a biologically active fragment or variant thereof operably linked to a promoter.

In some embodiments, the polynucleotide encoding TH is located upstream of the polynucleotide encoding PTPS and the IRES is located downstream of the polynucleotide encoding TH and upstream of the polynucleotide encoding PTPS. In other embodiments, the polynucleotide encoding PTPS is located upstream of the polynucleotide encoding TH, and the IRES is located downstream of the polynucleotide encoding PTPS and upstream of the polynucleotide encoding TH.

Accordingly, in some embodiments, the expression system allows for independent translation initiation events for PTPS and for TH. The protein synthesis levels of PTPS and TH may thus be different.

In one embodiment it is of particular interest to regulate the ratio between the enzymes expressed such as the ratio between TH:GCH1.

In one embodiment the promoter and/or other regulatory element of the expression system of the present invention is capable of directing expression of both PTPS and TH, wherein the ratio of expressed PTPS:TH is at least 3:1, such as at least 4:1, for example at least 5:1, such as at least 6:1, for example at least 7:1, such as at least 10:1, for example 15:1, such as 20:1, for example 25:1, such as 30:1, for example 35:1, such as 40:1, for example 45:1, such as 50:1.

In one embodiment the PTPS:TH ratio is 7:1.

The ratio between TH:GCH1, PTPS:TH or PTPS:GCH1 can be determined by measuring the activity of the expressed TH and GCH1 enzymes in a sample from a sample host transfected or transduced with the vector as defined herein above.

Alternatively the ratio is determined by measuring the amount of Tetrahydrobiopterin (BH₄) in a sample from a sample host transfected or transduced with the vector as defined herein above.

Alternatively the ratio is determined by the amount of mRNA transcribed in a sample from a sample host transfected or transduced with the vector as defined herein above.

Alternatively the ratio is determined by the amount of protein expressed in a sample from a sample host transfected or transduced with the vector as defined herein above.

### Tyrosine Hydroxylase

Tyrosine hydroxylase, abbreviated TH, is a monooxygenase that catalyzes the conversion of tyrosine to 3,4-dihydroxyphenylalanine (DOPA), a precursor of dopamine. TH activity is modulated by transcriptional and post-translational mechanisms in response to changes in the environment and to neuronal and hormonal stimuli. The most acute regulation of TH activity occurs through post-translational modification of the protein via phosphorylation.

As mentioned, the main function of tyrosine hydroxylase is the conversion of tyrosine to dopamine. TH is primarily found in dopaminergic neurons, but is not restricted to these. The TH gene is essential in embryonic development as the TH knock out genotype is lethal within embryonic day 14 in mice, whereas mice heterozygous for the TH mutation develops normally with only a slight decrease in catecholamine levels. The TH enzyme is highly specific, not accepting indole derivatives, which is unusual as many other enzymes involved in the production of catecholamines do. As the rate-limiting enzyme in the synthesis of catecholamines, TH has a key role in the physiology of adrenergic neurons. Catecholamines, such as dopamine, are major players in the signaling of said adrenergic neurons. Malfunction of adrenergic neurons gives rise to several neurodegenerative disorders in general, such as peripheral neuropathy, amyotrophic lateral sclerosis, Alzheimer's disease, Parkinson's disease, Huntington's disease, ischemic stroke, acute brain injury, acute spinal cord injury, nervous system tumors, multiple sclerosis, peripheral nerve trauma or injury, exposure to neurotoxins, metabolic diseases such as diabetes or renal dysfunctions and damage caused by infectious agents, or to mood disorders such as depression.

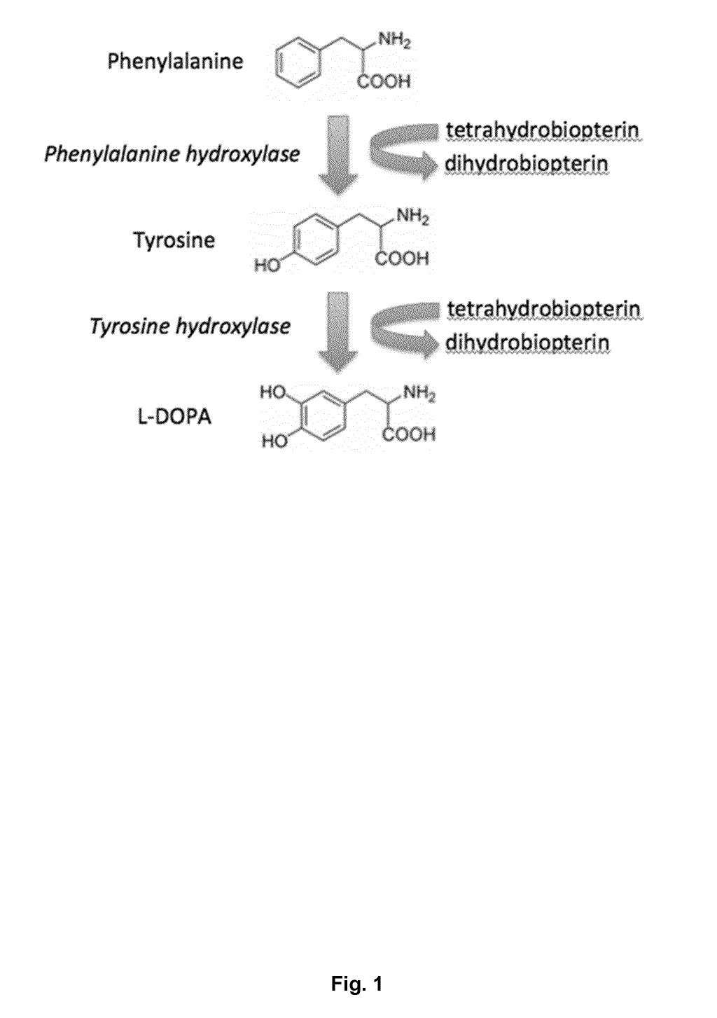

TH administered with the constructs and methods of the present invention may be used in treating Parkinson's disease. As demonstrated in FIG. 1, L-DOPA is biosynthesized from the amino acid L-tyrosine by the enzyme tyrosine hydroxylase (TH).

L-tyrosine is biosynthesized from the amino acid phenylalanine by the enzyme phenylalanine hydrolase (PAH).

Phenylalanine is transported across the plasma membranes of cells including hepatocytes and striated muscle cells (Thöny, 2010).

Tyrosine hydroxylation is the rate-limiting step in the synthesis of catecholamines.

Humans have four isozymes of TH, which differ in their R domains, as pre-mRNA splicing results in additional amino acids following met30.

Intricate regulation of the enzyme is known to occur, which falls into two broad categories: short-term direct regulation of enzyme activity (substrate inhibition by tyrosine (Reed, Lieb, & Nijhout, 2010) feedback inhibition (Kumer & Vrana, 1996), allosteric regulation, and enzyme phosphorylation) and medium-to long-term regulation of gene expression (transcriptional regulation, alternative RNA splicing, RNA stability, translational regulation, and enzyme stability).

Once TH has been synthesized the enzyme is active without phosphorylation, unless it binds with catecholamines in which case it then requires phosphorylation to be activated (Bobrovskaya et al., 2007)

TH is a member of a family of enzymes that also contains the aromatic amino acid hydroxylases (AAAHs) phenylalanine hydroxylase (PheH) and tryptophan hydroxylase (TrpH). All three enzymes perform hydroxylation of the aromatic ring of an amino acid. They all use diatomic oxygen and reduced biopterin in a reaction with a bound iron atom. The iron atom is held in place in the active site cleft by two histidine residues and a glutamate residue, and it must be in the ferrous state to carry out catalysis. In addition to these similarities in the active site, the family shares other features of three-dimensional structure. TH has a multi-domain structure, with an amino-terminal regulatory domain (R) of 160 amino acid residues, followed by a catalytic domain (C) and a much shorter coiled-coil domain at the carboxyl terminus. The enzyme forms a tetramer.

The R domain contains serines at positions 8, 19, 31 and 40. They are all phosphorylated by cAMP-dependent protein kinase (PKA) (Fitzpatrick, 1999). When TH is phosphorylated by PKA, it is less susceptible to feedback inhibition by catecholamines (Daubner, Lauriano, Haycock, & Fitzpatrick, 1992) Although no crystal structures prove it, it is logical to hypothesize that phosphorylation moves the R domain out of the opening of the active site, and dephosphorylation by a phosphatase returns it to its obstructive position (Daubner, Le, & Wang, 2011)

TH is activated after phosphorylation of any of three serine residues in its regulatory domain. Ser40 is phosphorylated mainly by PKA, resulting in a decrease in affinity for catecholamines. Ser31 is phosphorylated by several kinases, resulting in a decrease in K_{M} value for tetrahydrobiopterin. Ser19 is phosphorylated by enzymes that modify only ser19 or both ser19 and -40, and does not result in activation in the absence of other factors. Phosphorylation of ser19 by CaMKII accelerates phosphorylation of ser40 by the same kinase. Any other result of multisite phosphorylation has not yet been established, although stabilization and tighter binding to chaperone proteins are possibilities. Dopamine, norepinephrine, and epinephrine are all feedback inhibitors of TH, and the biggest alteration of TH activity upon ser40 phosphorylation is the change in K_{d} value for catecholamines. DA affinity for TH is 300-fold decreased when the enzyme is phosphorylated (Ramsey & Fitzpatrick, 1998).

Dopamine inhibition of deletion variants of rTyrH lacking the first 32 (THΔ32), the first 68 (THΔ68), the first 76, or the first 120 amino acids has been studied (Daubner & Piper, 1995). The deletion variants were tested for inhibition by preincubation with stoichiometric amounts of dopamine; TyrHD32 was 90% inhibited by dopamine, but TyrHD68 and the other truncates were not inhibited. Furthermore, when dopamine binding and release rates were investigated dopamine was not released from THΔ32 but was rapidly released from THΔ68 (Ramsey & Fitzpatrick, 1998). Dopamine binds 1000-fold more tightly than DOPA, and dihydroxyphenylacetate binds 100-fold times less tightly than DOPA (Ramsey & Fitzpatrick, 2000).

TH also contains a second low affinity (K(D)=90 nM) dopamine-binding site, which is present in both the non-phosphorylated and the Ser40-phosphorylated forms of the enzyme. Binding of dopamine to the high-affinity site decreases V(max) and increases the K_{M} for the cofactor tetrahydrobiopterin, while binding of dopamine to the low-affinity site regulates TH activity by increasing the K_{M} for tetrahydrobiopterin. Kinetic analysis indicates that both sites are present in each of the four human TH isoforms. Dissociation of dopamine from the low-affinity site increases TH activity 12-fold for the non-phosphorylated enzyme and 9-fold for the Ser40-phosphorylated enzyme. The low-affinity dopamine-binding site has the potential to be the primary mechanism responsible for the regulation of catecholamine synthesis under most conditions (Gordon, Quinsey, Dunkley, & Dickson, 2008).

Truncated TH lacking approximately the first 160 amino acids of the N terminus regulatory domain is still active in catalyzing the conversion of tyrosine to DOPA (e.g. SEQ ID NO: 40). Another truncated version of TH is to remove the first 155 amino acids. The serines at position 8, 19, 31, 40 are considered particularly important site for phosphorylation/dephosphorylaion in the regulation of feedback control or TH. Thus other truncations may as well be useful in the present invention. In an embodiment TH of the present invention is lacking the first 10-300 amino acids, such as lacking the first 100-250 amino acids, such as lacking the first 130-210 amino acids, preferably such as lacking the first 140-170 amino acids, more preferably such as lacking the first 150-160 amino acids.

Given that the three aromatic amino acid hydroxylases TH, phenylalanine hydroxylase (PAH) and tryptophan hydroxylase (TRPH) all share a highly homologous catalytic domain of approximately 330 amino acids at the C terminus it has been proposed that substrate specificity is in part due to the regulatory domain of each. Chimeric mutants of TH and PAH in which the R domain of each enzyme is attached to the C domain of the other were constructed (Daubner, Hillas, & Fitzpatrick, 1997). Using these chimeric mutants, as well as truncated mutants lacking their N-terminal R domains, and the wild-type enzymes, Daubner et al demonstrated the roles of the amino-terminal domains in defining the amino acid substrate specificity of these enzymes. The truncated proteins showed low binding specificity for either amino acid. Attachment of either regulatory domain greatly increased the specificity, but the specificity was determined by the catalytic domain in the chimeric proteins.

The polynucleotide sequences encoding TH in the present invention is set forth in SEQ ID NO: 23, SEQ ID NO: 24, SEQ ID NO: 25, SEQ ID NO: 26 and SEQ ID NO: 27. In a preferred embodiment, the present invention relates to the polynucleotide encoding the TH polypeptide comprising a sequence identity of at least 70% to SEQ ID NO: 23, SEQ ID NO: 24, SEQ ID NO: 25, SEQ ID NO: 26 and SEQ ID NO: 27 more preferably 75% sequence identity, for example at least 80% sequence identity, such as at least 85% sequence identity, for example at least 90% sequence identity, such as at least 95% sequence identity, for example at least 96% sequence identity, such as at least 97% sequence identity, for example at least 98% sequence identity, such as at least 99% sequence identity with the SEQ ID NO: 23, SEQ ID NO: 24, SEQ ID NO: 25, SEQ ID NO: 26 and SEQ ID NO: 27.

The polynucleotide, encoding TH, comprised in the expression system construct of the present invention may also encode biologically active fragments or variants of the TH polypeptide.

In a preferred embodiment, such fragments or variants of the TH polynucleotide encode a TH polypeptide which comprises at least 50 contiguous amino acids, such as 75 contiguous amino acids, for example 100 contiguous amino acids, such as 150 contiguous amino acids, for example 200 contiguous amino acids, such as 250 contiguous amino acids, for example 300 contiguous amino acids, such as 350 contiguous amino acids, for example 400 contiguous amino acids, such as 450 contiguous amino acids.

In one embodiment the biologically active fragment is the catalytic domain of tyrosine hydroxylase (SEQ ID NO: 13) or (SEQ ID NO: 40).

In certain embodiments, the specified tyrosine hydroxylase is a mutated and/or substituted variant of SEQ ID NO: 40, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16 and SEQ ID NO: 17 of the encoded TH polypeptide of the present invention are also covered. In one embodiment, the substitutions in the amino acid sequence are conservative, wherein the amino acid is substituted with another amino acid with similar chemical and/or physical characteristics. Mutations may occur in one or more sites within SEQ ID NO: 40, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16 and SEQ ID NO: 17 and or in the encoded TH polypeptide. In a preferred embodiment, the present invention relates to any mutation that renders TH biologically active, such as for example neutral mutations or silent mutations. In a more preferred embodiment, the present invention relates to mutations, wherein one or more of the serine residues S8, S19, S31, S40 or S404 of any one of SEQ ID NO: 7 or equivalent amino acid residue in any one of, SEQ ID NO: 40, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16 and SEQ ID NO: 17 have been altered.

In one embodiment the biologically active variant is a mutated tyrosine hydroxylase polypeptide, wherein one or more of the residues S19, S31, S40 or S404 of SEQ Id NO: 7 have been altered to another amino acid residue.

In one embodiment, the tyrosine hydroxylase (TH) polypeptide expressed by the expression system construct according to the present invention is at least 70% identical to a polypeptide selected from the group consisting of SEQ ID NO: 40, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16 and SEQ ID NO: 17, more preferably at least 75% identical to a polypeptide selected from the group consisting of SEQ ID NO: 40, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16 and SEQ ID NO: 17, more preferably at least 80% identical to a polypeptide selected from the group consisting of SEQ ID NO: 40, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16 and SEQ ID NO: 17, more preferably at least 85% identical to a polypeptide selected from the group consisting of SEQ ID NO: 40, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16 and SEQ ID NO: 17, more preferably at least 90% identical to a polypeptide selected from the group consisting of SEQ ID NO: 40, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16 and SEQ ID NO: 17, more preferably at least 95% identical to a polypeptide selected from the group consisting of SEQ ID NO: 40, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16 and SEQ ID NO: 17, more preferably at least 96% identical to a polypeptide selected from the group consisting of SEQ ID NO: 40, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16 and SEQ ID NO: 17, more preferably at least 97% identical to a polypeptide selected from the group consisting of SEQ ID NO: 40, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16 and SEQ ID NO: 17, more preferably at least 98% identical to a polypeptide selected from the group consisting of SEQ ID NO: 40, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16 and SEQ ID NO: 17, more preferably at least 99% identical to a polypeptide selected from the group consisting of SEQ ID NO: 40, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16 and SEQ ID NO: 17, more preferably 100% identical to a polypeptide selected from the group consisting of SEQ ID NO: 40, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16 and SEQ ID NO: 17.

### GTP-Cyclohydrolase 1

GTP-cyclohydrolase I (GCH1) is a member of the GTP cyclohydrolase family of enzymes. GCH1 is part of the folate and biopterin biosynthesis pathways. GCH1 is the first and rate-limiting enzyme in tetrahydrobiopterin (BH₄) biosynthesis, catalyzing the conversion of GTP into 7,8-DHNP-3′-TP. BH₄ is an essential cofactor required by the aromatic amino acid hydroxylase (AAAH) in the biosynthesis of the monoamine neurotransmitters serotonin (5-hydroxytryptamine (5-HT), melatonin, dopamine, noradrenaline, and adrenaline. Mutations in this gene are associated with malignant phenylketonuria and hyperphenylalaninemia, as well as L-DOPA-responsive dystonia.

Several alternatively spliced transcript variants encoding different isoforms have been described; however, not all of the variants give rise to a functional enzyme.

GCH1 has a number of clinical implications, involving several disorders. Defects in GCH1 are the cause of GTP cyclohydrolase 1 deficiency (GCH1D; also known as atypical severe phenylketonuria due to GTP cyclohydrolase I deficiency. GCH1D is one of the causes of malignant hyperphenylalaninemia due to tetrahydrobiopterin deficiency. It is also responsible for defective neurotransmission due to depletion of the neurotransmitters dopamine and serotonin, resulting in diseases such as Parkinson's disease. The principal symptoms include: psychomotor retardation, tonicity disorders, convulsions, drowsiness, irritability, abnormal movements, hyperthermia, hypersalivation, and difficulty swallowing. Some patients may present a phenotype of intermediate severity between severe hyperphenylalaninemia and mild dystonia type 5 (dystonia-parkinsonism with diurnal fluctuation). In this intermediate phenotype, there is marked motor delay, but no mental retardation and only minimal, if any, hyperphenylalaninemia. Defects in GCH1 are the cause of dystonia type 5 (DYT5); also known as progressive dystonia with diurnal fluctuation, autosomal dominant Segawa syndrome or dystonia-parkinsonism with diurnal fluctuation. DYT5 is a DOPA-responsive dystonia. Dystonia is defined by the presence of sustained involuntary muscle contractions, often leading to abnormal postures. DYT5 typically presents in childhood with walking problems due to dystonia of the lower limbs and worsening of the dystonia towards the evening. It is characterized by postural and motor disturbances showing marked diurnal fluctuation. Torsion of the trunk is unusual. Symptoms are alleviated after sleep and aggravated by fatigue and exercise. There is a favorable response to L-DOPA without side effects.

GCH1 administered with the constructs and methods of the present invention may be used in treating Parkinson's disease.

The polynucleotide sequence encoding GCH1 in the present invention is set forth in SEQ ID NO: 30. In a preferred embodiment, the present invention relates to SEQ ID NO: 30 and sequence variants of the polynucleotide encoding the GCH1 polypeptide comprising a sequence identity of at least 70% to SEQ ID NO: 30, more preferably 75% sequence identity, for example at least 80% sequence identity, such as at least 85% sequence identity, for example at least 90% sequence identity, such as at least 95% sequence identity, for example at least 96% sequence identity, such as at least 97% sequence identity, for example at least 98% sequence identity, such as at least 99% sequence identity with the SEQ ID NO: 30.

The polynucleotide, encoding GCH1, comprised in the expression system construct of the present invention may also encode biologically active fragments or variants of the GCH1 polypeptide.

In a preferred embodiment, such fragments or variants of the GCH1 polynucleotide encoded by the present invention comprise at least 50 contiguous amino acids, such as 75 contiguous amino acids, for example 100 contiguous amino acids, such as 150 contiguous amino acids, for example 200 contiguous amino acids, such as 250 contiguous amino acids, wherein any amino acid specified in the sequence in question is altered to a different amino acid, provided that no more than 15 of the amino acids in said fragment or variant are so altered.

Mutated and substituted versions of SEQ ID NO: 1, SEQ ID NO: 2, SEQ ID NO: 3, SEQ ID NO: 4, SEQ ID NO: 5 and SEQ ID NO: 6 and the encoded GCH1 polypeptide of the present invention are also covered. In one embodiment, the substitutions in the amino acid sequence are conservative, wherein the amino acid is substituted with another amino acid with similar chemical and/or physical characteristics. Mutations may occur in one or more sites within SEQ ID NO: 1, SEQ ID NO: 2, SEQ ID NO: 3, SEQ ID NO: 4, SEQ ID NO: 5 and SEQ ID NO: 6 and or in the encoded GCH1 polypeptide. In a preferred embodiment, the present invention relates to any mutation that renders GCH1 biologically active, such as for example neutral mutations or silent mutations.

In one embodiment, the biologically active fragment expressed by the expression system construct according to the present invention comprises at least 50 contiguous amino acids, wherein any amino acid specified in the selected sequence is altered to a different amino acid, provided that no more than 15 of the amino acid residues in the sequence are so altered.

In one embodiment, the GTP-cyclohydrolase 1 (GCH1) polypeptide expressed by the expression system construct according to the present invention is at least 70% identical to a polypeptide selected from the group consisting of SEQ ID NO: 1, SEQ ID NO: 2, SEQ ID NO: 3, SEQ ID NO: 4, SEQ ID NO: 5 and SEQ ID NO: 6, more preferably at least 75% identical to a polypeptide selected from the group consisting of SEQ ID NO: 1, SEQ ID NO: 2, SEQ ID NO: 3, SEQ ID NO: 4, SEQ ID NO: 5 and SEQ ID NO: 6, more preferably at least 80% identical to a polypeptide selected from the group consisting of SEQ ID NO: 1, SEQ ID NO: 2, SEQ ID NO: 3, SEQ ID NO: 4, SEQ ID NO: 5 and SEQ ID NO: 6, more preferably at least 85% identical to a polypeptide selected from the group consisting of SEQ ID NO: 1, SEQ ID NO: 2, SEQ ID NO: 3, SEQ ID NO: 4, SEQ ID NO: 5 and SEQ ID NO: 6, more preferably at least 90% identical to a polypeptide selected from the group consisting of SEQ ID NO: 1, SEQ ID NO: 2, SEQ ID NO: 3, SEQ ID NO: 4, SEQ ID NO: 5 and SEQ ID NO: 6, more preferably at least 95% identical to a polypeptide selected from the group consisting of SEQ ID NO: 1, SEQ ID NO: 2, SEQ ID NO: 3, SEQ ID NO: 4, SEQ ID NO: 5 and SEQ ID NO: 6, more preferably at least 96% identical to a polypeptide selected from the group consisting of SEQ ID NO: 1, SEQ ID NO: 2, SEQ ID NO: 3, SEQ ID NO: 4, SEQ ID NO: 5 and SEQ ID NO: 6, more preferably at least 97% identical to a polypeptide selected from the group consisting of SEQ ID NO: 1, SEQ ID NO: 2, SEQ ID NO: 3, SEQ ID NO: 4, SEQ ID NO: 5 and SEQ ID NO: 6, more preferably at least 98% identical to a polypeptide selected from the group consisting of SEQ ID NO: 1, SEQ ID NO: 2, SEQ ID NO: 3, SEQ ID NO: 4, SEQ ID NO: 5 and SEQ ID NO: 6, more preferably at least 99% identical to a polypeptide selected from the group consisting of SEQ ID NO: 1, SEQ ID NO: 2, SEQ ID NO: 3, SEQ ID NO: 4, SEQ ID NO: 5 and SEQ ID NO: 6, more preferably 100% identical to a polypeptide selected from the group consisting of SEQ ID NO: 1, SEQ ID NO: 2, SEQ ID NO: 3, SEQ ID NO: 4, SEQ ID NO: 5 and SEQ ID NO: 6.

6-pyruvoyltetrahydropterin synthase (PTPS, EC 4.2.3.12)

6-pyruvoyltetrahydropterin synthase (PTPS, EC 4.2.3.12) is an enzyme which catalyses the conversion of 7,8-dihydroneopterin triphosphate to 6-pyruvoyltetrahydropterin and triphosphate. The reaction is reversible. 6-pyruvoyltetrahydropterin is an intermediate in the biosynthesis of tetrahydrobiopterin (BH₄). In particular, PTPS appears to facilitate production and activity of GCH1. BH₄ has been reported to play a role in the stability and activity of phenylalanine hydroxylase, and thereby in the biosynthesis of L-DOPA. PTPS is expressed in the liver. Without wishing to be bound by theory, it is hypothesised that the naïve, endogenous expression levels of PTPS in the liver are sufficient to permit biosynthesis of L-DOPA. Accordingly, the present expression systems to be transfected in a host cell as detailed below may further comprise a polynucleotide which upon expression encodes a 6-pyruvoyltetrahydropterin synthase (PTPS, EC 4.2.3.12). This is of particular relevance in embodiments where the host cell is not a liver cell, for example the host cell is a muscle cell such as a myocyte or a muscle cell precursor such as a myoblast.

PTPS administered with the constructs and methods of the present invention may be used in treating Parkinson's disease.

The polynucleotide sequence encoding PTPS in the present invention is set forth in SEQ ID NO: 41. In a preferred embodiment, the present invention relates to SEQ ID NO: 41 and sequence variants of the polynucleotide encoding the PTPS polypeptide comprising a sequence identity of at least 70% to SEQ ID NO: 41, more preferably 75% sequence identity, for example at least 80% sequence identity, such as at least 85% sequence identity, for example at least 90% sequence identity, such as at least 95% sequence identity, for example at least 96% sequence identity, such as at least 97% sequence identity, for example at least 98% sequence identity, such as at least 99% sequence identity with the SEQ ID NO: 41.

The polynucleotide, encoding PTPS, comprised in the expression system construct of the present invention may also encode biologically active fragments or variants of the PTPS polypeptide.

In a preferred embodiment, such fragments or variants of the PTPS polynucleotide encoded by the present invention comprise at least 50 contiguous amino acids, such as 75 contiguous amino acids, for example 100 contiguous amino acids, such as 150 contiguous amino acids, for example 200 contiguous amino acids, such as 250 contiguous amino acids, wherein any amino acid specified in the sequence in question is altered to a different amino acid, provided that no more than 15 of the amino acids in said fragment or variant are so altered.

Mutated and substituted versions of SEQ ID NO: 41 and the encoded PTPS polypeptide of the present invention are also covered. In one embodiment, the substitutions in the amino acid sequence are conservative, wherein the amino acid is substituted with another amino acid with similar chemical and/or physical characteristics. Mutations may occur in one or more sites within SEQ ID NO: 41 and or in the encoded PTPS polypeptide. In a preferred embodiment, the present invention relates to any mutation that renders PTPS biologically active, such as for example neutral mutations or silent mutations.

In one embodiment, the biologically active fragment expressed by the expression system construct according to the present invention comprises at least 50 contiguous amino acids, wherein any amino acid specified in the selected sequence is altered to a different amino acid, provided that no more than 15 of the amino acid residues in the sequence are so altered.

In one embodiment, the PTPS polypeptide expressed by the expression system construct according to the present invention is at least 70% identical to SEQ ID NO: 41, more preferably at least 75% identical to SEQ ID NO; 41, more preferably at least 80% identical to SEQ ID NO: 41, more preferably at least 85% identical to SEQ ID NO: 41, more preferably at least 90% identical to SEQ ID NO: 41, more preferably at least 95% identical to SEQ ID NO: 41, more preferably at least 96% identical to SEQ ID NO: 41, more preferably at least 97% identical to SEQ ID NO: 41, more preferably at least 98% identical to SEQ ID NO: 41, more preferably at least 99% identical to SEQ ID NO: 41, more preferably 100% identical to SEQ ID NO: 41.

### Cell Lines

In one aspect the invention relates to isolated host cells genetically modified with the vector/expression system according to the invention.

The invention also relates to cells suitable for biodelivery of TH and/or GCH-1 via naked cells, which are genetically modified to overexpress TH and/or GCH-1, and which can be transplanted to the patient to deliver bioactive TH and/or GCH-1 polypeptide locally in the peripheral tissue of interest. Such cells may broadly be referred to as therapeutic cells.

For ex vivo gene therapy, the preferred group of cells includes isolated host cell transduced or transfected by the expression system as defined herein above. The host cell is selected from the group consisting of eukaryotic cells, preferably mammalian cells, more preferably primate cells, more preferably human cells.

In one embodiment the host cells are transfected ex-vivo and subsequently administered such as transplanted into a mammal.

In one embodiment the host cell is selected from the group consisting of hepatocytes, myocytes and myoblasts.

In one embodiment said mammalian cell is a liver cell such as a hepatocyte.

In another embodiment the mammalian cell is a muscle cell such as a myocyte or a muscle cell precursor such as a myoblast. In such embodiments, the expression system preferably also includes a polynucleotide encoding 6-pyruvoyltetrahydropterin synthase (PTPS) operatively linked to a promoter.

### Medical Use of the Expression System

As indicated herein above the expression system according to the present invention is intended for medical use.

In a highly preferred aspect, the expression system according to the present invention is for use in peripheral administration for the treatment of a disease or disorder associated with catecholamine dysfunction.

Accordingly, in one embodiment, the expression system according to the present invention is particularly well suited for use in a method of maintaining a therapeutically effective concentration of L-DOPA in blood, said method comprising peripheral administration of said expression system to a person in need thereof.

A therapeutically effective amount or in other words the therapeutic range for plasma L-DOPA is normally within the range of 0.2-1.5 mg/L, but the correlation between plasma level at any point in time and therapeutic status varies over the course of the day. This variation is related to factors such as the lag between reaching plasma and crossing the blood brain barrier and competition with other amino acids for active transport across the blood brain barrier.

Systemic gene therapy induced basal levels of L-DOPA smoothen out, which prevents troughs in circulating levels of L-DOPA, which troughs would otherwise occur if traditional oral L-DOPA was given. Accordingly the present invention is useful for treating and/or preventing L-DOPA induced dyskinesia (LID).

The expression system is thus designed and formulated for peripheral administration with the aim of treating of a condition or disease associated with catecholamine dysfunction such as Parkinson's Disease and L-DOPA induced dyskinesia.

The invention in a further aspect concerns a method for maintaining a therapeutically effective concentration of L-DOPA in blood, said method comprising peripheral administration (i.e. administration outside the CNS) of the expression system defined herein above, to a person in need thereof.

In another aspect the invention concerns a method of treatment and/or prevention of a disease associated with catecholamine dysfunction, said method comprising peripherally administering to a patient in need thereof a therapeutically effective amount of the expression system defined herein above, to a person in need thereof.

In yet another aspect, the invention concerns a method for maintaining a therapeutically effective concentration of L-DOPA in blood of a patient, said method comprising administering to said patient the expression system as defined herein above.

In yet another aspect, the invention concerns a method for reducing, delaying and/or preventing emergence of L-DOPA induced dyskinesia (LID), said method comprising peripherally administering the expression system defined herein above to a patient in need thereof.

In yet another aspect, the invention concerns a method of obtaining and/or maintaining a therapeutically effective concentration of L-DOPA in blood, said method comprising peripherally administering a vector comprising a nucleotide sequence which upon expression encodes at least one therapeutic polypeptide, wherein the at least one therapeutic polypeptide is a tyrosine hydroxylase (TH; EC 1.14.16.2) polypeptide, or a biologically active fragment or variant thereof.

Indications treatable by the present invention include indications associated with catecholamine dysfunction, in particular catecholamine deficiency such as dopamine deficiency.

In one embodiment the disease associated with catecholamine dysfunction is a disease, disorder or damage of the central and/or peripheral nervous system such as a neurodegenerative disorder.

In one embodiment the disease treatable by the present invention is a disease of the basal ganglia.

In one embodiment the expression system according to the present invention is administered peripherally for use in the treatment of a disease selected from the group consisting of Parkinson's Disease (PD), dyskinesia, DOPA responsive dystonia, ADHD, schizophrenia, depression, vascular parkinsonism, essential tremor, chronic stress, genetic dopamine receptor abnormalities, chronic opoid, cocaine, alcohol or marijuana use, adrenal insufficiency, hypertension, hypotension, noradrenaline deficiency, post-traumatic stress disorder, pathological gambling disorder, dementia, Lewy body dementia and hereditary tyrosine hydroxylase deficiency.

In an embodiment the expression system and/or the host cell according to the present invention is for use in a method of treatment of Parkinson's disease, atypical Parkinson's disease including conditions such as Multiple System Atrophy, Progressive Supranuclear Palsy, Vascular or arteriosclerotic Parkinson's disease, Drug induced Parkisonism and GTP cyclohydrolase 1 deficiency and/or any dystonic conditions due to dopamine deficiency.

In particular the expression system is useful for the treatment of Parkinson's Disease (PD) and symptoms and conditions associated therewith

In one aspect the present invention concerns a method for maintaining a therapeutically effective concentration of L-DOPA in blood of a patient, said method comprising administering to said patient the expression system as defined herein above.

In one aspect, the present invention concerns a method for reducing, delaying and/or preventing emergence of L-DOPA induced dyskinesia (LID), said method comprising peripherally administering the expression system as defined herein to a patient in need thereof.

### Administration of the Expression System

In order to achieve appropriate effect of the present invention it is necessary to administer the expression system peripherally, i.e. locally or systemically but in either case outside the CNS—although some of the expression system may eventually penetrate the CNS.

The expression system of the present invention is generally administered in the form of a suitable pharmaceutical composition. Accordingly, the present invention also relates to a pharmaceutical composition comprising the expression system as defined herein. Such compositions typically contain the expression system and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the expression system, use thereof in the compositions is contemplated. Supplementary active compounds can also be incorporated into the compositions.

A pharmaceutical composition of the invention is formulated to be compatible with its intended route of administration. Examples of suitable routes of administration include parenteral, e.g., intramuscular, intravenous, intrahepatic, intradermal, subcutaneous and transmucosal administration, or isolated limb perfusion.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition.

Sterile injectable solutions can be prepared by incorporating the expression system in the required amount in an appropriate solvent with one or a combination of ingredients enumerated above, as required, followed by filtered sterilization.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one embodiment, the agent is prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Thus in one aspect the invention concerns a pharmaceutical composition comprising the expression system as defined herein above.

The pharmaceutical compositions can be included in a container, pack, or dispenser together with instructions for administration.

Thus in one aspect, the invention concerns a kit comprising the pharmaceutical composition defined above, and instructions for use.

As described herein above, it is an aim of the present invention to provide an expression system for gene therapy which expression system is administered peripherally in relation to the CNS, i.e. outside the CNS in order to avoid use of brain surgery, including injection into the brain.

In one embodiment the expression system according to the present invention is administered peripherally by intravenous administration.

In one embodiment the administration is in the portal vein. Such administration targets the liver.

The expression system according to the present invention may also be administered peripherally by intrahepatic administration.

In one embodiment the expression system according to the present invention is administered peripherally by intramuscular administration.

In one embodiment the expression system according to the present invention is administered by isolated limb perfusion. In this case, naked plasmid DNA can be administered as described in Hagstrom et al. (2004) Mol. Ther. 10(2): 386-398.

Multiple administrations may be needed for the expression system to have a therapeutic effect. In some embodiments, the expression system is administered at least once, such as once, twice, thrice, four times, five times, six times, seven times, eight times, nine times, ten times, or more.

The dosage to be administered may depend on multiple factors including the individual to be treated, the expression system and the promoter. In some embodiments of the invention, the expression system may be administered in a dosage of at least 1×10¹¹ vg/kg body weight, such as at least 1×10¹² vg/kg body weight. In some embodiments of the invention, the expression system may be administered in a dosage of at least 1×10¹¹ vg/kg muscle, such as at least 1×10¹² vg/kg muscle. Such dosages may for example be applicable for a human being.

### Combination Treatment

The treatment regimen by the expression system defined herein above may be supplemented by other suitable compounds. In one such embodiment the invention further comprises supplementing the administration of the expression system with systemic administration of a therapeutically effective amount of L-DOPA.

In one embodiment a therapeutically effective amount of tetrahydrobiopterin (BH₄) or an analogue thereof is administered to the patient receiving gene therapy through the expression system of the present invention.

In one embodiment the BH₄ analogue is sapropterin.

In one embodiment of the invention a therapeutically effective amount of a peripheral decarboxylase inhibitor is administered. The decarboxylase inhibitor is typically selected from the group consisting of benserazine and carbidopa.

In a further embodiment a therapeutically effective amount of a catechol-O-methyltransferase (COMT) inhibitor is administered to the patient in need thereof.

The catechol-O-methyltransferase (COMT) inhibitor is typically selected from the group consisting of tolcapone, entacapone and nitecapone.

In certain embodiments, BH₄, decarboxylase inhibitor and/or catechol-O-methyltransferase (COMT) inhibitor is/are administered orally.

Alternatively, the BH₄, decarboxylase inhibitor and/or catechol-O-methyltransferase (COMT) inhibitor is/are administered intravenously or intramuscularly.

In one combination treatment, the administration of BH₄, decarboxylase inhibitors and/or COMT-inhibitors and/or analogues thereof, is by systemic administration.

In one combination treatment, the administration of BH₄, decarboxylase inhibitors and/or COMT-inhibitors and analogues thereof, is by enteral or parenteral administration.

In one combination treatment, the administration of BH₄, decarboxylase inhibitors and/or COMT-inhibitors and analogues thereof, is by oral, intravenous or intramuscular administration.

### VII. EXAMPLES

### Example 1: Vector Construction Cloning of AAV Production Plasmids

### Generation of Monocistronic Self-Complementary AAV Production Plasmids

Briefly, the AAV production plasmids, scAAV-LP1-GCH1 (pAA009) and scAAV-LP1-TH (pAA010) (SEQ ID NO: 34), used to produce the double-stranded rAAV2/8-LP1-GCH1 and rAAV2/8-LP1-tTH, respectively, were constructed by digesting scAAV-LP1-hFIXco with XbaI and SpeI and ligating it with either the GCH1 or tTH NheI/NheI PCR fragment isolated from pLA100 (ssAAV-SYN-GCH1-SYN-TH-WPRE) and pLA109 (ssAAV-SYN-GCH1-SYN-tTH), respectively. The scAAV-LP1-GCH1 (pAA009) (SEQ ID NO: 35) and scAAV-LP1-tTH (pAA010) (SEQ ID NO: 34) vectors were constructed as follows: The 992 bp GHC1 fragment of pLA100 (ssAAV-SYN-GCH1-SYN-TH) was amplified using primers AA16 (forward primer containing NheI site, 5′-ccaagctagcATGGAGAAGGGCCCTGTG-3′, SEQ ID NO: 42) and AA17 (reverse primer containing NheI site, 5′-ccaagctagcGGTCGACTAAAAAACCTCC-3′, SEQ ID NO: 43) at a concentration of 0.75 pmol/μl with 25 ng template DNA, 200 μM dNTPs (NEB) and GoTaq Polymerase (Promega) in appropriate buffer. Conditions of the PCR amplifications were as follows: 95° C. (2 min), followed by 30 cycles of 95° C. (30 s)/65° C. (30 s)/72° C. (30 s), and a final extension at 72° C. for 5 minutes. The 1858 bp tTH-WPRE fragment of pLA109 (ssAAV-SYN-GCH1-SYN-tTH) was amplified using primers AA33 (forward primer containing NheI site, 5′-CCAAgctagcATGAGCCCCGCGGGGCCCAAG-3′, SEQ ID NO: 44) and AA34 (reverse primer containing NheI site, 5′-CCAAgctagcGGGGGATCTTCGATGCTAGAC-3′, SEQ ID NO: 45) at a concentration of 0.4 pmol/μl with 25 ng DNA, 200 μM dNTPs (NEB) and Phusion Polymerase (Thermo Scientific) in appropriate buffer. Conditions of the PCR amplifications were as follows: 98° C. (30 s), followed by 30 cycles of 98° C. (10 s)/63° C. (30 s)/72° C. (1 min), and a final extension at 72° C. for 10 minutes. The PCR products (inserts) were digested with NheI for 3 h at 37° C. and plasmid scAAV-LP1-hFIXco (vector) (SEQ ID NO: 43) was digested with XbaI/SpeI for 3 h at 37° C. in order to remove the hFIXco gene. Digestions were analysed by gel electrophoresis after 1 h migration at 100V in a 1% agarose gel and visualised on a UV trans-illuminator. Fragments (GCH1 insert: 992 bp; tTH insert: 1858; vector: 3525 bp) were cut out from the gel using a scalpel blade and purified from the gel using the QIAquick Gel Extraction Kit (Qiagen). Vector was ligated overnight at 16° C. with either insert and transformed into SURE bacteria. Colonies were picked and analysed by XcmI digestion to check the presence of either GCH1 or tTH PCR fragments and subsequently sent for sequencing to confirm that each construct contained the expected sequence.

The final transgene constructs are two plasmids for dsAAV production containing either the human GCH1 or the truncated human TH gene (e.g. SEQ ID NO: 40) under the control of the liver-specific LP1 enhancer/promoter, all flanked by AAV2 ITRs.

Replacement of LP1 Promoter by HLP in pAA009 and pAA010

The AAV production plasmids, scAAV-HLP-GCH1 (pAA011) (SEQ ID NO: 31) and scAAV-HLP-tTH (pAA016) (SEQ ID NO: 32) were used to produce the double-stranded rAAV2/8-HLP-GCH1 and rAAV2/8-HLP-tTH, respectively. Briefly, pAA011 (SEQ ID NO: 35) was constructed by amplifying the HLP promoter from AV-HLP-codop-hFVIII-V3 (gently provided by Amit Nathwani) with the primer set AA43/AA44 (5′ CCAATGGCCAACTCCATCACTAGGGGTTCCTTCTAGATGTTTGCTGCTTGCAATGT TTGC 3′/5′ CCAAGAATTCGCTAGCGATTCACTGTCCCAGGTCAGTG 3′, SEQ ID NO: 46 and SEQ ID NO: 47, respectively) and cloning it with MscI and EcoRI into pAA009 (SEQ ID NO: 35) in place of the LP1 promoter. pAA016 (SEQ ID NO: 32) was generated by amplifying the fragment HLP-tTH by overlapping PCR. Primer pairs AA57/AA67 (5′ CCAAGCTAGC TGT TTG CTG CTT GCA ATG TTT GC 3′/5′ GATCCTTGCTACGAGCTTGAATGATTCACTGTCCCAGGTCAGT 3′, SEQ ID NO: 48 and SEQ ID NO: 49, respectively) and AA68/RmuscTHext2 (5′ ACTGACCTGGGACAGTGAATCATTCAAGCTCGTAGCAAGGATC 3′/5′ AAAgctagcTTCGATGCTAGACGATCCAG 3′, SEQ ID NO: 50 and SEQ ID NO: 51, respectively) were used to generate fragments HLP and tTH, respectively, containing overlapping sequences. HLP was fused to tTH by an overlapping PCR using primers AA57/AA67 and subcloned into pcDNA3.1(+) using the NheI restriction endonuclease, thereby generating pAA015. At last, the HLP-tTH fragment was cut out from pAA015 using NheI and ligated into the vector pAV-LP1-hFIXco between the restriction sites NheI and SpeI, thereby generating pAA016 (SEQ ID NO: 32).

The 298 bp HLP fragment was amplified in a 20 μl PCR reaction using 20 ng template DNA, 200 μM dNTPs (NEB) and Phision High Fidelity Polymerase (Fischer Scientific) in appropriate buffer. Conditions of the PCR amplification was as follows: 98° C. (30 s), followed by 30 cycles of 98° C. (10 s)/65° C. (15 s)/72° C. (60 s), and a final extension at 72° C. for 10 minutes.

The 2.1 kb HLP-tTH fragment generated by overlapping PCR was amplified in a 20 μl PCR reaction using 45 ng of HLP template DNA and 306 ng tTH template DNA, each generated previously by PCR. 200 μM dNTPs (NEB) and Phision High Fidelity Polymerase (Fischer Scientific) were used in appropriate buffer and the cycling conditions of the PCR amplification was as follows: 98° C. (30 s), followed by 30 cycles of 98° C. (10 s)/60° C. (15 s)/72° C. (60 s), and a final extension at 72° C. for 10 minutes.

### Generation of Bicistronic Single-Stranded AAV Production Plasmid

AAV production plasmid ssAAV-LP11-GCH1-LP1-tTH (pAA019) (SEQ ID NO: 33) was used to generate the single-stranded rAAV2/8-LP1-GCH1-LP1-tTH and its recombinant by-product rAAV2/8-LP1-tTH. Briefly, the expression cassettes LP1-GCH1-LP1-tTH-WPRE were subcloned into pBluescript II SK(+) making pAA018 prior to cloning in the AAV backbone pSUB201 containing ITRs, thereby forming pAA019 (SEQ ID NO: 33). The promoter LP1 was amplified with primers AA01/AA02 using 12.5 ng scAAV-LP1-hFIXco as a template and cloned into pTRUF11 using BIpI and SbfI restriction sites, thereby generating pAA001. Next, the GCH1 gene was amplified with primers AA03/AA004 using 27 ng pAAV-Syn-GCH1-Syn-TH as a template and subsequently cloned into pAA001 using the SbfI and Tth111I sites, thereby forming pAA002. Next, the LP1-GCH1 fragment was amplified from pAA002 using the primer pair AA37/AA38, which contained overhangs with the XbaI/BlpI and XbaI/SphI/BstBI/Tth111I restriction sites, respectively to allow the construction of a modular vector. The LP1-GCH1 fragment was ligated into the AAV backbone pSub201 through the XbaI restriction site, thereby forming pAA003. To avoid cloning difficulties due to the presence of ITRs in the backbone, the LP1-GCH1 was transferred to the cloning vector pUC18 through the XbaI site, thereby forming pAA004. The second LP1 promoter was added by amplifying it from pAA010 with primer pairs AA006/AA07 and cloning it into pAA004 using BstBI and Tth111I restriction sites, thereby forming pAA005. In order to add the tTH gene to the construct, the LP1-GCH1-LP1 fragment had to be changed into the backbone pBluescript II SK(+) due to the presence of an extra SphI site in pUC18. This was done using the XbaI sites in pAA005 and after ligation into pBluescript II SK(+) the new construct was named pAA006. Next, the tTH-WPRE fragment was amplified from pLA109 (AAV-Syn-GCH1-Syn-tTH) using primer pair AA53/AA65 and 50 ng of template. The tTH gene was inserted into pAA006 through the restriction sites SphI and BstBI, thereby forming pAA018. After sequencing of pAA018, a mutation on the Tth111I site was found and this was fixed by recloning the GCH1-LP1 sequence. Here, a new primer set was designed to add a BglII restriction site immediately downstream of the Ttth111I site and to allow the incorporation of the exact same GCH1 kozak sequence as in pLA100 and pLA109. Primer pairs AA73/AA84 and AA85/AAA07 were used to amplify the new GCH1 sequence and the second LP1 promoter, respectively. An overlapping PCR with primer pair AA73/AA07 was done to fuse GCH1-LP1, which was subsequently cloned into pAA017 using restriction sites SbfI and BstBI, thereby forming pAA018. Finally, the whole bicistronic LP1-GCH1-LP1-tTH expression cassette was transferred back to the AAV backbone pSub201 to allow recombinant AAV production and named pAA019 (SEQ ID NO: 33).

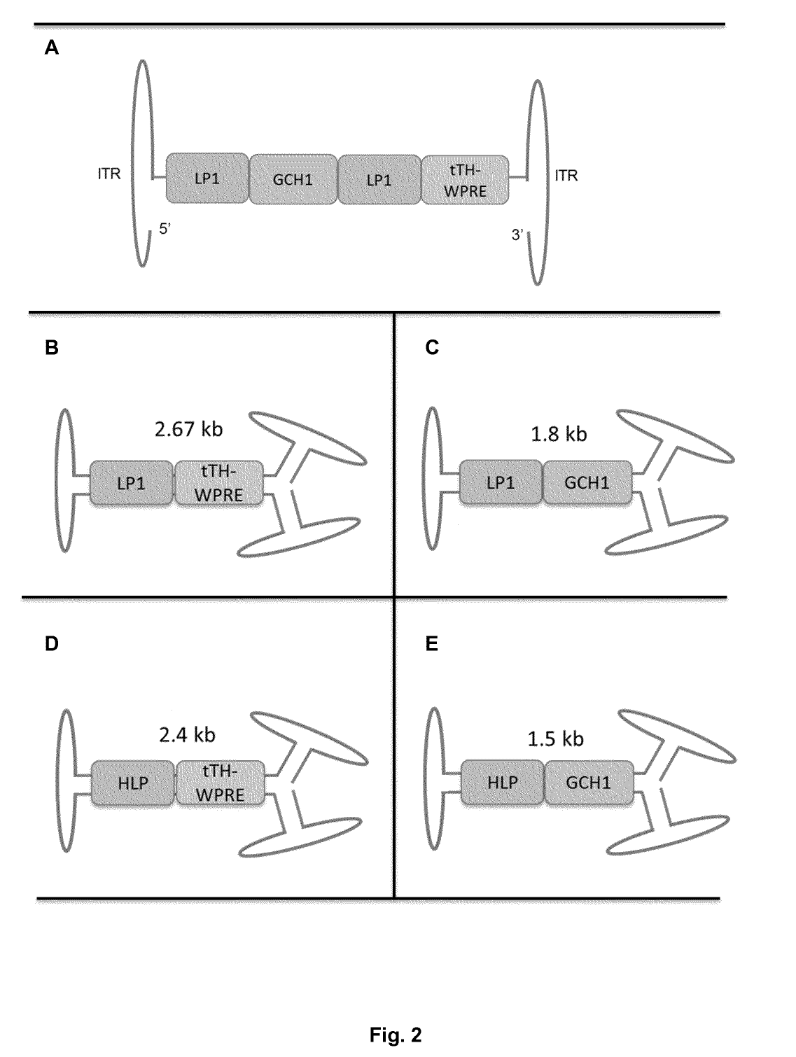

Monocistronic self-complementary AAV-HLP-tTH was generated by fusing the HLP promoter to the tTH gene by overlapping PCR. The HLP sequence was amplified from AV-HLP-codop-hFVIII-V3 (a plasmid provided by Amit Nathwani's lab). The sequence of the tTH is the sequence of TH from with the N terminus 160 amino acids have been truncated (e.g. SEQ ID NO: 40) to remove the key serine phosphorylation sites otherwise involved in enabling the feedback inhibition of TH by dopamine or L-DOPA. Once HLP and tTH were amplified, they were fused by overlapping PCR and subcloned it into pcDNA3.1(+) using the NheI restriction site. After the quality control digestions and sequencing, the expression cassette HLP-tTH was cloned an AAV self-complementary backbone provided by Amit Nathwani (FIG. 2).

Monocistronic self-complementary AAV-HLP-GCH was generated by amplifying the GCH1 gene from pGPT001 (SYN-GCH1-SYN-TH) and cloning it into a self-complementary AAV backbone pAV-LP1-hFIXco (SEQ ID NO: 36) (provided by Amit Nathwani), thereby generating AAV-LP1-GCH1. In a second step, the HLP promoter sequence was amplified from AV-HLP-codop-hFVIII-V3 (SEQ ID NO: 37) and ligated into scAAV-LP1-GCH1, thereby replacing the LP1 by HLP to form scAAV-HLP-GCH1 (FIG. 2).

Bicistronic single-stranded AAV-LP1-GCH1-LP1-tTH was generated using the AAV plasmid pSUB201 as a backbone. Optimal restriction sites flanked by the ITRs were identified in order to produce a modular vector in which each element (gene or promoter) could be easily removed or replaced. Both LP1 sequences were amplified by PCR from pAV-LP1-hFIXco and cloned into pSUB201. GCH1 and tTH were amplified from the pre-existing bicistronic vector used for the brain study (SYN-GCH1-SYN-tTH) and cloned into pSUB201 to form ssAAV-LP1-GCH1-LP1-tTH. The chronology of the cloning was first LP1-GCH1-second LP1-tTH (FIG. 2).

Other vectors were constructed by conventional methods known in the art. Sequences of interest were subcloned into vectors by restriction, ligation and Gibson assembly.

AAV vectors were prepared by triple transfection in adherent HEK293 cells, and optionally concentrated by iodixanol gradient centrifugation.

### Example 2: L-DOPA Inhibition

The dosing regime has been designed to assess the ability of Adeno-associated virus vectors carrying the gene with GTP cyclohydrolase 1 and/or tyrosine hydroxylase (AAV2/8 GCH1 or AAV2/8 tTH, respectively), to induce the production of L-DOPA in the liver of Parkinson's disease (PD) patients.

Two studies were performed. In the first study 18 CD1 mice were randomly allocated to 3 groups of 6 animals. On day 1 animals were treated as indicated in the table below:

| | | | Dose |
|---|---|---|---|
| Group | Vector (AAV2/8) | Animals | (vg/mouse) |
| 1 | — | 6 | — |
| 2 | scLP1-GCH1 | 6 | 3.51 × 10¹⁰ |
| | scLP1-tTH | | 3.51 × 10¹⁰ |
| 3 | scLP1-tTH | 6 | 7.02 × 10¹⁰ |

The vectors, scLP1-GCH1 (SEQ ID NO:35) and scLP1-tTH (SEQ ID NO:34) were prepared as described in Example 1. The vectors were administered by bolus intravenous (tail vein) injection.

In the second study 4 CD1 mice were randomly allocated to 2 groups of 2 animals. On day 1 animals were treated as indicated in the table below:

| | Vector | | Dose |
|---|---|---|---|
| Group | (AAV2/8) | Animals | (vg/mouse) |
| 1 | scHLP-tTH | 2 | 3.60 × 10¹² |
| 2 | scHLP-GCH1 | 2 | 1.80 × 10¹² |
| | scHLP-tTH | | 1.80 × 10¹² |

The vectors, scHLP-GCH1 (SEQ ID NO:31) and scHLP-tTH (SEQ ID NO:32) were prepared as described in Example 1.

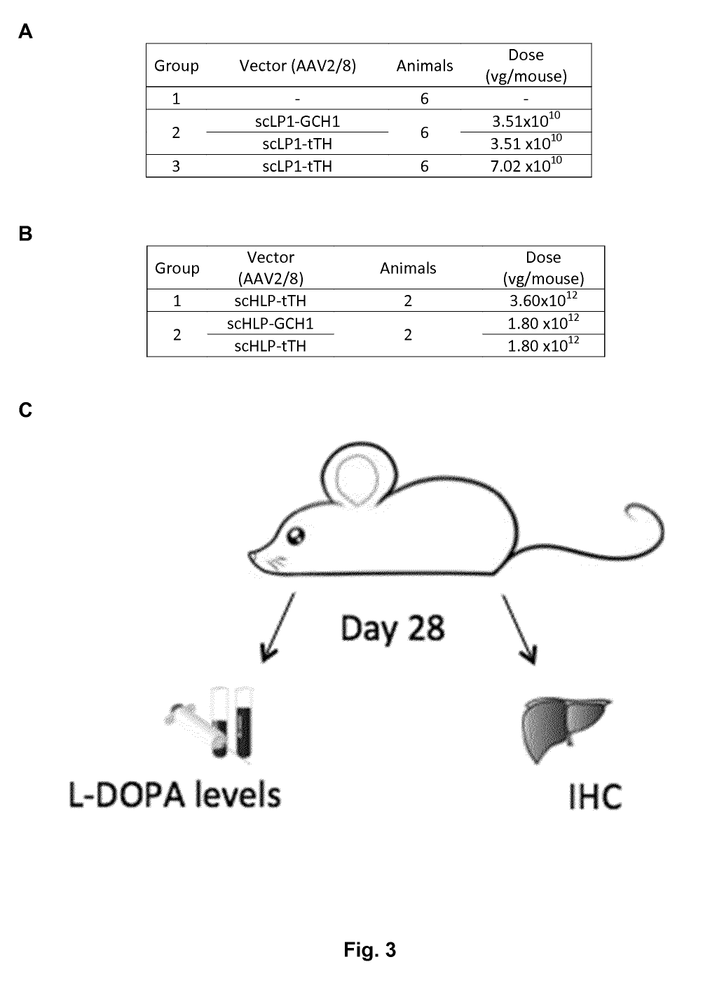

Both in the first and second study the vectors were administered by bolus intravenous (tail vein) injection (FIG. 3).

The mice were observed without further experimentation for 28 days. No adverse events were noted. On day 28, one hour before sacrifice, the mice were dosed with benserazide 10 mg/kg by intraperitoneal injection and with a low dose of entacapone by intraperitoneal injection. The nominal injected dose of entacapone was 30 mg/kg (FIG. 3).

At the time of sacrifice blood samples were obtained by cardiac puncture, after which animals were perfused with PBS followed by PFA and the liver was harvested.

Blood was collected into vials containing heparin and stored on ice until the last animal was sacrificed, then spun at 4 degrees with subsequent freezing of the plasma at −70° C. in the absence of antioxidants.

L-DOPA was assayed by ABS Laboratories Ltd, BioPark, Broadwater Road, Welwyn Garden City, Hertfordshire, AL7 3AX, United Kingdom using a validated method and conducted according to the European Medicines Agency bioanalytical guidelines with appropriate calibration standards and quality control samples run in duplicate with the samples and deuterated internal standardization.

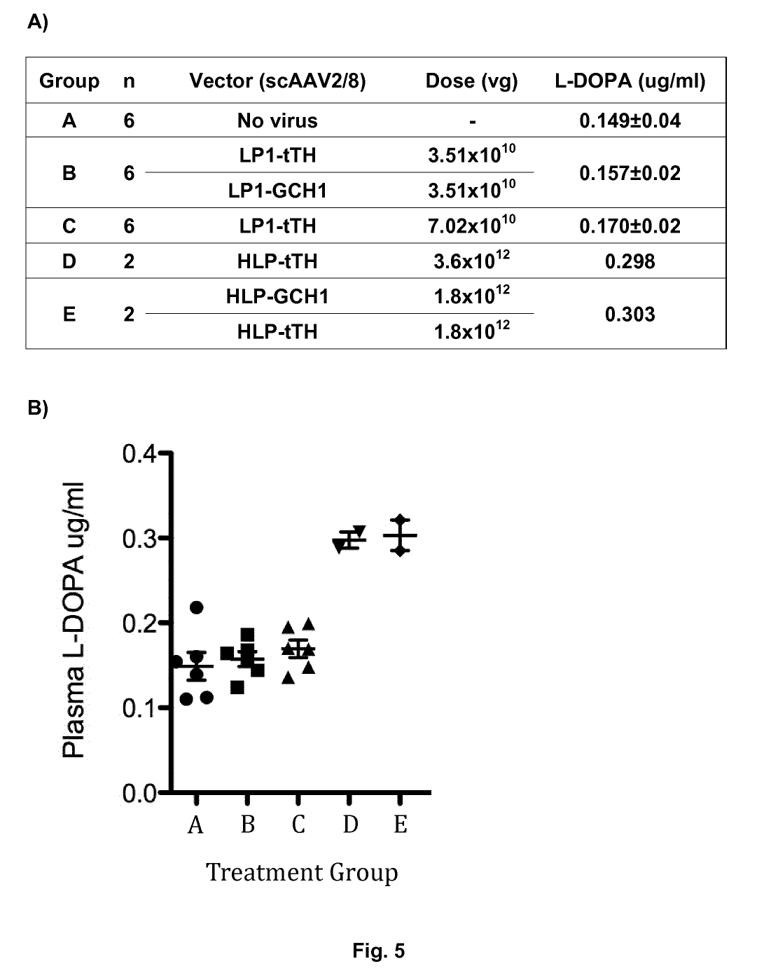

The results are shown in FIG. 5, where the groups A, B and C are from the first animal study, whereas the groups D and E are from the second animal study.

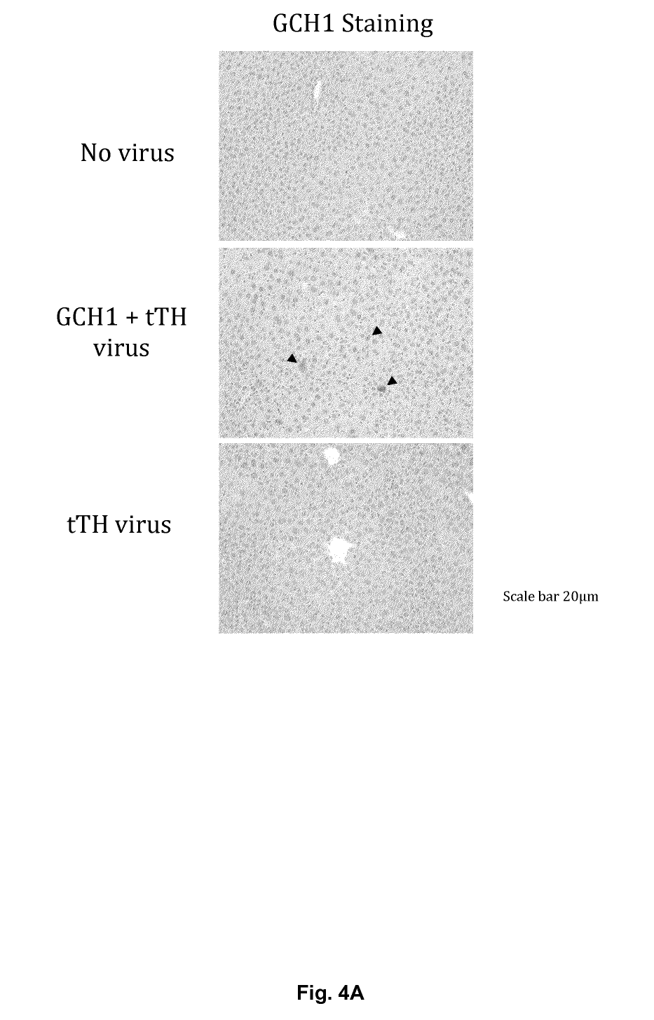

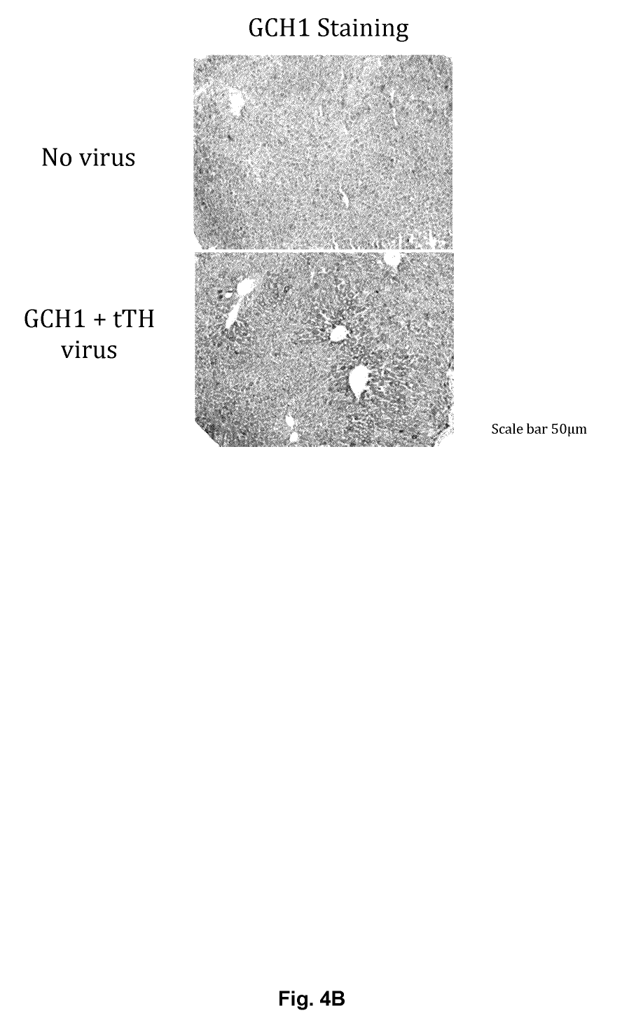

Liver was fixed in PFA then embedded in paraffin, mounted on slides and analysed. The liver section were analysed for GCH1 expression using a GCH1 specific antibody. Useful GCH1 specific antibodies are commercially available and include e.g. the mouse IgG MCA3138Z, Serotec, Oxford, UK, which may be used at 1:2000 AbD. UK. The results obtained in the first animal study are shown in FIG. 4a. The transduction was determined to be <1%. The results obtained in the second animal study are shown in FIG. 4b. The transduction was determined to be ˜25%.

Expression of TH may be determined using a number of anti-Tyrosine Hydroxylase antibodies including those produced by Pel Freez and Abcam.

Dilutions useful for the IHC with:

Pelfreez Anti-Tyrosine Hydroxylase rabbit polyclonal antibody. 1:750
Abcam Anti-Tyrosine Hydroxylase rabbit monoclonal [EP1532Y]-1:1000

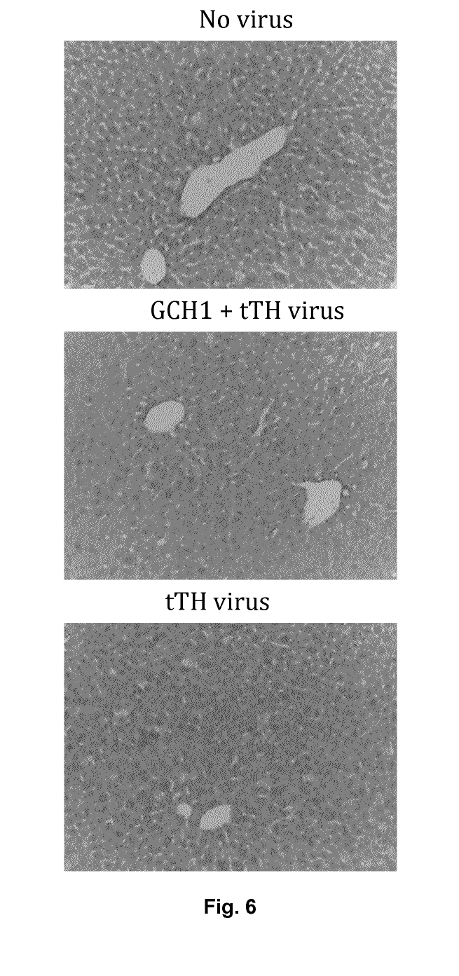

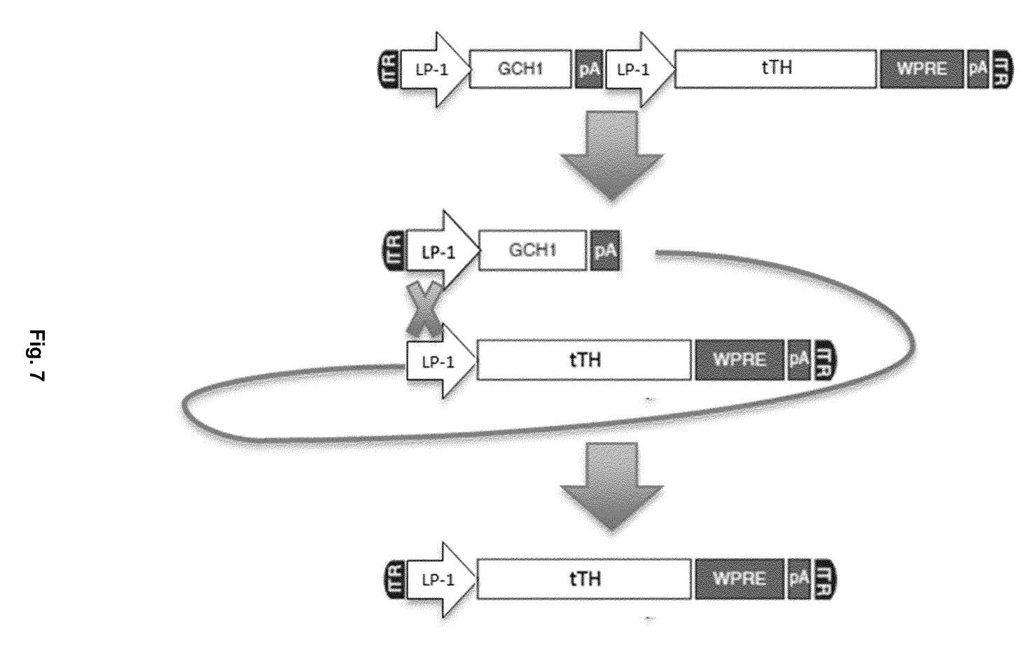

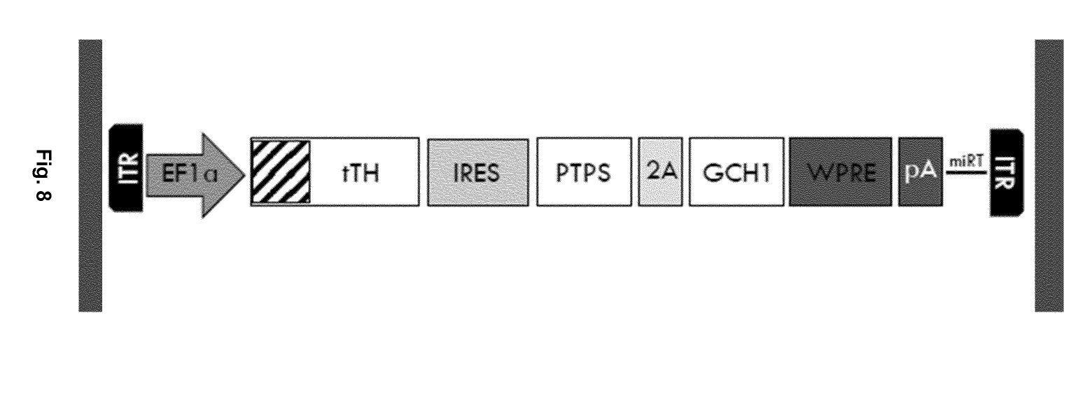

The liver sections were also stained with hematoxylin and eosin using standard procedures. The hematoxylin and eosin stain shows no signs of tissue damage or leukocyte infiltration (see FIG. 6)

### CONCLUSION

In the first animal study a low dose of vector (7.02×10¹⁰ vg/mouse) was administrated. As shown by liver immunohistochemistry this resulted in a transduction of <1% (see FIG. 4a). In the second animal study a higher dose of vector (3.6×10¹² vg/mouse) was administered and transduction was markedly higher, namely ˜25% (see FIG. 4b). Hiroyuki Nakai et al J. Virol. 2005, 79(1):214 has suggested that dose (vg/mouse) of AAV8 vectors needs to exceed 2E12 to achieve >70% tranduction. Consisting with this, the higher dose resulted in enhanced transduction.

HLP is a short liver-specific promoter equally strong to LP1 (McIntosh J et al, Blood. 2013 Apr. 25; 121(17):3335-44). Internal controls on L-DOPA assay confirmed consistent sensitivity across animal study 1 and 2

As shown in FIG. 5 systemic L-DOPA levels in mice of groups 2 and 3 in the first animal study (denoted B and C, respectively) are slightly higher than the level in the control. However, the systemic L-DOPA level in mice of both groups 1 and 2 of the second animal study (denoted D and E, respectively) were markedly higher than the control. The difference in systemic L-DOPA levels observed in the two studies is believed to be caused by the difference in dose resulting in different transduction efficiency.

In further studies two or three doses of benserazide and entacapone or tolcapone will be administered during the 8 hours prior to collection of blood for L-DOPA assay and plasma will be stored in the presence of antioxidant (25% w/v sodium metabisulphite in water) prior to assay.

### Example 3: Vector Synthesis

A series of vectors are synthesised to transfect and transduce peripheral tissues to secrete L-dopa at a steady rate into the peripheral circulation from which it can cross the blood brain barrier and be used as a prodrug for the synthesis of dopamine. These include vectors with the following configurations or element:
The vector(s) include a nucleic acid sequence encoding a human tyrosine hydroxylase isoform, wherein the nucleic acid sequence is configured as a self-complementary genome.
In one embodiment the nucleic acid sequence is truncated to encode an N-terminally truncated tyrosine hydroxylase enzyme lacking the about 160 N-terminally amino acids of the functional enzyme (SEQ ID NO: 15) or (SEQ ID NO: 40). The N-terminally truncated enzyme is functional but less prone to feedback inhibition by the product(s) of the reaction catalyzed by the enzyme. Accordingly an increased production to a therapeutically effective level of the desired L-DOPA product is achieved.
In one embodiment the construct does not utilise a self-complementary genome.
Vector construct are being produced with a variety of AAV serotypes targeting liver and muscle. These include serotypes 8, 5, 2 and 7 for liver and 5, 1, 6 and 2 for muscle.
Vector constructs include a variety of tissue specific promoters such as LP1 for liver.
A model vector sequence is provided by the attachment below from a paper (attached) by Nathwani et al. In the case of our vector would have a similar sequence to the Nathwani et all Factor IX genome but with a self-complementary TH code inserted in place of the FIX code.

### Example 4: Expression of GCH and TH in the Liver

On day 1, the mice are receiving either: a bolus intravenous (tail vein) injection of 0.15 ml bicistronic vector preparation (ssAAV2/8-LP1-GCH1-LP1-truncated-TH) 3.60E+12 vg/mouse (this preparation including a proportion of monocistronic ssLP1-tTH formed by homologous recombination); a bolus intravenous (tail vein) injection of 0.15 ml vehicle preparation; or 10 mg/kg oral L-DOPA.

The mice are observed for 10-15 days before sacrifice and collection of the plasma, as described in example 3.

Analysis of the expression of GCH1 and TH in the liver is performed by qPCR.

Immunohistochemical analysis is performed as described in example 3 to show expression of GCH1 in liver sections derived from the mice having received the bicistronic vector. Expression of GCH1 may be used as a marker of vector transfection.

Western Blot analysis is performed to show that GCH1 is only expressed in the livers of mice having received a bolus intravenous injection of vector.

### Example 5: Synthesis of L-DOPA in the Liver

L-DOPA levels are determined in EDTA plasma by precipitating the proteins in the plasma with 0.4M perchloric acid. After removal of the precipitated proteins by centrifugation, a portion of the perchloric acid layer is transferred to a 96-well plate and diluted with 0.1% formic acid. The L-DOPA (I) and its stable isotopically labelled internal standard L-DOPA-d₃ (II) are analysed by LC-MS/MS.

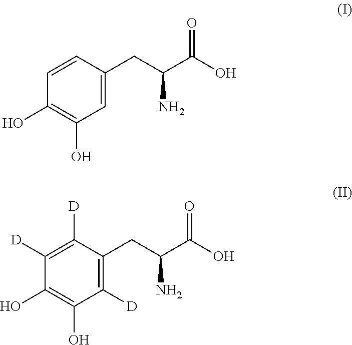 [SMILES: N[C@@H](Cc1ccc(O)c(O)c1)C(=O)O ; [2H]c1c([2H])c(C[C@H](N)C(=O)O)c([2H])c(O)c1O]

As L-DOPA is unstable in plasma, all plasma containing L-DOPA is stabilised by the addition of 1% sodium metabisulphite and stored frozen at a nominal temperature of −80° C. Calibration standards are prepared at 0 (blank), 0.020, 0.050, 0.100, 0.250, 1.00, 2.50, 5.00 and 10.0 μg/mL and quality control samples (QCs) at 0.060, 0.800 and 8.00 μg/mL.

The analysis is performed using a 0.1% formic acid acetonitrile gradient on an ACE AQ 50 mm×3 mm liquid chromatography column using an Agilent 1100 series binary pump and a CTC Analytics™ CTC HTS-xt PAL autosampler. The mass spectrometric analysis is performed using an Applied Biosystems™ API4000 fitted with a Turbolonspray™ ion source. The multiple reaction ions monitored (MRM) for L-DOPA and L-DOPA-d₃ were m/z 198.2→152.1 and 201.2→155.1, respectively. Calibration curves are fitted using a linear regression weighted 1/x2.

### Example 6: Screening

Each vector prepared as described herein above is injected into the tail vein or hindlimb muscle bulk of a group of mice (approximately 6 per group). The mice are observed for 2-6 weeks post dosing. Peripheral blood is collected and assayed for L-dopa.
Animals receive concomitant dosing with tetrahydrobioterin (oral or intraperitoneal) or with an AAV vector transducing GCH1 and/or PTPS production in liver or muscle in order to provide this cofactor necessary for L-dopa synthesis.
Animals receive systemically administered (oral or intraperitoneal) decarboxylase inhibitor (e.g. benserazine) and catechol-O-methyltransferase (COMT) inhibitor to limit catabolism of L-DOPA. These are administered for a minimum of 24 hours before samples are collected to assess peripheral L-DOPA levels.
A control group of animals is treated in the same manner but without injection of vector. This groups serves as control group against which to compare L-DOPA levels from the vector treated animals.
Monocistronic, bicistronic or tricistronic vectors, plasmids or expression systems expressing different ratios of TH, GCH1 and PTPS may be compared to achieve optimal L-DOPA production.
Different ratios of vectors (each expressing one or more genes) may be compared to achieve optimal L-DOPA production.

### Example 7: Preclinical

The vectors producing the highest peripheral L-dopa levels are tested in acute and chronic studies in rodents and non-human primates to demonstrate sustained secretion of L-dopa at therapeutically relevant levels and to demonstrate acceptable tolerance and safety.
In acute studies the vector is injected either intramuscularly or intravenously (into a peripheral vein or directly into the portal vein) of rodents and non-human primates. The animals are observed for 28 days post injection. Observations include weight, food consumption, observation of any clinical signs or symptoms, full blood count, urea and electrolytes, liver function tests, and measurement of creatine phosphokinase. Following necropsy tissue will be examined for evidence of any histopathological abnormality and biodistribution of the vector will be assessed.
In chronic studies the vector is injected either intramuscularly or intravenously (into a peripheral vein or directly into the portal vein) of rodents and non-human primates. The animals are observed for six to 12 months post injection. Observations include weight, food consumption, observation of any clinical signs or symptoms, full blood count, urea and electrolytes, liver function tests, and measurement of creatine phosphokinase. Following necropsy tissue will be examined for evidence of any histopathological abnormality and biodistribution of the vector will be assessed.
Additional preclinical studies will include mutagenicity test, carcinogenicity tests and other tests necessary to enable clinical studies (e.g. assessment of effect of vector or vector produced product on cardiac QT interval)

### Example 8: Clinical

Subject to satisfactory outcomes of the above studies clinical studies are designed based on the optimally performing vector(s) using either IM, IV, direction infusion into the portal vein or isolated limb perfusion.
Clinical studies will include detailed assessment of the pharmacokinetics of L-DOPA in treated patients with and without concomitant administration of an (oral or intraperitoneal) decarboxylase inhibitor (e.g. benserazine) and catechol-O-methyltransferase (COMT) inhibitor and without administration of BH4 or oral L-DOPA.
Clinical studies will assess acute L-DOPA production (approximately 4 to 8 weeks following injection of the vector) and chronic L-DOPA production at time points including 3, 6, 12, 18 and 24 months after injection of vector.
Clinical studies will include assessment of the acute and chronic safety and

### Example 9: Overview of Sequences

SEQ ID NO: 1: GTP cyclohydrolase 1 (human)

SEQ ID NO: 2: GTP cyclohydrolase 1 Isoform GCH-2 (human)

SEQ ID NO: 3: GTP cyclohydrolase 1 Isoform GCH-3 (human)

SEQ ID NO: 4: GTP cyclohydrolase 1 Isoform GCH-4 (human)

SEQ ID NO: 5: GTP cyclohydrolase 1 (rat)

SEQ ID NO: 6: GTP cyclohydrolase 1 (mouse)

SEQ ID NO: 7: Tyrosine 3-hydroxylase (human)

SEQ ID NO: 8: Tyrosine 3-monooxygenase (human)

SEQ ID NO: 9: Tyrosine hydroxylase (human)

SEQ ID NO: 10: Tyrosine hydroxylase (human)

SEQ ID NO: 11: Tyrosine 3-monooxygenase (human)

SEQ ID NO: 12: Truncated Tyrosine hydroxylase, TH (corresponding to catalytic domain; human)

SEQ ID NO: 13: TH mutated at ser40

SEQ ID NO: 14: SEQ ID NO: 14: TH mutated at Ser19+Ser40

SEQ ID NO: 15: SEQ ID NO: 15: TH mutated at Ser19+Ser31+Ser40

SEQ ID NO: 16: SEQ ID NO: 16: Tyrosine 3-hydroxylase (rat)

SEQ ID NO: 17: Tyrosine 3-hydroxylase (mouse)

SEQ ID NO: 18: Adeno-associated virus 2 left terminal nucleotide sequence

SEQ ID NO: 19: Adeno-associated virus 2 right terminal nucleotide sequence

SEQ ID NO: 20: Homo sapiens GTP cyclohydrolase 1 (GCH1), transcript variant 1

SEQ ID NO: 21: Simian virus 40 early poly-adenylation nucleotide sequence

SEQ ID NO: 22: Simian virus 40 late poly-adenylation nucleotide sequence

SEQ ID NO: 23: Homo sapiens tyrosine hydroxylase (TH), transcript variant 2 nucleotide sequence

SEQ ID NO: 24: Truncated TH, nucleotide sequence encoding catalytic domain

SEQ ID NO: 25: TH mutated at ser40, nucleotide sequence

SEQ ID NO: 26: TH mutated as ser19 and ser40, nucleotide sequence

SEQ ID NO: 27: TH mutated as ser19, ser31 and ser40, nucleotide sequence

SEQ ID NO: 28: Woodchuck hepatitis B virus (WHV8) post-transcriptional regulatory element nucleotide sequence

SEQ ID NO: 29: Mutated Woodchuck hepatitis B virus (WHV8) post-transcriptional regulatory element nucleotide sequence

SEQ ID NO: 30: Nucleotide sequence encoding GCH-1

SEQ ID NO: 31: pAA011-scAAV-HLP-GCH1

SEQ ID NO: 32: pAA016-scAAV-HLP-tTH

SEQ ID NO: 33: pAAo19-scAAV-LP1-GCH1-LP1-tTH

SEQ ID NO: 34: pAA010 scAAV-LP1-tTH

SEQ ID NO: 35: pAA009 scAAV-LP1-GCH1

SEQ ID NO: 36: scAAV-LP1-hFIXco

SEQ ID NO: 37: pAV HLP FVIII V3 kan

SEQ ID NO: 38: Hybrid liver-specific promoter (HLP)

SEQ ID NO: 39: Liver promoter/enhancer 1 (LP1)

SEQ ID NO: 40: tTH=truncated Tyrosine Hydroxylase

SEQ ID NO: 41: PTPS=6-pyruvoyltetrahydropterin synthase

SEQ ID NO: 42: Primer AA16

SEQ ID NO: 43: Primer AA17

SEQ ID NO: 44: Primer AA33

SEQ ID NO: 45: Primer AA34

SEQ ID NO: 46: Primer AA43

SEQ ID NO: 47: Primer AA44

SEQ ID NO: 48: Primer AA57

SEQ ID NO: 49: Primer AA67

SEQ ID NO: 50: Primer AA68

SEQ ID NO: 51: Primer RmiscTHext2

SEQ ID NO: 52: Monocistronic delivery plasmid TH

SEQ ID NO: 53: Bicistronic delivery plasmid GCH1 PTPS

SEQ ID NO: 1: GTP cyclohydrolase 1 (human)
>sp|P30793|GCH1_HUMAN GTP cyclohydrolase 1 OS = Homo sapiens GN = GCH1 PE = 1 SV = 1
EC = 3.5.4.16
Alternative name(s):
GTP cyclohydrolase I
Short names = GTP-CH-Ior GCH-1 or GCH1 or GCH1
Organism: Homo sapiens (Human)
http://www.uniprot.org/uniprot/P30793
MEKGPVRAPAEKPRGARCSNGFPERDPPRPGPSRPAEKPPRPEAKSAQPADGWKGERPRSEEDNELNLPNLAAAYSSILSSLGENPQRQG
LLKTPWRAASAMQFFTKGYQETISDVLNDAIFDEDHDEMVIVKDIDMFSMCEHHLVPFVGKVHIGYLPNKQVLGLSKLARIVEIYSRRLQ
VQERLTKQIAVAITEALRPAGVGVVVEATHMCMVMRGVQKMNSKTVTSTMLGVFREDPKTREEFLTLIRS
SEQ ID NO: 2: GTP cyclohydrolase 1 Isoform GCH-2 (human)
>sp|P30793-2|GCH1_HUMAN Isoform GCH-2 of GTP cyclohydrolase 1 OS = Homo sapiens GN = GCH1
MEKGPVRAPAEKPRGARCSNGFPERDPPRPGPSRPAEKPPRPEAKSAQPADGWKGERPRSEEDNELNLPNLAAAYSSILSSLGENPQRQG
LLKTPWRAASAMQFFTKGYQETISDVLNDAIFDEDHDEMVIVKDIDMFSMCEHHLVPFVGKVHIGYLPNKQVLGLSKLARIVEIYSRRLQ
VQERLTKQIAVAITEALRPAGVGVVVEATSAEP
SEQ ID NO: 3: GTP cyclohydrolase 1 Isoform GCH-3 (human)
>sp|P30793-3|GCH1_HUMAN Isoform GCH-3 of GTP cyclohydrolase 1 OS = Homo sapiens
GN = GCH1
MEKGPVRAPAEKPRGARCSNGFPERDPPRPGPSRPAEKPPRPEAKSAQPADGWKGERPRSEEDNELNLPNLAAAYSSILSSLGENPQRQG
LLKTPWRAASAMQFFTKGYQETISDVLNDAIFDEDHDEMVIVKDIDMFSMCEHHLVPFVGKVHIGYLPNKQVLGLSKLARIVEIYSRRLQ
VQERLTKQIAVAITEALRPAGVGVVVEAT
SEQ ID NO: 4: GTP cyclohydrolase 1 Isoform GCH-4 (human)
>sp|P30793-4|GCH1_HUMAN Isoform GCH-4 of GTP cyclohydrolase 1 OS = Homo sapiens GN = GCH1
MEKGPVRAPAEKPRGARCSNGFPERDPPRPGPSRPAEKPPRPEAKSAQPADGWKGERPRSEEDNELNLPNLAAAYSSILSSLGENPQRQG
LLKTPWRAASAMQFFTKGYQETISDVLNDAIFDEDHDEMVIVKDIDMFSMCEHHLVPFVGKVHIGYLPNKQVLGLSKLARIVEIYSRRLQ
VQERLTKQIAVAITEALRPAGVGVVVEATKSNKYNKGLSPLLSSCHLFVAILK
SEQ ID NO: 5: GTP cyclohydrolase 1 (rat)
>sp|P22288|GCH1_RAT GTP cyclohydrolase 1 OS = Rattus norvegicus GN = Gch1 PE = 1 SV = 1
MEKPRGVRCTNGFPERELPRPGASRPAEKSRPPEAKGAQPADAWKAGRPRSEEDNELNLPNLAAAYSSILRSLGEDPQRQGLLKTPWRAA
TAMQFFTKGYQETISDVLNDAIFDEDHDEMVIVKDIDMFSMCEHHLVPFVGRVHIGYLPNKQVLGLSKLARIVEIYSRRLQVQERLTKQI
AVAITEALQPAGVGVVIEATHMCMVMRGVQKMNSKTVTSTMLGVFREDPKTREEFLTLIRS
SEQ ID NO: 6: GTP cyclohydrolase 1 (mouse)
>sp|Q05915|GCH1_MOUSE GTP cyclohydrolase 1 OS = Mus musculus GN = Gch1 PE = 2 SV = 1
MEKPRGVRCTNGFSERELPRPGASPPAEKSRPPEAKGAQPADAWKAGRHRSEEENQVNLPKLAAAYSSILLSLGEDPQRQGLLKTPWRAA
TAMQYFTKGYQETISDVLNDAIFDEDHDEMVIVKDIDMFSMCEHHLVPFVGRVHIGYLPNKQVLGLSKLARIVEIYSRRLQVQERLTKQI
AVAITEALQPAGVGVVIEATHMCMVMRGVQKMNSKTVTSTMLGVFREDPKTREEFLTLIRS
SEQ ID NO: 7: Tyrosine 3-hydroxylase (human)
EC = 1.14.16.2
Alternative name(s): Tyrosine 3-monooxygenase or Tyrosine 3-hydroxylase or Tyrosine
hydroxylase
Short name = TH
Organism: Homo sapiens (Human)
MPTPDATTPQAKGFRRAVSELDAKQAEAIMSPRFIGRRQSLIEDARKEREAAVAAAAAAVPSEPGDPLEAVAFEEKEGKAVLNLLFSPRA
TKPSALSRAVKVFETFEAKIHHLETRPAQRPRAGGPHLEYFVRLEVRRGDLAALLSGVRQVSEDVRSPAGPKVPWFPRKVSELDKCHHLV
TKFDPDLDLDHPGFSDQVYRQRRKLIAEIAFQYRHGDPIPRVEYTAEEIATWKEVYTTLKGLYATHACGEHLEAFALLERFSGYREDNIP
QLEDVSRFLKERTGFQLRPVAGLLSARDFLASLAFRVFQCTQYIRHASSPMHSPEPDCCHELLGHVPMLADRTFAQFSQDIGLASLGASD
EEIEKLSTLYWFTVEFGLCKQNGEVKAYGAGLLSSYGELLHCLSEEPEIRAFDPEAAAVQPYQDQTYQSVYFVSESFSDAKDKLRSYASR
IQRPFSVKFDPYTLAIDVLDSPQAVRRSLEGVQDELDTLAHALSAI
SEQ ID NO: 8: Tyrosine 3-monooxygenase (human)
>sp|P07101|TY3H_HUMAN Tyrosine 3-monooxygenase OS = Homo sapiens GN = TH PE = 1 SV = 5
MPTPDATTPQAKGFRRAVSELDAKQAEAIMVRGQGAPGPSLTGSPWPGTAAPAASYTPTPRSPRFIGRRQSLIEDARKEREAAVAAAAAA
VPSEPGDPLEAVAFEEKEGKAVLNLLFSPRATKPSALSRAVKVFETFEAKIHHLETRPAQRPRAGGPHLEYFVRLEVRRGDLAALLSGVR
QVSEDVRSPAGPKVPWFPRKVSELDKCHHLVTKFDPDLDLDHPGFSDQVYRQRRKLIAEIAFQYRHGDPIPRVEYTAEEIATWKEVYTTL
KGLYATHACGEHLEAFALLERFSGYREDNIPQLEDVSRFLKERTGFQLRPVAGLLSARDFLASLAFRVFQCTQYIRHASSPMHSPEPDCC
HELLGHVPMLADRTFAQFSQDIGLASLGASDEEIEKLSTLYWFTVEFGLCKQNGEVKAYGAGLLSSYGELLHCLSEEPEIRAFDPEAAAV
QPYQDQTYQSVYFVSESFSDAKDKLRSYASRIQRPFSVKFDPYTLAIDVLDSPQAVRRSLEGVQDELDTLAHALSAIG
SEQ ID NO: 9: Tyrosine hydroxylase (human)
>tr|Q2M3B4|Q2M3B4_HUMAN Tyrosine hydroxylase OS = Homo sapiens GN = TH PE = 2 SV = 1
MPTPDATTPQAKGFRRAVSELDAKQAEAIMSPRFIGRRQSLIEDARKEREAAVAAAAAAVPSEPGDPLEAVAFEEKEGKAMLNLLFSPRA
TKPSALSRAVKVFETFEAKIHHLETRPAQRPRAGGPHLEYFVRLEVRRGDLAALLSGVRQVSEDVRSPAGPKVPWFPRKVSELDKCHHLV
TKFDPDLDLDHPGFSDQVYRQRRKLIAEIAFQYRHGDPIPRVEYTAEEIATWKEVYTTLKGLYATHACGEHLEAFALLERFSGYREDNIP
QLEDVSRFLKERTGFQLRPVAGLLSARDFLASLAFRVFQCTQYIRHASSPMHSPEPDCCHELLGHVPMLADRTFAQFSQDIGLASLGASD
EEIEKLSTLYWFTVEFGLCKQNGEVKAYGAGLLSSYGELLHCLSEEPEIRAFDPEAAAVQPYQDQTYQSVYFVSESFSDAKDKLRSYASR
IQRPFSVKFDPYTLAIDVLDSPQAVRRSLEGVQDELDTLAHALSAIG
SEQ ID NO: 10: Tyrosine hydroxylase (human)
>tr|B7ZL73|B7ZL73_HUMAN TH protein OS = Homo sapiens GN = TH PE = 2 SV = 1
MPTPDATTPQAKGFRRAVSELDAKQAEAIMVRGQSPRFIGRRQSLIEDARKEREAAVAAAAAAVPSEPGDPLEAVAFEEKEGKAMLNLLF
SPRATKPSALSRAVKVFETFEAKIHHLETRPAQRPRAGGPHLEYFVRLEVRRGDLAALLSGVRQVSEDVRSPAGPKVPWFPRKVSELDKC
HHLVTKFDPDLDLDHPGFSDQVYRQRRKLIAEIAFQYRHGDPIPRVEYTAEEIATWKEVYTTLKGLYATHACGEHLEAFALLERFSGYRE
DNIPQLEDVSRFLKERTGFQLRPVAGLLSARDFLASLAFRVFQCTQYIRHASSPMHSPEPDCCHELLGHVPMLADHTFAQFSQDIGLASL
GASDEEIEKLSTLYWFTVEFGLCKQNGEVKAYGAGLLSSYGELLHCLSEEPEIRAFDPEAAAVQPYQDQTYQSVYFVSESFSDAKDKLRS
YASRIQRPFSVKFDPYTLAIDVLDSPQAVRRSLEGVQDELDTLAHALSAIG
SEQ ID NO: 11: Tyrosine 3-monooxygenase (human)
>sp|P07101|TY3H_HUMAN Tyrosine 3-monooxygenase OS = Homo sapiens GN = TH PE = 1 SV = 5
MPTPDATTPQAKGFRRAVSELDAKQAEAIMVRGQGAPGPSLTGSPWPGTAAPAASYTPTPRSPRFIGRRQSLIEDARKEREAAVAAAAAA
VPSEPGDPLEAVAFEEKEGKAVLNLLFSPRATKPSALSRAVKVFETFEAKIHHLETRPAQRPRAGGPHLEYFVRLEVRRGDLAALLSGVR
QVSEDVRSPAGPKVPWFPRKVSELDKCHHLVTKFDPDLDLDHPGFSDQVYRQRRKLIAEIAFQYRHGDPIPRVEYTAEEIATWKEVYTTL
KGLYATHACGEHLEAFALLERFSGYREDNIPQLEDVSRFLKERTGFQLRPVAGLLSARDFLASLAFRVFQCTQYIRHASSPMHSPEPDCC
HELLGHVPMLADRTFAQFSQDIGLASLGASDEEIEKLSTLYWFTVEFGLCKQNGEVKAYGAGLLSSYGELLHCLSEEPEIRAFDPEAAAV
QPYQDQTYQSVYFVSESFSDAKDKLRSYASRIQRPFSVKFDPYTLAIDVLDSPQAVRRSLEGVQDELDTLAHALSAIG
SEQ ID NO: 12: Truncated TH (corresponding to Catalytic domain)
MPKVPWFPRKVSELDKCHHLVTKFDPDLDLDHPGFSDQVYRQRRKLIAEIAFQYRHGDPIPRVEYTAEEIATWKEVYTTLKGLYATHACG
EHLEAFALLERFSGYREDNIPQLEDVSRFLKERTGFQLRPVAGLLSARDFLASLAFRVFQCTQYIRHASSPMHSPEPDCCHELLGHVPML
ADRTFAQFSQDIGLASLGASDEEIEKLSTLYWFTVEFGLCKQNGEVKAYGAGLLSSYGELLHCLSEEPEIRAFDPEAAAVQPYQDQTYQ
SVYFVSESFSDAKDKLRSYASRIQRPFSVKFDPYTLAIDVLDSPQAVRRSLEGVQDELDTLAHALSAIG
SEQ ID NO: 13: TH mutated at ser40
MPTPDATTPQAKGFRRAVSELDAKQAEAIMSPRFIGRRQELIEDARKEREAAVAAAAAAVPSEPGDPLEAVAFEEKEGKAVLNLLFSPRA
TKPSALSRAVKVFETFEAKIHHLETRPAQRPRAGGPHLEYFVRLEVRRGDLAALLSGVRQVSEDVRSPAGPKVPWFPRKVSELDKCHHLV
TKFDPDLDLDHPGFSDQVYRQRRKLIAEIAFQYRHGDPIPRVEYTAEEIATWKEVYTTLKGLYATHACGEHLEAFALLERFSGYREDNIP
QLEDVSRFLKERTGFQLRPVAGLLSARDFLASLAFRVFQCTQYIRHASSPMHSPEPDCCHELLGHVPMLADRTFAQFSQDIGLASLGASD
EEIEKLSTLYWFTVEFGLCKQNGEVKAYGAGLLSSYGELLHCLSEEPEIRAFDPEAAAVQPYQDQTYQSVYFVSESFSDAKDKLRSYASR
IQRPFSVKFDPYTLAIDVLDSPQAVRRSLEGVQDELDTLAHALSAIG
SEQ ID NO: 14: TH mutated at ser19 + ser40
MPTPDATTPQAKGFRRAVEELDAKQAEAIMSPRFIGRRQELIEDARKEREAAVAAAAAAVPSEPGDPLEAVAFEEKEGKAVLNLLFSPRA
TKPSALSRAVKVFETFEAKIHHLETRPAQRPRAGGPHLEYFVRLEVRRGDLAALLSGVRQVSEDVRSPAGPKVPWFPRKVSELDKCHHLV
TKFDPDLDLDHPGFSDQVYRQRRKLIAEIAFQYRHGDPIPRVEYTAEEIATWKEVYTTLKGLYATHACGEHLEAFALLERFSGYREDNIP
QLEDVSRFLKERTGFQLRPVAGLLSARDFLASLAFRVFQCTQYIRHASSPMHSPEPDCCHELLGHVPMLADRTFAQFSQDIGLASLGASD
EEIEKLSTLYWFTVEFGLCKQNGEVKAYGAGLLSSYGELLHCLSEEPEIRAFDPEAAAVQPYQDQTYQSVYFVSESFSDAKDKLRSYASR
IQRPFSVKFDPYTLAIDVLDSPQAVRRSLEGVQDELDTLAHALSAIG
SEQ ID NO: 15: TH mutated at ser19 + ser31 + ser40
MPTPDATTPQAKGFRRAVEELDAKQAEAIMEPRFIGRRQELIEDARKEREAAVAAAAAAVPSEPGDPLEAVAFEEKEGKAVLNLLFSPRA
TKPSALSRAVKVFETFEAKIHHLETRPAQRPRAGGPHLEYFVRLEVRRGDLAALLSGVRQVSEDVRSPAGPKVPWFPRKVSELDKCHHLV
TKFDPDLDLDHPGFSDQVYRQRRKLIAEIAFQYRHGDPIPRVEYTAEEIATWKEVYTTLKGLYATHACGEHLEAFALLERFSGYREDNIP
QLEDVSRFLKERTGFQLRPVAGLLSARDFLASLAFRVFQCTQYIRHASSPMHSPEPDCCHELLGHVPMLADRTFAQFSQDIGLASLGASD
EEIEKLSTLYWFTVEFGLCKQNGEVKAYGAGLLSSYGELLHCLSEEPEIRAFDPEAAAVQPYQDQTYQSVYFVSESFSDAKDKLRSYASR
IQRPFSVKFDPYTLAIDVLDSPQAVRRSLEGVQDELDTLAHALSAIG
SEQ ID NO: 16: Tyrosine 3-hydroxylase (rat)
>sp|P04177|TY3H_RAT Tyrosine 3-monooxygenase OS = Rattus norvegicus GN = Th PE = 1 SV = 3
MPTPSAPSPQPKGFRRAVSEQDAKQAEAVTSPRFIGRRQSLIEDARKEREAAAAAAAAAVASSEPGNPLEAVVFEERDGNAVLNLLFSLR
GTKPSSLSRAVKVFETFEAKIHHLETRPAQRPLAGSPHLEYFVRFEVPSGDLAALLSSVRRVSDDVRSAREDKVPWFPRKVSELDKCHHL
VTKFDPDLDLDHPGFSDQVYRQRRKLIAEIAFQYKHGEPIPHVEYTAEEIATWKEVYVTLKGLYATHACREHLEGFOLLERYCGYREDSI
PQLEDVSRFLKERTGFQLRPVAGLLSARDFLASLAFRVFQCTQYIRHASSPMHSPEPDCCHELLGHVPMLADRTFAQFSQDIGLASLGAS
DEEIEKLSTVYWFTVEFGLCKQNGELKAYGAGLLSSYGELLHSLSEEPEVRAFDPDTAAVQPYQDQTYQPVYFVSESFNDAKDKLRNYAS
RIQRPFSVKFDPYTLAIDVLDSPHTIQRSLEGVQDELHTLAHALSAIS
SEQ ID NO: 17: Tyrosine 3-hydroxylase (mouse)
>sp|P24529|TY3H_MOUSE Tyrosine 3-monooxygenase OS = Mus musculus GN = Th PE = 1 SV = 3
MPTPSASSPQPKGFRRAVSEQDTKQAEAVTSPRFIGRRQSLIEDARKEREAAAAAAAAAVASAEPGNPLEAVVFEERDGNAVLNLLFSLR
GTKPSSLSRALKVFETFEAKIHHLETRPAQRPLAGSPHLEYFVRFEVPSGDLAALLSSVRRVSDDVRSAREDKVPWFPRKVSELDKCHHL
VTKFDPDLDLDHPGFSDQAYRQRRKLIAEIAFQYKQGEPIPHVEYTKEEIATWKEVYATLKGLYATHACREHLEAFQLLERYCGYREDSI
PQLEDVSHFLKERTGFQLRPVAGLLSARDFLASLAFRVFQCTQYIRHASSPMHSPEPDCCHELLGHVPMLADRTFAQFSQDIGLASLGAS
DEEIEKLSTVYWFTVEFGLCKQNGELKAYGAGLLSSYGELLHSLSEEPEVRAFDPDTAAVQPYQDQTYQPVYFVSESFSDAKDKLRNYAS
RIQRPFSVKFDPYTLAIDVLDSPHTIRRSLEGVQDELHTLTQALSAIS
SEQ ID NO: 18: Adeno-associated virus 2 left terminal nucleotide sequence
ttggccactccctctctgcgcgctcgctcgctcactgaggccgggcgaccaaaggtcgcccgacgcccgggctttgcccgggcggcctca
gtgagcgagcgagcgcgcagagagggagtggccaactccatcactaggggttcct
SEQ ID NO: 19: Adeno-associated virus 2 right terminal nucleotide sequence
aggaacccctagtgatggagttggccactccctctctgcgcgctcgctcgctcactgaggccgcccgggcaaagcccgggcgtcgggcga
cctttggtcgcccggcctcagtgagcgagcgagcgcgcagagagggagtggccaa
SEQ ID NO: 20: Homo sapiens GTP cyclohydrolase 1 (GCH1), transcript variant 1
ATGGAGAAGGGCCCTGTGCGGGCACCGGCGGAGAAGCCGCGGGGCGCCAGGTGCAGCAATGGGTTCCCCGAGCGGGATCCGCCGCGGCCC
GGGCCCAGCAGGCCGGCGGAGAAGCCCCCGCGGCCCGAGGCCAAGAGCGCGCAGCCCGCGGACGGCTGGAAGGGCGAGCGGCCCCGCAGC
GAGGAGGATAACGAGCTGAACCTCCCTAACCTGGCAGCCGCCTACTCGTCCATCCTGAGCTCGCTGGGCGAGAACCCCCAGCGGCAAGGG
CTGCTCAAGACGCCCTGGAGGGCGGCCTCGGCCATGCAGTTCTTCACCAAGGGCTACCAGGAGACCATCTCAGATGTCCTAAACGATGCT
ATATTTGATGAAGATCATGATGAGATGGTGATTGTGAAGGACATAGACATGTTTTCCATGTGTGAGCATCACTTGGTTCCATTTGTTGGA
AAGGTCCATATTGGTTATCTTCCTAACAAGCAAGTCCTTGGCCTCAGCAAACTTGCGAGGATTGTAGAAATCTATAGTAGAAGACTACAA
GTTCAGGAGCGCCTTACAAAACAAATTGCTGTAGCAATCACGGAAGCCTTGCGGCCTGCTGGAGTCGGGGTAGTGGTTGAAGCAACACAC
ATGTGTATGGTAATGCGAGGTGTACAGAAAATGAACAGCAAAACTGTGACCAGCACAATGTTGGGTGTGTTCCGGGAGGATCCAAAGACT
CGGGAAGAGTTCCTGACTCTCATTAGGAGCTAA
SEQ ID NO: 21: Simian virus 40 early poly-adenylation nucleotide sequence
TTCGAGCAACTTGTTTATTGCAGCTTATAATGGTTACAAATAAAGCAATAGCATCACAAATTTCACAAATAAAGCATTTTTTTCACTGCA
TTCTAGTTGTGGTTTGTCCAAACTCATCAATGTATCTTATCATGTCTGGATCGTCTAGCATCGAA
SEQ ID NO: 22: Simian virus 40 late poly-adenylation nucleotide sequence
CAGACATGATAAGATACATTGATGAGTTTGGACAAACCACAACTAGAATGCAGTGAAAAAAATGCTTTATTTGTGAAATTTGTGATGCTA
TTGCTTTATTTGTAACCATTATAAGCTGCAATAAACAAGTTAACAACAACAATTGCATTCATTTTATGTTTCAGGTTCAGGGGGAGGTGT
GGGAGGTTTTTT
SEQ ID NO: 23: Homo sapiens tyrosine hydroxylase (TH), transcript variant 2 nucleotide
sequence
ATGCCCACCCCCGACGCCACCACGCCACAGGCCAAGGGCTTCCGCAGGGCCGTGTCTGAGCTGGACGCCAAGCAGGCAGAGGCCATCATG
TCCCCGCGGTTCATTGGGCGCAGGCAGAGCCTCATCGAGGACGCCCGCAAGGAGCGGGAGGCGGCGGTGGCAGCAGCGGCCGCTGCAGTC
CCCTCGGAGCCCGGGGACCCCCTGGAGGCTGTGGCCTTTGAGGAGAAGGAGGGGAAGGCCGTGCTAAACCTGCTCTTCTCCCCGAGGGCC
ACCAAGCCCTCGGCGCTGTCCCGAGCTGTGAAGGTGTTTGAGACGTTTGAAGCCAAAATCCACCATCTAGAGACCCGGCCCGCCCAGAGG
CCGCGAGCTGGGGGCCCCCACCTGGAGTACTTCGTGCGCCTCGAGGTGCGCCGAGGGGACCTGGCCGCCCTGCTCAGTGGTGTGCGCCAG
GTGTCAGAGGACGTGCGCAGCCCCGCGGGGCCCAAGGTCCCCTGGTTCCCAAGAAAAGTGTCAGAGCTGGACAAGTGTCATCACCTGGTC
ACCAAGTTCGACCCTGACCTGGACTTGGACCACCCGGGCTTCTCGGACCAGGTGTACCGCCAGCGCAGGAAGCTGATTGCTGAGATCGCC
TTCCAGTACAGGCACGGCGACCCGATTCCCCGTGTGGAGTACACCGCCGAGGAGATTGCCACCTGGAAGGAGGTCTACACCACGCTGAAG
GGCCTCTACGCCACGCACGCCTGCGGGGAGCACCTGGAGGCCTTTGCTTTGCTGGAGCGCTTCAGCGGCTACCGGGAAGACAATATCCCC
CAGCTGGAGGACGTCTCCCGCTTCCTGAAGGAGCGCACGGGCTTCCAGCTGCGGCCTGTGGCCGGCCTGCTGTCCGCCCGGGACTTCCTG
GCCAGCCTGGCCTTCCGCGTGTTCCAGTGCACCCAGTATATCCGCCACGCGTCCTCGCCCATGCACTCCCCTGAGCCGGACTGCTGCCAC
GAGCTGCTGGGGCACGTGCCCATGCTGGCCGACCGCACCTTCGCGCAGTTCTCGCAGGACATTGGCCTGGCGTCCCTGGGGGCCTCGGAT
GAGGAAATTGAGAAGCTGTCCACGCTGTACTGGTTCACGGTGGAGTTCGGGCTGTGTAAGCAGAACGGGGAGGTGAAGGCCTATGGTGCC
GGGCTGCTGTCCTCCTACGGGGAGCTCCTGCACTGCCTGTCTGAGGAGCCTGAGATTCGGGCCTTCGACCCTGAGGCTGCGGCCGTGCAG
CCCTACCAAGACCAGACGTACCAGTCAGTCTACTTCGTGTCTGAGAGCTTCAGTGACGCCAAGGACAAGCTCAGGAGCTATGCCTCACGC
ATCCAGCGCCCCTTCTCCGTGAAGTTCGACCCGTACACGCTGGCCATCGACGTGCTGGACAGCCCCCAGGCCGTGCGGCGCTCCCTGGAG
GGTGTCCAGGATGAGCTGGACACCCTTGCCCATGCGCTGAGTGCCATTGG
SEQ ID NO: 24: Truncated TH (encoding catalytic domain), nucleotide sequence
ATGAGCCCCGCGGGGCCCAAGGTCCCCTGGTTCCCAAGAAAAGTGTCAGAGCTGGACAAGTGTCATCACCTGGTCACCAAGTTCGACCCT
GACCTGGACTTGGACCACCCGGGCTTCTCGGACCAGGTGTACCGCCAGCGCAGGAAGCTGATTGCTGAGATCGCCTTCCAGTACAGGCAC
GGCGACCCGATTCCCCGTGTGGAGTACACCGCCGAGGAGATTGCCACCTGGAAGGAGGTCTACACCACGCTGAAGGGCCTCTACGCCACG
CACGCCTGCGGGGAGCACCTGGAGGCCTTTGCTTTGCTGGAGCGCTTCAGCGGCTACCGGGAAGACAATATCCCCCAGCTGGAGGACGTC
TCCCGCTTCCTGAAGGAGCGCACGGGCTTCCAGCTGCGGCCTGTGGCCGGCCTGCTGTCCGCCCGGGACTTCCTGGCCAGCCTGGCCTTC
CGCGTGTTCCAGTGCACCCAGTATATCCGCCACGCGTCCTCGCCCATGCACTCCCCTGAGCCGGACTGCTGCCACGAGCTGCTGGGGCAC
GTGCCCATGCTGGCCGACCGCACCTTCGCGCAGTTCTCGCAGGACATTGGCCTGGCGTCCCTGGGGGCCTCGGATGAGGAAATTGAGAAG
CTGTCCACGCTGTACTGGTTCACGGTGGAGTTCGGGCTGTGTAAGCAGAACGGGGAGGTGAAGGCCTATGGTGCCGGGCTGCTGTCCTCC
TACGGGGAGCTCCTGCACTGCCTGTCTGAGGAGCCTGAGATTCGGGCCTTCGACCCTGAGGCTGCGGCCGTGCAGCCCTACCAAGACCAG
ACGTACCAGTCAGTCTACTTCGTGTCTGAGAGCTTCAGTGACGCCAAGGACAAGCTCAGGAGCTATGCCTCACGCATCCAGCGCCCCTTC
TCCGTGAAGTTCGACCCGTACACGCTGGCCATCGACGTGCTGGACAGCCCCCAGGCCGTGCGGCGCTCCCTGGAGGGTGTCCAGGATGAG
CTGGACACCCTTGCCCATGCGCTGAGTGCCATTGGCTAA
SEQ ID NO: 25: TH mutated at ser40, nucleotide sequence
ATGCCCACCCCCGACGCCACCACGCCACAGGCCAAGGGCTTCCGCAGGGCCGTGTCTGAGCTGGACGCCAAGCAGGCAGAGGCCATCATG
TCCCCGCGGTTCATTGGGCGCAGGCAGGAGCTCATCGAGGACGCCCGCAAGGAGCGGGAGGCGGCGGTGGCAGCAGCGGCCGCTGCAGTC
CCCTCGGAGCCCGGGGACCCCCTGGAGGCTGTGGCCTTTGAGGAGAAGGAGGGGAAGGCCGTGCTAAACCTGCTCTTCTCCCCGAGGGCC
ACCAAGCCCTCGGCGCTGTCCCGAGCTGTGAAGGTGTTTGAGACGTTTGAAGCCAAAATCCACCATCTAGAGACCCGGCCCGCCCAGAGG
CCGCGAGCTGGGGGCCCCCACCTGGAGTACTTCGTGCGCCTCGAGGTGCGCCGAGGGGACCTGGCCGCCCTGCTCAGTGGTGTGCGCCAG
GTGTCAGAGGACGTGCGCAGCCCCGCGGGGCCCAAGGTCCCCTGGTTCCCAAGAAAAGTGTCAGAGCTGGACAAGTGTCATCACCTGGTC
ACCAAGTTCGACCCTGACCTGGACTTGGACCACCCGGGCTTCTCGGACCAGGTGTACCGCCAGCGCAGGAAGCTGATTGCTGAGATCGCC
TTCCAGTACAGGCACGGCGACCCGATTCCCCGTGTGGAGTACACCGCCGAGGAGATTGCCACCTGGAAGGAGGTCTACACCACGCTGAAG
GGCCTCTACGCCACGCACGCCTGCGGGGAGCACCTGGAGGCCTTTGCTTTGCTGGAGCGCTTCAGCGGCTACCGGGAAGACAATATCCCC
CAGCTGGAGGACGTCTCCCGCTTCCTGAAGGAGCGCACGGGCTTCCAGCTGCGGCCTGTGGCCGGCCTGCTGTCCGCCCGGGACTTCCTG
GCCAGCCTGGCCTTCCGCGTGTTCCAGTGCACCCAGTATATCCGCCACGCGTCCTCGCCCATGCACTCCCCTGAGCCGGACTGCTGCCAC
GAGCTGCTGGGGCACGTGCCCATGCTGGCCGACCGCACCTTCGCGCAGTTCTCGCAGGACATTGGCCTGGCGTCCCTGGGGGCCTCGGAT
GAGGAAATTGAGAAGCTGTCCACGCTGTACTGGTTCACGGTGGAGTTCGGGCTGTGTAAGCAGAACGGGGAGGTGAAGGCCTATGGTGCC
GGGCTGCTGTCCTCCTACGGGGAGCTCCTGCACTGCCTGTCTGAGGAGCCTGAGATTCGGGCCTTCGACCCTGAGGCTGCGGCCGTGCAG
CCCTACCAAGACCAGACGTACCAGTCAGTCTACTTCGTGTCTGAGAGCTTCAGTGACGCCAAGGACAAGCTCAGGAGCTATGCCTCACGC
ATCCAGCGCCCCTTCTCCGTGAAGTTCGACCCGTACACGCTGGCCATCGACGTGCTGGACAGCCCCCAGGCCGTGCGGCGCTCCCTGGAG
GGTGTCCAGGATGAGCTGGACACCCTTGCCCATGCGCTGAGTGCCATTGGC
SEQ ID NO: 26: TH mutated as ser19 and ser40, nucleotide sequence
ATGCCCACCCCCGACGCCACCACGCCACAGGCCAAGGGCTTCCGCAGGGCCGTGGAGGAGCTGGACGCCAAGCAGGCAGAGGCCATCATG
TCCCCGCGGTTCATTGGGCGCAGGCAGGAGCTCATCGAGGACGCCCGCAAGGAGCGGGAGGCGGCGGTGGCAGCAGCGGCCGCTGCAGTC
CCCTCGGAGCCCGGGGACCCCCTGGAGGCTGTGGCCTTTGAGGAGAAGGAGGGGAAGGCCGTGCTAAACCTGCTCTTCTCCCCGAGGGCC
ACCAAGCCCTCGGCGCTGTCCCGAGCTGTGAAGGTGTTTGAGACGTTTGAAGCCAAAATCCACCATCTAGAGACCCGGCCCGCCCAGAGG
CCGCGAGCTGGGGGCCCCCACCTGGAGTACTTCGTGCGCCTCGAGGTGCGCCGAGGGGACCTGGCCGCCCTGCTCAGTGGTGTGCGCCAG
GTGTCAGAGGACGTGCGCAGCCCCGCGGGGCCCAAGGTCCCCTGGTTCCCAAGAAAAGTGTCAGAGCTGGACAAGTGTCATCACCTGGTC
ACCAAGTTCGACCCTGACCTGGACTTGGACCACCCGGGCTTCTCGGACCAGGTGTACCGCCAGCGCAGGAAGCTGATTGCTGAGATCGCC
TTCCAGTACAGGCACGGCGACCCGATTCCCCGTGTGGAGTACACCGCCGAGGAGATTGCCACCTGGAAGGAGGTCTACACCACGCTGAAG
GGCCTCTACGCCACGCACGCCTGCGGGGAGCACCTGGAGGCCTTTGCTTTGCTGGAGCGCTTCAGCGGCTACCGGGAAGACAATATCCCC
CAGCTGGAGGACGTCTCCCGCTTCCTGAAGGAGCGCACGGGCTTCCAGCTGCGGCCTGTGGCCGGCCTGCTGTCCGCCCGGGACTTCCTG
GCCAGCCTGGCCTTCCGCGTGTTCCAGTGCACCCAGTATATCCGCCACGCGTCCTCGCCCATGCACTCCCCTGAGCCGGACTGCTGCCAC
GAGCTGCTGGGGCACGTGCCCATGCTGGCCGACCGCACCTTCGCGCAGTTCTCGCAGGACATTGGCCTGGCGTCCCTGGGGGCCTCGGAT
GAGGAAATTGAGAAGCTGTCCACGCTGTACTGGTTCACGGTGGAGTTCGGGCTGTGTAAGCAGAACGGGGAGGTGAAGGCCTATGGTGCC
GGGCTGCTGTCCTCCTACGGGGAGCTCCTGCACTGCCTGTCTGAGGAGCCTGAGATTCGGGCCTTCGACCCTGAGGCTGCGGCCGTGCAG
CCCTACCAAGACCAGACGTACCAGTCAGTCTACTTCGTGTCTGAGAGCTTCAGTGACGCCAAGGACAAGCTCAGGAGCTATGCCTCACGC
ATCCAGCGCCCCTTCTCCGTGAAGTTCGACCCGTACACGCTGGCCATCGACGTGCTGGACAGCCCCCAGGCCGTGCGGCGCTCCCTGGAG
GGTGTCCAGGATGAGCTGGACACCCTTGCCCATGCGCTGAGTGCCATTGGC
SEQ ID NO: 27: TH mutated as ser19, ser31 and ser40, nucleotide sequence
ATGCCCACCCCCGACGCCACCACGCCACAGGCCAAGGGCTTCCGCAGGGCCGTGGAGGAGCTGGACGCCAAGCAGGCAGAGGCCATCATG
GAGCCGCGGTTCATTGGGCGCAGGCAGGAGCTCATCGAGGACGCCCGCAAGGAGCGGGAGGCGGCGGTGGCAGCAGCGGCCGCTGCAGTC
CCCTCGGAGCCCGGGGACCCCCTGGAGGCTGTGGCCTTTGAGGAGAAGGAGGGGAAGGCCGTGCTAAACCTGCTCTTCTCCCCGAGGGCC
ACCAAGCCCTCGGCGCTGTCCCGAGCTGTGAAGGTGTTTGAGACGTTTGAAGCCAAAATCCACCATCTAGAGACCCGGCCCGCCCAGAGG
CCGCGAGCTGGGGGCCCCCACCTGGAGTACTTCGTGCGCCTCGAGGTGCGCCGAGGGGACCTGGCCGCCCTGCTCAGTGGTGTGCGCCAG
GTGTCAGAGGACGTGCGCAGCCCCGCGGGGCCCAAGGTCCCCTGGTTCCCAAGAAAAGTGTCAGAGCTGGACAAGTGTCATCACCTGGTC
ACCAAGTTCGACCCTGACCTGGACTTGGACCACCCGGGCTTCTCGGACCAGGTGTACCGCCAGCGCAGGAAGCTGATTGCTGAGATCGCC
TTCCAGTACAGGCACGGCGACCCGATTCCCCGTGTGGAGTACACCGCCGAGGAGATTGCCACCTGGAAGGAGGTCTACACCACGCTGAAG
GGCCTCTACGCCACGCACGCCTGCGGGGAGCACCTGGAGGCCTTTGCTTTGCTGGAGCGCTTCAGCGGCTACCGGGAAGACAATATCCCC
CAGCTGGAGGACGTCTCCCGCTTCCTGAAGGAGCGCACGGGCTTCCAGCTGCGGCCTGTGGCCGGCCTGCTGTCCGCCCGGGACTTCCTG
GCCAGCCTGGCCTTCCGCGTGTTCCAGTGCACCCAGTATATCCGCCACGCGTCCTCGCCCATGCACTCCCCTGAGCCGGACTGCTGCCAC
GAGCTGCTGGGGCACGTGCCCATGCTGGCCGACCGCACCTTCGCGCAGTTCTCGCAGGACATTGGCCTGGCGTCCCTGGGGGCCTCGGAT
GAGGAAATTGAGAAGCTGTCCACGCTGTACTGGTTCACGGTGGAGTTCGGGCTGTGTAAGCAGAACGGGGAGGTGAAGGCCTATGGTGCC
GGGCTGCTGTCCTCCTACGGGGAGCTCCTGCACTGCCTGTCTGAGGAGCCTGAGATTCGGGCCTTCGACCCTGAGGCTGCGGCCGTGCAG
CCCTACCAAGACCAGACGTACCAGTCAGTCTACTTCGTGTCTGAGAGCTTCAGTGACGCCAAGGACAAGCTCAGGAGCTATGCCTCACGC
ATCCAGCGCCCCTTCTCCGTGAAGTTCGACCCGTACACGCTGGCCATCGACGTGCTGGACAGCCCCCAGGCCGTGCGGCGCTCCCTGGAG
GGTGTCCAGGATGAGCTGGACACCCTTGCCCATGCGCTGAGTGCCATTGGC
SEQ ID NO: 28: Woodchuck hepatitis B virus (WHV8) post-transcriptional regulatory element
nucleotide sequence
CGTCGACAATCAACCTCTGGATTACAAAATTTGTGAAAGATTGACTGGTATTCTTAACTATGTTGCTCCTTTTACGCTATGTGGATACGC
TGCTTTAATGCCTTTGTATCATGCTATTGCTTCCCGTATGGCTTTCATTTTCTCCTCCTTGTATAAATCCTGGTTGCTGTCTCTTTATGA
GGAGTTGTGGCCCGTTGTCAGGCAACGTGGCGTGGTGTGCACTGTGTTTGCTGACGCAACCCCCACTGGTTGGGGCATTGCCACCACCTG
TCAGCTCCTTTCCGGGACTTTCGCTTTCCCCCTCCCTATTGCCACGGCGGAACTCATCGCCGCCTGCCTTGCCCGCTGCTGGACAGGGGC
TCGGCTGTTGGGCACTGACAATTCCGTGGTGTTGTCGGGGAAGCTGACGTCCTTTCCATGGCTGCTCGCCTGTGTTGCCACCTGGATTCT
GCGCGGGACGTCCTTCTGCTACGTCCCTTCGGCCCTCAATCCAGCGGACCTTCCTTCCCGCGGCCTGCTGCCGGCTCTGCGGCCTCTTCC
GCGTCTTCGCCTTCGCCCTCAGACGAGTCGGATCTCCCTTTGGGCCGCCTCCCCGCCTGGAATTCGAGCT
SEQ ID NO: 29: Mutated Woodchuck hepatitis B virus (WHV8) post-transcriptional regulatory
element nucleotide sequence
CGTCGATAATCAACCTCTGGATTACAAAATTTGTGAAAGATTGACTGGTATTCTTAACTATGTTGCTCCTTTTACGCTATGTGGATACGC
TGCTTTAATGCCTTTGTATCATGCTATTGCTTCCCGTATGGCTTTCATTTTCTCCTCCTTGTATAAATCCTGGTTGCTGTCTCTTTATGA
GGAGTTGTGGCCCGTTGTCAGGCAACGTGGCGTGGTGTGCACTGTGTTTGCTGACGCAACCCCCACTGGTTGGGGCATTGCCACCACCTG
TCAGCTCCTTTCCGGGACTTTCGCTTTCCCCCTCCCTATTGCCACGGCGGAACTCATCGCCGCCTGCCTTGCCCGCTGCTGGACAGGGGC
TCGGCTGTTGGGCACTGACAATTCCGTGGTGTTGTCGGGGAAATCATCGTCCTTTCCTTGGCTGCTCGCCTGTGTTGCCACCTGGATTCT
GCGCGGGACGTCCTTCTGCTACGTCCCTTCGGCCCTCAATCCAGCGGACCTTCCTTCCCGCGGCCTGCTGCCGGCTCTGCGGCCTCTTCC
GCGTCTTCGCCTTCGCCCTCAGACGAGTCGGATCTCCCTTTGGGCCGCCTCCCCGCCTGGAATTCGAGCT
SEQ ID NO: 30 GCH1 nucleotide sequence
ATGGAGAAGGGCCCTGTGCGGGCACCGGCGGAGAAGCCGCGGGGCGCCAGGTGCAGCAATGGGTTCCCCGAGCGGGATCCGCCGCGGCCC
GGGCCCAGCAGGCCGGCGGAGAAGCCCCCGCGGCCCGAGGCCAAGAGCGCGCAGCCCGCGGACGGCTGGAAGGGCGAGCGGCCCCGCAGC
GAGGAGGATAACGAGCTGAACCTCCCTAACCTGGCAGCCGCCTACTCGTCCATCCTGAGCTCGCTGGGCGAGAACCCCCAGCGGCAAGGG
CTGCTCAAGACGCCCTGGAGGGCGGCCTCGGCCATGCAGTTCTTCACCAAGGGCTACCAGGAGACCATCTCAGATGTCCTAAACGATGCT
ATATTTGATGAAGATCATGATGAGATGGTGATTGTGAAGGACATAGACATGTTTTCCATGTGTGAGCATCACTTGGTTCCATTTGTTGGA
AAGGTCCATATTGGTTATCTTCCTAACAAGCAAGTCCTTGGCCTCAGCAAACTTGCGAGGATTGTAGAAATCTATAGTAGAAGACTACAA
GTTCAGGAGCGCCTTACAAAACAAATTGCTGTAGCAATCACGGAAGCCTTGCGGCCTGCTGGAGTCGGGGTAGTGGTTGAAGCAACACAC
ATGTGTATGGTAATGCGAGGTGTACAGAAAATGAACAGCAAAACTGTGACCAGCACAATGTTGGGTGTGTTCCGGGAGGATCCAAAGACT
CGGGAAGAGTTCCTGACTCTCATTAGGA
SEQ ID NO: 31 pAA011-scAAV-HLP-GCH1:
AAAGCTTCCCGGGGGGATCTGGGCCACTCCCTCTCTGCGCGCTCGCTCGCTCACTGAGGCCGGGCGACCAAAGGTCGCCCGACGCCCGGG
CTTTGCCCGGGCGGCCTCAGTGAGCGAGCGAGCGCGCAGAGAGGGAGTGGCCAACTCCATCACTAGGGGTTCCTTCTAGATGTTTGCTGC
TTGCAATGTTTGCCCATTTTAGGGTGGACACAGGACGCTGTGGTTTCTGAGCCAGGGGGCGACTCAGATCCCAGCCAGTGGACTTAGCCC
CTGTTTGCTCCTCCGATAACTGGGGTGACCTTGGTTAATATTCACCAGCAGCCTCCCCCGTTGCCCCTCTGGATCCACTGCTTAAATACG
GACGAGGACAGGGCCCTGTCTCCTCAGCTTCAGGCACCACCACTGACCTGGGACAGTGAATCGCTAGCGAATTCTAGCATGGAGAAGGGC
CCTGTGCGGGCACCGGCGGAGAAGCCGCGGGGCGCCAGGTGCAGCAATGGGTTCCCCGAGCGGGATCCGCCGCGGCCCGGGCCCAGCAGG
CCGGCGGAGAAGCCCCCGCGGCCCGAGGCCAAGAGCGCGCAGCCCGCGGACGGCTGGAAGGGCGAGCGGCCCCGCAGCGAGGAGGATAAC
GAGCTGAACCTCCCTAACCTGGCAGCCGCCTACTCGTCCATCCTGAGCTCGCTGGGCGAGAACCCCCAGCGGCAAGGGCTGCTCAAGACG
CCCTGGAGGGCGGCCTCGGCCATGCAGTTCTTCACCAAGGGCTACCAGGAGACCATCTCAGATGTCCTAAACGATGCTATATTTGATGAA
GATCATGATGAGATGGTGATTGTGAAGGACATAGACATGTTTTCCATGTGTGAGCATCACTTGGTTCCATTTGTTGGAAAGGTCCATATT
GGTTATCTTCCTAACAAGCAAGTCCTTGGCCTCAGCAAACTTGCGAGGATTGTAGAAATCTATAGTAGAAGACTACAAGTTCAGGAGCGC
CTTACAAAACAAATTGCTGTAGCAATCACGGAAGCCTTGCGGCCTGCTGGAGTCGGGGTAGTGGTTGAAGCAACACACATGTGTATGGTA
ATGCGAGGTGTACAGAAAATGAACAGCAAAACTGTGACCAGCACAATGTTGGGTGTGTTCCGGGAGGATCCAAAGACTCGGGAAGAGTTC
CTGACTCTCATTAGGAGCTAATGCATCCCCATCGATGATCCAGACATGATAAGATACATTGATGAGTTTGGACAAACCACAACTAGAATG
CAGTGAAAAAAATGCTTTATTTGTGAAATTTGTGATGCTATTGCTTTATTTGTAACCATTATAAGCTGCAATAAACAAGTTAACAACAAC
AATTGCATTCATTTTATGTTTCAGGTTCAGGGGGAGGTGTGGGAGGTTTTTTAGTCGACCGCTAGTCCACTCCCTCTCTGCGCGCTCGCT
CGCTCACTGAGGCCGGGCGACCAAAGGTCGCCCGACGCCCGGGCTTTGCCCGGGCGGCCTCAGTGAGCGAGCGAGCGCGCAGAGAGGGAC
AGATCCGGGCCCGCATGCGTCGACAATTCACTGGCCGTCGTTTTACAACGTCGTGACTGGGAAAACCCTGGCGTTACCCAACTTAATCGC
CTTGCAGCACATCCCCCTTTCGCCAGCTGGCGTAATAGCGAAGAGGCCCGCACCGATCGCCCTTCCCAACAGTTGCGCAGCCTGAATGGC
GAATGGCGCCTGATGCGGTATTTTCTCCTTACGCATCTGTGCGGTATTTCACACCGCATATGGTGCACTCTCAGTACAATCTGCTCTGAT
GCCGCATAGTTAAGCCAGCCCCGACACCCGCCAACACCCGCTGACGCGCCCTGACGGGCTTGTCTGCTCCCGGCATCCGCTTACAGACAA
GCTGTGACCGTCTCCGGGAGCTGCATGTGTCAGAGGTTTTCACCGTCATCACCGAAACGCGCGAGACGAAAGGGCCTCGTGATACGCCTA
TTTTTATAGGTTAATGTCATGATAATAATGGTTTCTTAGACGTCAGGTGGCACTTTTCGGGGAAATGTGCGCGGAACCCCTATTTGTTTA
TTTTTCTAAATACATTCAAATATGTATCCGCTCATGAGACAATAACCCTGATAAATGCTTCAATAATATTGAAAAAGGAAGAGTATGAGT
ATTCAACATTTCCGTGTCGCCCTTATTCCCTTTTTTGCGGCATTTTGCCTTCCTGTTTTTGCTCACCCAGAAACGCTGGTGAAAGTAAAA
GATGCTGAAGATCAGTTGGGTGCACGAGTGGGTTACATCGAACTGGATCTCAACAGCGGTAAGATCCTTGAGAGTTTTCGCCCCGAAGAA
CGTTTTCCAATGATGAGCACTTTTAAAGTTCTGCTATGTGGCGCGGTATTATCCCGTATTGACGCCGGGCAAGAGCAACTCGGTCGCCGC
ATACACTATTCTCAGAATGACTTGGTTGAGTACTCACCAGTCACAGAAAAGCATCTTACGGATGGCATGACAGTAAGAGAATTATGCAGT
GCTGCCATAACCATGAGTGATAACACTGCGGCCAACTTACTTCTGACAACGATCGGAGGACCGAAGGAGCTAACCGCTTTTTTGCACAAC
ATGGGGGATCATGTAACTCGCCTTGATCGTTGGGAACCGGAGCTGAATGAAGCCATACCAAACGACGAGCGTGACACCACGATGCCTGTA
GCAATGGCAACAACGTTGCGCAAACTATTAACTGGCGAACTACTTACTCTAGCTTCCCGGCAACAATTAATAGACTGGATGGAGGCGGAT
AAAGTTGCAGGACCACTTCTGCGCTCGGCCCTTCCGGCTGGCTGGTTTATTGCTGATAAATCTGGAGCCGGTGAGCGTGGGTCTCGCGGT
ATCATTGCAGCACTGGGGCCAGATGGTAAGCCCTCCCGTATCGTAGTTATCTACACGACGGGGAGTCAGGCAACTATGGATGAACGAAAT
AGACAGATCGCTGAGATAGGTGCCTCACTGATTAAGCATTGGTAACTGTCAGACCAAGTTTACTCATATATACTTTAGATTGATTTAAAA
CTTCATTTTTAATTTAAAAGGATCTAGGTGAAGATCCTTTTTGATAATCTCATGACCAAAATCCCTTAACGTGAGTTTTCGTTCCACTGA
GCGTCAGACCCCGTAGAAAAGATCAAAGGATCTTCTTGAGATCCTTTTTTTCTGCGCGTAATCTGCTGCTTGCAAACAAAAAAACCACCG
CTACCAGCGGTGGTTTGTTTGCCGGATCAAGAGCTACCAACTCTTTTTCCGAAGGTAACTGGCTTCAGCAGAGCGCAGATACCAAATACT
GTTCTTCTAGTGTAGCCGTAGTTAGGCCACCACTTCAAGAACTCTGTAGCACCGCCTACATACCTCGCTCTGCTAATCCTGTTACCAGTG
GCTGCTGCCAGTGGCGATAAGTCGTGTCTTACCGGGTTGGACTCAAGACGATAGTTACCGGATAAGGCGCAGCGGTCGGGCTGAACGGGG
GGTTCGTGCACACAGCCCAGCTTGGAGCGAACGACCTACACCGAACTGAGATACCTACAGCGTGAGCTATGAGAAAGCGCCACGCTTCCC
GAAGGGAGAAAGGCGGACAGGTATCCGGTAAGCGGCAGGGTCGGAACAGGAGAGCGCACGAGGGAGCTTCCAGGGGGAAACGCCTGGTAT
CTTTATAGTCCTGTCGGGTTTCGCCACCTCTGACTTGAGCGTCGATTTTTGTGATGCTCGTCAGGGGGGCGGAGCCTATGGAAAAACGCC
AGCAACGCGGCCTTTTTACGGTTCCTGGCCTTTTGCTGGCCTTTTGCTCACATGTTCTTTCCTGCGTTATCCCCTGATTCTGTGGATAAC
CGTATTACCGCCTTTGAGTGAGCTGATACCGCTCGCCGCAGCCGAACGACCGAGCGCAGCGAGTCAGTGAGCGAGGAAGCGGAAGAGCGC
CCAATACGCAAACCGCCTCTCCCCGCGCGTTGGCCGATTCATTAATGCAGCTGGCACGACAGGTTTCCCGACTGGAAAGCGGGCAGTGAG
CGCAACGCAATTAATGTGAGTTAGCTCACTCATTAGGCACCCCAGGCTTTACACTTTATGCTTCCGGCTCGTATGTTGTGTGGAATTGTG
AGCGGATAACAATTTCACACAGGAAACAGCTATGACCATGATTACGCCAAGCTCTCGAGATCTAG
SEQ ID NO: 32: pAA016-scAAV-HLP-tTH:
AAAGCTTCCCGGGGGGATCTGGGCCACTCCCTCTCTGCGCGCTCGCTCGCTCACTGAGGCCGGGCGACCAAAGGTCGCCCGACGCCCGGG
CTTTGCCCGGGCGGCCTCAGTGAGCGAGCGAGCGCGCAGAGAGGGAGTGGCCAACTCCATCACTAGGGGTTCCTGGAGGGGTGGAGTCGT
GACCCCTAAAATGGGCAAACATTGCGCTAGCTGTTTGCTGCTTGCAATGTTTGCCCATTTTAGGGTGGACACAGGACGCTGTGGTTTCTG
AGCCAGGGGGCGACTCAGATCCCAGCCAGTGGACTTAGCCCCTGTTTGCTCCTCCGATAACTGGGGTGACCTTGGTTAATATTCACCAGC
AGCCTCCCCCGTTGCCCCTCTGGATCCACTGCTTAAATACGGACGAGGACAGGGCCCTGTCTCCTCAGCTTCAGGCACCACCACTGACCT
GGGACAGTGAATCATTCAAGCTCGTAGCAAGGATCCACCGGTCACCATGAGCCCCGCGGGGCCCAAGGTCCCCTGGTTCCCAAGAAAAGT
GTCAGAGCTGGACAAGTGTCATCACCTGGTCACCAAGTTCGACCCTGACCTGGACTTGGACCACCCGGGCTTCTCGGACCAGGTGTACCG
CCAGCGCAGGAAGCTGATTGCTGAGATCGCCTTCCAGTACAGGCACGGCGACCCGATTCCCCGTGTGGAGTACACCGCCGAGGAGATTGC
CACCTGGAAGGAGGTCTACACCACGCTGAAGGGCCTCTACGCCACGCACGCCTGCGGGGAGCACCTGGAGGCCTTTGCTTTGCTGGAGCG
CTTCAGCGGCTACCGGGAAGACAATATCCCCCAGCTGGAGGACGTCTCCCGCTTCCTGAAGGAGCGCACGGGCTTCCAGCTGCGGCCTGT
GGCCGGCCTGCTGTCCGCCCGGGACTTCCTGGCCAGCCTGGCCTTCCGCGTGTTCCAGTGCACCCAGTATATCCGCCACGCGTCCTCGCC
CATGCACTCCCCTGAGCCGGACTGCTGCCACGAGCTGCTGGGGCACGTGCCCATGCTGGCCGACCGCACCTTCGCGCAGTTCTCGCAGGA
CATTGGCCTGGCGTCCCTGGGGGCCTCGGATGAGGAAATTGAGAAGCTGTCCACGCTGTACTGGTTCACGGTGGAGTTCGGGCTGTGTAA
GCAGAACGGGGAGGTGAAGGCCTATGGTGCCGGGCTGCTGTCCTCCTACGGGGAGCTCCTGCACTGCCTGTCTGAGGAGCCTGAGATTCG
GGCCTTCGACCCTGAGGCTGCGGCCGTGCAGCCCTACCAAGACCAGACGTACCAGTCAGTCTACTTCGTGTCTGAGAGCTTCAGTGACGC
CAAGGACAAGCTCAGGAGCTATGCCTCACGCATCCAGCGCCCCTTCTCCGTGAAGTTCGACCCGTACACGCTGGCCATCGACGTGCTGGA
CAGCCCCCAGGCCGTGCGGCGCTCCCTGGAGGGTGTCCAGGATGAGCTGGACACCCTTGCCCATGCGCTGAGTGCCATTGGCTAACTAGT
GGATCCGTCGATAATCAACCTCTGGATTACAAAATTTGTGAAAGATTGACTGGTATTCTTAACTATGTTGCTCCTTTTACGCTATGTGGA
TACGCTGCTTTAATGCCTTTGTATCATGCTATTGCTTCCCGTATGGCTTTCATTTTCTCCTCCTTGTATAAATCCTGGTTGCTGTCTCTT
TATGAGGAGTTGTGGCCCGTTGTCAGGCAACGTGGCGTGGTGTGCACTGTGTTTGCTGACGCAACCCCCACTGGTTGGGGCATTGCCACC
ACCTGTCAGCTCCTTTCCGGGACTTTCGCTTTCCCCCTCCCTATTGCCACGGCGGAACTCATCGCCGCCTGCCTTGCCCGCTGCTGGACA
GGGGCTCGGCTGTTGGGCACTGACAATTCCGTGGTGTTGTCGGGGAAATCATCGTCCTTTCCTTGGCTGCTCGCCTGTGTTGCCACCTGG
ATTCTGCGCGGGACGTCCTTCTGCTACGTCCCTTCGGCCCTCAATCCAGCGGACCTTCCTTCCCGCGGCCTGCTGCCGGCTCTGCGGCCT
CTTCCGCGTCTTCGCCTTCGCCCTCAGACGAGTCGGATCTCCCTTTGGGCCGCCTCCCCGCCTGGAATTCGAGCTCGGTACAGCTTATCG
ATACCGTCGACTTCGAGCAACTTGTTTATTGCAGCTTATAATGGTTACAAATAAAGCAATAGCATCACAAATTTCACAAATAAAGCATTT
TTTTCACTGCATTCTAGTTGTGGTTTGTCCAAACTCATCAATGTATCTTATCATGTCTGGATCGTCTAGCATCGAAGATCCCCCGCTAGT
CCACTCCCTCTCTGCGCGCTCGCTCGCTCACTGAGGCCGGGCGACCAAAGGTCGCCCGACGCCCGGGCTTTGCCCGGGCGGCCTCAGTGA
GCGAGCGAGCGCGCAGAGAGGGACAGATCCGGGCCCGCATGCGTCGACAATTCACTGGCCGTCGTTTTACAACGTCGTGACTGGGAAAAC
CCTGGCGTTACCCAACTTAATCGCCTTGCAGCACATCCCCCTTTCGCCAGCTGGCGTAATAGCGAAGAGGCCCGCACCGATCGCCCTTCC
CAACAGTTGCGCAGCCTGAATGGCGAATGGCGCCTGATGCGGTATTTTCTCCTTACGCATCTGTGCGGTATTTCACACCGCATATGGTGC
ACTCTCAGTACAATCTGCTCTGATGCCGCATAGTTAAGCCAGCCCCGACACCCGCCAACACCCGCTGACGCGCCCTGACGGGCTTGTCTG
CTCCCGGCATCCGCTTACAGACAAGCTGTGACCGTCTCCGGGAGCTGCATGTGTCAGAGGTTTTCACCGTCATCACCGAAACGCGCGAGA
CGAAAGGGCCTCGTGATACGCCTATTTTTATAGGTTAATGTCATGATAATAATGGTTTCTTAGACGTCAGGTGGCACTTTTCGGGGAAAT
GTGCGCGGAACCCCTATTTGTTTATTTTTCTAAATACATTCAAATATGTATCCGCTCATGAGACAATAACCCTGATAAATGCTTCAATAA
TATTGAAAAAGGAAGAGTATGAGTATTCAACATTTCCGTGTCGCCCTTATTCCCTTTTTTGCGGCATTTTGCCTTCCTGTTTTTGCTCAC
CCAGAAACGCTGGTGAAAGTAAAAGATGCTGAAGATCAGTTGGGTGCACGAGTGGGTTACATCGAACTGGATCTCAACAGCGGTAAGATC
CTTGAGAGTTTTCGCCCCGAAGAACGTTTTCCAATGATGAGCACTTTTAAAGTTCTGCTATGTGGCGCGGTATTATCCCGTATTGACGCC
GGGCAAGAGCAACTCGGTCGCCGCATACACTATTCTCAGAATGACTTGGTTGAGTACTCACCAGTCACAGAAAAGCATCTTACGGATGGC
ATGACAGTAAGAGAATTATGCAGTGCTGCCATAACCATGAGTGATAACACTGCGGCCAACTTACTTCTGACAACGATCGGAGGACCGAAG
GAGCTAACCGCTTTTTTGCACAACATGGGGGATCATGTAACTCGCCTTGATCGTTGGGAACCGGAGCTGAATGAAGCCATACCAAACGAC
GAGCGTGACACCACGATGCCTGTAGCAATGGCAACAACGTTGCGCAAACTATTAACTGGCGAACTACTTACTCTAGCTTCCCGGCAACAA
TTAATAGACTGGATGGAGGCGGATAAAGTTGCAGGACCACTTCTGCGCTCGGCCCTTCCGGCTGGCTGGTTTATTGCTGATAAATCTGGA
GCCGGTGAGCGTGGGTCTCGCGGTATCATTGCAGCACTGGGGCCAGATGGTAAGCCCTCCCGTATCGTAGTTATCTACACGACGGGGAGT
CAGGCAACTATGGATGAACGAAATAGACAGATCGCTGAGATAGGTGCCTCACTGATTAAGCATTGGTAACTGTCAGACCAAGTTTACTCA
TATATACTTTAGATTGATTTAAAACTTCATTTTTAATTTAAAAGGATCTAGGTGAAGATCCTTTTTGATAATCTCATGACCAAAATCCCT
TAACGTGAGTTTTCGTTCCACTGAGCGTCAGACCCCGTAGAAAAGATCAAAGGATCTTCTTGAGATCCTTTTTTTCTGCGCGTAATCTGC
TGCTTGCAAACAAAAAAACCACCGCTACCAGCGGTGGTTTGTTTGCCGGATCAAGAGCTACCAACTCTTTTTCCGAAGGTAACTGGCTTC
AGCAGAGCGCAGATACCAAATACTGTTCTTCTAGTGTAGCCGTAGTTAGGCCACCACTTCAAGAACTCTGTAGCACCGCCTACATACCTC
GCTCTGCTAATCCTGTTACCAGTGGCTGCTGCCAGTGGCGATAAGTCGTGTCTTACCGGGTTGGACTCAAGACGATAGTTACCGGATAAG
GCGCAGCGGTCGGGCTGAACGGGGGGTTCGTGCACACAGCCCAGCTTGGAGCGAACGACCTACACCGAACTGAGATACCTACAGCGTGAG
CTATGAGAAAGCGCCACGCTTCCCGAAGGGAGAAAGGCGGACAGGTATCCGGTAAGCGGCAGGGTCGGAACAGGAGAGCGCACGAGGGAG
CTTCCAGGGGGAAACGCCTGGTATCTTTATAGTCCTGTCGGGTTTCGCCACCTCTGACTTGAGCGTCGATTTTTGTGATGCTCGTCAGGG
GGGCGGAGCCTATGGAAAAACGCCAGCAACGCGGCCTTTTTACGGTTCCTGGCCTTTTGCTGGCCTTTTGCTCACATGTTCTTTCCTGCG
TTATCCCCTGATTCTGTGGATAACCGTATTACCGCCTTTGAGTGAGCTGATACCGCTCGCCGCAGCCGAACGACCGAGCGCAGCGAGTCA
GTGAGCGAGGAAGCGGAAGAGCGCCCAATACGCAAACCGCCTCTCCCCGCGCGTTGGCCGATTCATTAATGCAGCTGGCACGACAGGTTT
CCCGACTGGAAAGCGGGCAGTGAGCGCAACGCAATTAATGTGAGTTAGCTCACTCATTAGGCACCCCAGGCTTTACACTTTATGCTTCCG
GCTCGTATGTTGTGTGGAATTGTGAGCGGATAACAATTTCACACAGGAAACAGCTATGACCATGATTACGCCAAGCTCTCGAGATCTAG
SEQ ID NO: 33: pAAo19-scAAV-LP1-GCH1-LP1-tTH:
CAGCAGCTGCGCGCTCGCTCGCTCACTGAGGCCGCCCGGGCAAAGCCCGGGCGTCGGGCGACCTTTGGTCGCCCGGCCTCAGTGAGCGAG
CGAGCGCGCAGAGAGGGAGTGGCCAACTCCATCACTAGGGGTTCCTTGTAGTTAATGATTAACCCGCCATGCTACTTATCTACGTAGCCA
TGCTCTAGAgctgagcCCCTAAAATGGGCAAACATTGCAAGCAGCAAACAGCAAACACACAGCCCTCCCTGCCTGCTGACCTTGGAGCTG
GGGCAGAGGTCAGAGACCTCTCTGGGCCCATGCCACCTCCAACATCCACTCGACCCCTTGGAATTTCGGTGGAGAGGAGCAGAGGTTGTC
CTGGCGTGGTTTAGGTAGTGTGAGAGGGGAATGACTCCTTTCGGTAAGTGCAGTGGAAGCTGTACACTGCCCAGGCAAAGCGTCCGGGCA
GCGTAGGCGGGCGACTCAGATCCCAGCCAGTGGACTTAGCCCCTGTTTGCTCCTCCGATAACTGGGGTGACCTTGGTTAATATTCACCAG
CAGCCTCCCCCGTTGCCCCTCTGGATCCACTGCTTAAATACGGACGAGGACAGGGCCCTGTCTCCTCAGCTTCAGGCACCACCACTGACC
TGGGACAGTGAATCCGGACTCTAAGGTAAATATAAAATTTTTAAGTGTATAATGTGTTAAACTACTGATTCTAATTGTTTCTCTCTTTTA
GATTCCAACCTTTGGAACTGAcctgcaggATTCAAGCTGCTAGCAAGGATCCACCGGTAACATGGAGAAGGGCCCTGTGCGGGCACCGGC
GGAGAAGCCGCGGGGCGCCAGGTGCAGCAATGGGTTCCCCGAGCGGGATCCGCCGCGGCCCGGGCCCAGCAGGCCGGCGGAGAAGCCCCC
GCGGCCCGAGGCCAAGAGCGCGCAGCCCGCGGACGGCTGGAAGGGCGAGCGGCCCCGCAGCGAGGAGGATAACGAGCTGAACCTCCCTAA
CCTGGCAGCCGCCTACTCGTCCATCCTGAGCTCGCTGGGCGAGAACCCCCAGCGGCAAGGGCTGCTCAAGACGCCCTGGAGGGCGGCCTC
GGCCATGCAGTTCTTCACCAAGGGCTACCAGGAGACCATCTCAGATGTCCTAAACGATGCTATATTTGATGAAGATCATGATGAGATGGT
GATTGTGAAGGACATAGACATGTTTTCCATGTGTGAGCATCACTTGGTTCCATTTGTTGGAAAGGTCCATATTGGTTATCTTCCTAACAA
GCAAGTCCTTGGCCTCAGCAAACTTGCGAGGATTGTAGAAATCTATAGTAGAAGACTACAAGTTCAGGAGCGCCTTACAAAACAAATTGC
TGTAGCAATCACGGAAGCCTTGCGGCCTGCTGGAGTCGGGGTAGTGGTTGAAGCAACACACATGTGTATGGTAATGCGAGGTGTACAGAA
AATGAACAGCAAAACTGTGACCAGCACAATGTTGGGTGTGTTCCGGGAGGATCCAAAGACTCGGGAAGAGTTCCTGACTCTCATTAGGAG
CTAATGCATCCCCATCGATGATCCAGACATGATAAGATACATTGATGAGTTTGGACAAACCACAACTAGAATGCAGTGAAAAAAATGCTT
TATTTGTGAAATTTGTGATGCTATTGCTTTATTTGTAACCATTATAAGCTGCAATAAACAAGTTAACAACAACAATTGCATTCATTTTAT
GTTTCAGGTTCAGGGGGAGGTGTGGGAGGTTTTTTAGTCGACCAGATCTGACAAGGTCCCCTAAAATGGGCAAACATTGCAAGCAGCAAA
CAGCAAACACACAGCCCTCCCTGCCTGCTGACCTTGGAGCTGGGGCAGAGGTCAGAGACCTCTCTGGGCCCATGCCACCTCCAACATCCA
CTCGACCCCTTGGAATTTCGGTGGAGAGGAGCAGAGGTTGTCCTGGCGTGGTTTAGGTAGTGTGAGAGGGGAATGACTCCTTTCGGTAAG
TGCAGTGGAAGCTGTACACTGCCCAGGCAAAGCGTCCGGGCAGCGTAGGCGGGCGACTCAGATCCCAGCCAGTGGACTTAGCCCCTGTTT
GCTCCTCCGATAACTGGGGTGACCTTGGTTAATATTCACCAGCAGCCTCCCCCGTTGCCCCTCTGGATCCACTGCTTAAATACGGACGAG
GACAGGGCCCTGTCTCCTCAGCTTCAGGCACCACCACTGACCTGGGACAGTGAATCCGGACTCTAAGGTAAATATAAAATTTTTAAGTGT
ATAATGTGTTAAACTACTGATTCTAATTGTTTCTCTCTTTTAGATTCCAACCTTTGGAACTGATTCGAAATTCAAGCTGCTAGCAAGGAT
CCACCGGTCACCATGAGCCCCGCGGGGCCCAAGGTCCCCTGGTTCCCAAGAAAAGTGTCAGAGCTGGACAAGTGTCATCACCTGGTCACC
AAGTTCGACCCTGACCTGGACTTGGACCACCCGGGCTTCTCGGACCAGGTGTACCGCCAGCGCAGGAAGCTGATTGCTGAGATCGCCTTC
CAGTACAGGCACGGCGACCCGATTCCCCGTGTGGAGTACACCGCCGAGGAGATTGCCACCTGGAAGGAGGTCTACACCACGCTGAAGGGC
CTCTACGCCACGCACGCCTGCGGGGAGCACCTGGAGGCCTTTGCTTTGCTGGAGCGCTTCAGCGGCTACCGGGAAGACAATATCCCCCAG
CTGGAGGACGTCTCCCGCTTCCTGAAGGAGCGCACGGGCTTCCAGCTGCGGCCTGTGGCCGGCCTGCTGTCCGCCCGGGACTTCCTGGCC
AGCCTGGCCTTCCGCGTGTTCCAGTGCACCCAGTATATCCGCCACGCGTCCTCGCCCATGCACTCCCCTGAGCCGGACTGCTGCCACGAG
CTGCTGGGGCACGTGCCCATGCTGGCCGACCGCACCTTCGCGCAGTTCTCGCAGGACATTGGCCTGGCGTCCCTGGGGGCCTCGGATGAG
GAAATTGAGAAGCTGTCCACGCTGTACTGGTTCACGGTGGAGTTCGGGCTGTGTAAGCAGAACGGGGAGGTGAAGGCCTATGGTGCCGGG
CTGCTGTCCTCCTACGGGGAGCTCCTGCACTGCCTGTCTGAGGAGCCTGAGATTCGGGCCTTCGACCCTGAGGCTGCGGCCGTGCAGCCC
TACCAAGACCAGACGTACCAGTCAGTCTACTTCGTGTCTGAGAGCTTCAGTGACGCCAAGGACAAGCTCAGGAGCTATGCCTCACGCATC
CAGCGCCCCTTCTCCGTGAAGTTCGACCCGTACACGCTGGCCATCGACGTGCTGGACAGCCCCCAGGCCGTGCGGCGCTCCCTGGAGGGT
GTCCAGGATGAGCTGGACACCCTTGCCCATGCGCTGAGTGCCATTGGCTAACTAGTGGATCCGTCGATAATCAACCTCTGGATTACAAAA
TTTGTGAAAGATTGACTGGTATTCTTAACTATGTTGCTCCTTTTACGCTATGTGGATACGCTGCTTTAATGCCTTTGTATCATGCTATTG
CTTCCCGTATGGCTTTCATTTTCTCCTCCTTGTATAAATCCTGGTTGCTGTCTCTTTATGAGGAGTTGTGGCCCGTTGTCAGGCAACGTG
GCGTGGTGTGCACTGTGTTTGCTGACGCAACCCCCACTGGTTGGGGCATTGCCACCACCTGTCAGCTCCTTTCCGGGACTTTCGCTTTCC
CCCTCCCTATTGCCACGGCGGAACTCATCGCCGCCTGCCTTGCCCGCTGCTGGACAGGGGCTCGGCTGTTGGGCACTGACAATTCCGTGG
TGTTGTCGGGGAAATCATCGTCCTTTCCTTGGCTGCTCGCCTGTGTTGCCACCTGGATTCTGCGCGGGACGTCCTTCTGCTACGTCCCTT
CGGCCCTCAATCCAGCGGACCTTCCTTCCCGCGGCCTGCTGCCGGCTCTGCGGCCTCTTCCGCGTCTTCGCCTTCGCCCTCAGACGAGTC
GGATCTCCCTTTGGGCCGCCTCCCCGCCTGGAATTCGAGCTCGGTACAGCTTATCGATACCGTCGACTTCGAGCAACTTGTTTATTGCAG
CTTATAATGGTTACAAATAAAGCAATAGCATCACAAATTTCACAAATAAAGCATTTTTTTCACTGCATTCTAGTTGTGGTTTGTCCAAAC
TCATCAATGTATCTTATCATGTCTGGATCGTCTAGCATCGAAGATCCCCCGCATGCTCTAGAGCATGGCTACGTAGATAAGTAGCATGGC
GGGTTAATCATTAACTACAAGGAACCCCTAGTGATGGAGTTGGCCACTCCCTCTCTGCGCGCTCGCTCGCTCACTGAGGCCGGGCGACCA
AAGGTCGCCCGACGCCCGGGCTTTGCCCGGGCGGCCTCAGTGAGCGAGCGAGCGCGCAGCTGGCGTAATAGCGAAGAGGCCCGCACCGAT
CGCCCTTCCCAACAGTTGCGCAGCCTGAATGGCGAATGGAATTCCAGACGATTGAGCGTCAAAATGTAGGTATTTCCATGAGCGTTTTTC
CTGTTGCAATGGCTGGCGGTAATATTGTTCTGGATATTACCAGCAAGGCCGATAGTTTGAGTTCTTCTACTCAGGCAAGTGATGTTATTA
CTAATCAAAGAAGTATTGCGACAACGGTTAATTTGCGTGATGGACAGACTCTTTTACTCGGTGGCCTCACTGATTATAAAAACACTTCTC
AGGATTCTGGCGTACCGTTCCTGTCTAAAATCCCTTTAATCGGCCTCCTGTTTAGCTCCCGCTCTGATTCTAACGAGGAAAGCACGTTAT
ACGTGCTCGTCAAAGCAACCATAGTACGCGCCCTGTAGCGGCGCATTAAGCGCGGCGGGTGTGGTGGTTACGCGCAGCGTGACCGCTACA
CTTGCCAGCGCCCTAGCGCCCGCTCCTTTCGCTTTCTTCCCTTCCTTTCTCGCCACGTTCGCCGGCTTTCCCCGTCAAGCTCTAAATCGG
GGGCTCCCTTTAGGGTTCCGATTTAGTGCTTTACGGCACCTCGACCCCAAAAAACTTGATTAGGGTGATGGTTCACGTAGTGGGCCATCG
CCCTGATAGACGGTTTTTCGCCCTTTGACGTTGGAGTCCACGTTCTTTAATAGTGGACTCTTGTTCCAAACTGGAACAACACTCAACCCT
ATCTCGGTCTATTCTTTTGATTTATAAGGGATTTTGCCGATTTCGGCCTATTGGTTAAAAAATGAGCTGATTTAACAAAAATTTAACGCG
AATTTTAACAAAATATTAACGTTTACAATTTAAATATTTGCTTATACAATCTTCCTGTTTTTGGGGCTTTTCTGATTATCAACCGGGGTA
CATATGATTGACATGCTAGTTTTACGATTACCGTTCATCGATTCTCTTGTTTGCTCCAGACTCTCAGGCAATGACCTGATAGCCTTTGTA
GAGACCTCTCAAAAATAGCTACCCTCTCCGGCATGAATTTATCAGCTAGAACGGTTGAATATCATATTGATGGTGATTTGACTGTCTCCG
GCCTTTCTCACCCGTTTGAATCTTTACCTACACATTACTCAGGCATTGCATTTAAAATATATGAGGGTTCTAAAAATTTTTATCCTTGCG
TTGAAATAAAGGCTTCTCCCGCAAAAGTATTACAGGGTCATAATGTTTTTGGTACAACCGATTTAGCTTTATGCTCTGAGGCTTTATTGC
TTAATTTTGCTAATTCTTTGCCTTGCCTGTATGATTTATTGGATGTTGGAATTCCTGATGCGGTATTTTCTCCTTACGCATCTGTGCGGT
ATTTCACACCGCATATGGTGCACTCTCAGTACAATCTGCTCTGATGCCGCATAGTTAAGCCAGCCCCGACACCCGCCAACACCCGCTGAC
GCGCCCTGACGGGCTTGTCTGCTCCCGGCATCCGCTTACAGACAAGCTGTGACCGTCTCCGGGAGCTGCATGTGTCAGAGGTTTTCACCG
TCATCACCGAAACGCGCGAGACGAAAGGGCCTCGTGATACGCCTATTTTTATAGGTTAATGTCATGATAATAATGGTTTCTTAGACGTCA
GGTGGCACTTTTCGGGGAAATGTGCGCGGAACCCCTATTTGTTTATTTTTCTAAATACATTCAAATATGTATCCGCTCATGAGACAATAA
CCCTGATAAATGCTTCAATAATATTGAAAAAGGAAGAGTATGAGTATTCAACATTTCCGTGTCGCCCTTATTCCCTTTTTTGCGGCATTT
TGCCTTCCTGTTTTTGCTCACCCAGAAACGCTGGTGAAAGTAAAAGATGCTGAAGATCAGTTGGGTGCACGAGTGGGTTACATCGAACTG
GATCTCAACAGCGGTAAGATCCTTGAGAGTTTTCGCCCCGAAGAACGTTTTCCAATGATGAGCACTTTTAAAGTTCTGCTATGTGGCGCG
GTATTATCCCGTATTGACGCCGGGCAAGAGCAACTCGGTCGCCGCATACACTATTCTCAGAATGACTTGGTTGAGTACTCACCAGTCACA
GAAAAGCATCTTACGGATGGCATGACAGTAAGAGAATTATGCAGTGCTGCCATAACCATGAGTGATAACACTGCGGCCAACTTACTTCTG
ACAACGATCGGAGGACCGAAGGAGCTAACCGCTTTTTTGCACAACATGGGGGATCATGTAACTCGCCTTGATCGTTGGGAACCGGAGCTG
AATGAAGCCATACCAAACGACGAGCGTGACACCACGATGCCTGTAGCAATGGCAACAACGTTGCGCAAACTATTAACTGGCGAACTACTT
ACTCTAGCTTCCCGGCAACAATTAATAGACTGGATGGAGGCGGATAAAGTTGCAGGACCACTTCTGCGCTCGGCCCTTCCGGCTGGCTGG
TTTATTGCTGATAAATCTGGAGCCGGTGAGCGTGGGTCTCGCGGTATCATTGCAGCACTGGGGCCAGATGGTAAGCCCTCCCGTATCGTA
GTTATCTACACGACGGGGAGTCAGGCAACTATGGATGAACGAAATAGACAGATCGCTGAGATAGGTGCCTCACTGATTAAGCATTGGTAA
CTGTCAGACCAAGTTTACTCATATATACTTTAGATTGATTTAAAACTTCATTTTTAATTTAAAAGGATCTAGGTGAAGATCCTTTTTGAT
AATCTCATGACCAAAATCCCTTAACGTGAGTTTTCGTTCCACTGAGCGTCAGACCCCGTAGAAAAGATCAAAGGATCTTCTTGAGATCCT
TTTTTTCTGCGCGTAATCTGCTGCTTGCAAACAAAAAAACCACCGCTACCAGCGGTGGTTTGTTTGCCGGATCAAGAGCTACCAACTCTT
TTTCCGAAGGTAACTGGCTTCAGCAGAGCGCAGATACCAAATACTGTCCTTCTAGTGTAGCCGTAGTTAGGCCACCACTTCAAGAACTCT
GTAGCACCGCCTACATACCTCGCTCTGCTAATCCTGTTACCAGTGGCTGCTGCCAGTGGCGATAAGTCGTGTCTTACCGGGTTGGACTCA
AGACGATAGTTACCGGATAAGGCGCAGCGGTCGGGCTGAACGGGGGGTTCGTGCACACAGCCCAGCTTGGAGCGAACGACCTACACCGAA
CTGAGATACCTACAGCGTGAGCTATGAGAAAGCGCCACGCTTCCCGAAGGGAGAAAGGCGGACAGGTATCCGGTAAGCGGCAGGGTCGGA
ACAGGAGAGCGCACGAGGGAGCTTCCAGGGGGAAACGCCTGGTATCTTTATAGTCCTGTCGGGTTTCGCCACCTCTGACTTGAGCGTCGA
TTTTTGTGATGCTCGTCAGGGGGGCGGAGCCTATGGAAAAACGCCAGCAACGCGGCCTTTTTACGGTTCCTGGCCTTTTGCTGGCCTTTT
GCTCACATGTTCTTTCCTGCGTTATCCCCTGATTCTGTGGATAACCGTATTACCGCCTTTGAGTGAGCTGATACCGCTCGCCGCAGCCGA
ACGACCGAGCGCAGCGAGTCAGTGAGCGAGGAAGCGGAAGAGCGCCCAATACGCAAACCGCCTCTCCCCGCGCGTTGGCCGATTCATTAA
TGC
SEQ ID NO: 34: pAA010 scAAV-LP1-tTH
AAAGCTTCCCGGGGGGATCTGGGCCACTCCCTCTCTGCGCGCTCGCTCGCTCACTGAGGCCGGGCGACCAAAGGTCGCCCGACGCCCGGG
CTTTGCCCGGGCGGCCTCAGTGAGCGAGCGAGCGCGCAGAGAGGGAGTGGCCAACTCCATCACTAGGGGTTCCTGGAGGGGTGGAGTCGT
GACCCCTAAAATGGGCAAACATTGCAAGCAGCAAACAGCAAACACACAGCCCTCCCTGCCTGCTGACCTTGGAGCTGGGGCAGAGGTCAG
AGACCTCTCTGGGCCCATGCCACCTCCAACATCCACTCGACCCCTTGGAATTTCGGTGGAGAGGAGCAGAGGTTGTCCTGGCGTGGTTTA
GGTAGTGTGAGAGGGGAATGACTCCTTTCGGTAAGTGCAGTGGAAGCTGTACACTGCCCAGGCAAAGCGTCCGGGCAGCGTAGGCGGGCG
ACTCAGATCCCAGCCAGTGGACTTAGCCCCTGTTTGCTCCTCCGATAACTGGGGTGACCTTGGTTAATATTCACCAGCAGCCTCCCCCGT
TGCCCCTCTGGATCCACTGCTTAAATACGGACGAGGACAGGGCCCTGTCTCCTCAGCTTCAGGCACCACCACTGACCTGGGACAGTGAAT
CCGGACTCTAAGGTAAATATAAAATTTTTAAGTGTATAATGTGTTAAACTACTGATTCTAATTGTTTCTCTCTTTTAGATTCCAACCTTT
GGAACTGAATTCTAGCATGAGCCCCGCGGGGCCCAAGGTCCCCTGGTTCCCAAGAAAAGTGTCAGAGCTGGACAAGTGTCATCACCTGGT
CACCAAGTTCGACCCTGACCTGGACTTGGACCACCCGGGCTTCTCGGACCAGGTGTACCGCCAGCGCAGGAAGCTGATTGCTGAGATCGC
CTTCCAGTACAGGCACGGCGACCCGATTCCCCGTGTGGAGTACACCGCCGAGGAGATTGCCACCTGGAAGGAGGTCTACACCACGCTGAA
GGGCCTCTACGCCACGCACGCCTGCGGGGAGCACCTGGAGGCCTTTGCTTTGCTGGAGCGCTTCAGCGGCTACCGGGAAGACAATATCCC
CCAGCTGGAGGACGTCTCCCGCTTCCTGAAGGAGCGCACGGGCTTCCAGCTGCGGCCTGTGGCCGGCCTGCTGTCCGCCCGGGACTTCCT
GGCCAGCCTGGCCTTCCGCGTGTTCCAGTGCACCCAGTATATCCGCCACGCGTCCTCGCCCATGCACTCCCCTGAGCCGGACTGCTGCCA
CGAGCTGCTGGGGCACGTGCCCATGCTGGCCGACCGCACCTTCGCGCAGTTCTCGCAGGACATTGGCCTGGCGTCCCTGGGGGCCTCGGA
TGAGGAAATTGAGAAGCTGTCCACGCTGTACTGGTTCACGGTGGAGTTCGGGCTGTGTAAGCAGAACGGGGAGGTGAAGGCCTATGGTGC
CGGGCTGCTGTCCTCCTACGGGGAGCTCCTGCACTGCCTGTCTGAGGAGCCTGAGATTCGGGCCTTCGACCCTGAGGCTGCGGCCGTGCA
GCCCTACCAAGACCAGACGTACCAGTCAGTCTACTTCGTGTCTGAGAGCTTCAGTGACGCCAAGGACAAGCTCAGGAGCTATGCCTCACG
CATCCAGCGCCCCTTCTCCGTGAAGTTCGACCCGTACACGCTGGCCATCGACGTGCTGGACAGCCCCCAGGCCGTGCGGCGCTCCCTGGA
GGGTGTCCAGGATGAGCTGGACACCCTTGCCCATGCGCTGAGTGCCATTGGCTAACTAGTGGATCCGTCGATAATCAACCTCTGGATTAC
AAAATTTGTGAAAGATTGACTGGTATTCTTAACTATGTTGCTCCTTTTACGCTATGTGGATACGCTGCTTTAATGCCTTTGTATCATGCT
ATTGCTTCCCGTATGGCTTTCATTTTCTCCTCCTTGTATAAATCCTGGTTGCTGTCTCTTTATGAGGAGTTGTGGCCCGTTGTCAGGCAA
CGTGGCGTGGTGTGCACTGTGTTTGCTGACGCAACCCCCACTGGTTGGGGCATTGCCACCACCTGTCAGCTCCTTTCCGGGACTTTCGCT
TTCCCCCTCCCTATTGCCACGGCGGAACTCATCGCCGCCTGCCTTGCCCGCTGCTGGACAGGGGCTCGGCTGTTGGGCACTGACAATTCC
GTGGTGTTGTCGGGGAAATCATCGTCCTTTCCTTGGCTGCTCGCCTGTGTTGCCACCTGGATTCTGCGCGGGACGTCCTTCTGCTACGTC
CCTTCGGCCCTCAATCCAGCGGACCTTCCTTCCCGCGGCCTGCTGCCGGCTCTGCGGCCTCTTCCGCGTCTTCGCCTTCGCCCTCAGACG
AGTCGGATCTCCCTTTGGGCCGCCTCCCCGCCTGGAATTCGAGCTCGGTACAGCTTATCGATACCGTCGACTTCGAGCAACTTGTTTATT
GCAGCTTATAATGGTTACAAATAAAGCAATAGCATCACAAATTTCACAAATAAAGCATTTTTTTCACTGCATTCTAGTTGTGGTTTGTCC
AAACTCATCAATGTATCTTATCATGTCTGGATCGTCTAGCATCGAAGATCCCCCGCTAGTCCACTCCCTCTCTGCGCGCTCGCTCGCTCA
CTGAGGCCGGGCGACCAAAGGTCGCCCGACGCCCGGGCTTTGCCCGGGCGGCCTCAGTGAGCGAGCGAGCGCGCAGAGAGGGACAGATCC
GGGCCCGCATGCGTCGACAATTCACTGGCCGTCGTTTTACAACGTCGTGACTGGGAAAACCCTGGCGTTACCCAACTTAATCGCCTTGCA
GCACATCCCCCTTTCGCCAGCTGGCGTAATAGCGAAGAGGCCCGCACCGATCGCCCTTCCCAACAGTTGCGCAGCCTGAATGGCGAATGG
CGCCTGATGCGGTATTTTCTCCTTACGCATCTGTGCGGTATTTCACACCGCATATGGTGCACTCTCAGTACAATCTGCTCTGATGCCGCA
TAGTTAAGCCAGCCCCGACACCCGCCAACACCCGCTGACGCGCCCTGACGGGCTTGTCTGCTCCCGGCATCCGCTTACAGACAAGCTGTG
ACCGTCTCCGGGAGCTGCATGTGTCAGAGGTTTTCACCGTCATCACCGAAACGCGCGAGACGAAAGGGCCTCGTGATACGCCTATTTTTA
TAGGTTAATGTCATGATAATAATGGTTTCTTAGACGTCAGGTGGCACTTTTCGGGGAAATGTGCGCGGAACCCCTATTTGTTTATTTTTC
TAAATACATTCAAATATGTATCCGCTCATGAGACAATAACCCTGATAAATGCTTCAATAATATTGAAAAAGGAAGAGTATGAGTATTCAA
CATTTCCGTGTCGCCCTTATTCCCTTTTTTGCGGCATTTTGCCTTCCTGTTTTTGCTCACCCAGAAACGCTGGTGAAAGTAAAAGATGCT
GAAGATCAGTTGGGTGCACGAGTGGGTTACATCGAACTGGATCTCAACAGCGGTAAGATCCTTGAGAGTTTTCGCCCCGAAGAACGTTTT
CCAATGATGAGCACTTTTAAAGTTCTGCTATGTGGCGCGGTATTATCCCGTATTGACGCCGGGCAAGAGCAACTCGGTCGCCGCATACAC
TATTCTCAGAATGACTTGGTTGAGTACTCACCAGTCACAGAAAAGCATCTTACGGATGGCATGACAGTAAGAGAATTATGCAGTGCTGCC
ATAACCATGAGTGATAACACTGCGGCCAACTTACTTCTGACAACGATCGGAGGACCGAAGGAGCTAACCGCTTTTTTGCACAACATGGGG
GATCATGTAACTCGCCTTGATCGTTGGGAACCGGAGCTGAATGAAGCCATACCAAACGACGAGCGTGACACCACGATGCCTGTAGCAATG
GCAACAACGTTGCGCAAACTATTAACTGGCGAACTACTTACTCTAGCTTCCCGGCAACAATTAATAGACTGGATGGAGGCGGATAAAGTT
GCAGGACCACTTCTGCGCTCGGCCCTTCCGGCTGGCTGGTTTATTGCTGATAAATCTGGAGCCGGTGAGCGTGGGTCTCGCGGTATCATT
GCAGCACTGGGGCCAGATGGTAAGCCCTCCCGTATCGTAGTTATCTACACGACGGGGAGTCAGGCAACTATGGATGAACGAAATAGACAG
ATCGCTGAGATAGGTGCCTCACTGATTAAGCATTGGTAACTGTCAGACCAAGTTTACTCATATATACTTTAGATTGATTTAAAACTTCAT
TTTTAATTTAAAAGGATCTAGGTGAAGATCCTTTTTGATAATCTCATGACCAAAATCCCTTAACGTGAGTTTTCGTTCCACTGAGCGTCA
GACCCCGTAGAAAAGATCAAAGGATCTTCTTGAGATCCTTTTTTTCTGCGCGTAATCTGCTGCTTGCAAACAAAAAAACCACCGCTACCA
GCGGTGGTTTGTTTGCCGGATCAAGAGCTACCAACTCTTTTTCCGAAGGTAACTGGCTTCAGCAGAGCGCAGATACCAAATACTGTTCTT
CTAGTGTAGCCGTAGTTAGGCCACCACTTCAAGAACTCTGTAGCACCGCCTACATACCTCGCTCTGCTAATCCTGTTACCAGTGGCTGCT
GCCAGTGGCGATAAGTCGTGTCTTACCGGGTTGGACTCAAGACGATAGTTACCGGATAAGGCGCAGCGGTCGGGCTGAACGGGGGGTTCG
TGCACACAGCCCAGCTTGGAGCGAACGACCTACACCGAACTGAGATACCTACAGCGTGAGCTATGAGAAAGCGCCACGCTTCCCGAAGGG
AGAAAGGCGGACAGGTATCCGGTAAGCGGCAGGGTCGGAACAGGAGAGCGCACGAGGGAGCTTCCAGGGGGAAACGCCTGGTATCTTTAT
AGTCCTGTCGGGTTTCGCCACCTCTGACTTGAGCGTCGATTTTTGTGATGCTCGTCAGGGGGGCGGAGCCTATGGAAAAACGCCAGCAAC
GCGGCCTTTTTACGGTTCCTGGCCTTTTGCTGGCCTTTTGCTCACATGTTCTTTCCTGCGTTATCCCCTGATTCTGTGGATAACCGTATT
ACCGCCTTTGAGTGAGCTGATACCGCTCGCCGCAGCCGAACGACCGAGCGCAGCGAGTCAGTGAGCGAGGAAGCGGAAGAGCGCCCAATA
CGCAAACCGCCTCTCCCCGCGCGTTGGCCGATTCATTAATGCAGCTGGCACGACAGGTTTCCCGACTGGAAAGCGGGCAGTGAGCGCAAC
GCAATTAATGTGAGTTAGCTCACTCATTAGGCACCCCAGGCTTTACACTTTATGCTTCCGGCTCGTATGTTGTGTGGAATTGTGAGCGGA
TAACAATTTCACACAGGAAACAGCTATGACCATGATTACGCCAAGCTCTCGAGATCTAG
SEQ ID NO: 35: pAA009 scAAV-LP1-GCH1
AAAGCTTCCCGGGGGGATCTGGGCCACTCCCTCTCTGCGCGCTCGCTCGCTCACTGAGGCCGGGCGACCAAAGGTCGCCCGACGCCCGGG
CTTTGCCCGGGCGGCCTCAGTGAGCGAGCGAGCGCGCAGAGAGGGAGTGGCCAACTCCATCACTAGGGGTTCCTGGAGGGGTGGAGTCGT
GACCCCTAAAATGGGCAAACATTGCAAGCAGCAAACAGCAAACACACAGCCCTCCCTGCCTGCTGACCTTGGAGCTGGGGCAGAGGTCAG
AGACCTCTCTGGGCCCATGCCACCTCCAACATCCACTCGACCCCTTGGAATTTCGGTGGAGAGGAGCAGAGGTTGTCCTGGCGTGGTTTA
GGTAGTGTGAGAGGGGAATGACTCCTTTCGGTAAGTGCAGTGGAAGCTGTACACTGCCCAGGCAAAGCGTCCGGGCAGCGTAGGCGGGCG
ACTCAGATCCCAGCCAGTGGACTTAGCCCCTGTTTGCTCCTCCGATAACTGGGGTGACCTTGGTTAATATTCACCAGCAGCCTCCCCCGT
TGCCCCTCTGGATCCACTGCTTAAATACGGACGAGGACAGGGCCCTGTCTCCTCAGCTTCAGGCACCACCACTGACCTGGGACAGTGAAT
CCGGACTCTAAGGTAAATATAAAATTTTTAAGTGTATAATGTGTTAAACTACTGATTCTAATTGTTTCTCTCTTTTAGATTCCAACCTTT
GGAACTGAATTctagcATGGAGAAGGGCCCTGTGCGGGCACCGGCGGAGAAGCCGCGGGGCGCCAGGTGCAGCAATGGGTTCCCCGAGCG
GGATCCGCCGCGGCCCGGGCCCAGCAGGCCGGCGGAGAAGCCCCCGCGGCCCGAGGCCAAGAGCGCGCAGCCCGCGGACGGCTGGAAGGG
CGAGCGGCCCCGCAGCGAGGAGGATAACGAGCTGAACCTCCCTAACCTGGCAGCCGCCTACTCGTCCATCCTGAGCTCGCTGGGCGAGAA
CCCCCAGCGGCAAGGGCTGCTCAAGACGCCCTGGAGGGCGGCCTCGGCCATGCAGTTCTTCACCAAGGGCTACCAGGAGACCATCTCAGA
TGTCCTAAACGATGCTATATTTGATGAAGATCATGATGAGATGGTGATTGTGAAGGACATAGACATGTTTTCCATGTGTGAGCATCACTT
GGTTCCATTTGTTGGAAAGGTCCATATTGGTTATCTTCCTAACAAGCAAGTCCTTGGCCTCAGCAAACTTGCGAGGATTGTAGAAATCTA
TAGTAGAAGACTACAAGTTCAGGAGCGCCTTACAAAACAAATTGCTGTAGCAATCACGGAAGCCTTGCGGCCTGCTGGAGTCGGGGTAGT
GGTTGAAGCAACACACATGTGTATGGTAATGCGAGGTGTACAGAAAATGAACAGCAAAACTGTGACCAGCACAATGTTGGGTGTGTTCCG
GGAGGATCCAAAGACTCGGGAAGAGTTCCTGACTCTCATTAGGAGCTAATGCATCCCCATCGATGATCCAGACATGATAAGATACATTGA
TGAGTTTGGACAAACCACAACTAGAATGCAGTGAAAAAAATGCTTTATTTGTGAAATTTGTGATGCTATTGCTTTATTTGTAACCATTAT
AAGCTGCAATAAACAAGTTAACAACAACAATTGCATTCATTTTATGTTTCAGGTTCAGGGGGAGGTGTGGGAGGTTTTTTAGTCGACCgC
TAGTCCACTCCCTCTCTGCGCGCTCGCTCGCTCACTGAGGCCGGGCGACCAAAGGTCGCCCGACGCCCGGGCTTTGCCCGGGCGGCCTCA
GTGAGCGAGCGAGCGCGCAGAGAGGGACAGATCCGGGCCCGCATGCGTCGACAATTCACTGGCCGTCGTTTTACAACGTCGTGACTGGGA
AAACCCTGGCGTTACCCAACTTAATCGCCTTGCAGCACATCCCCCTTTCGCCAGCTGGCGTAATAGCGAAGAGGCCCGCACCGATCGCCC
TTCCCAACAGTTGCGCAGCCTGAATGGCGAATGGCGCCTGATGCGGTATTTTCTCCTTACGCATCTGTGCGGTATTTCACACCGCATATG
GTGCACTCTCAGTACAATCTGCTCTGATGCCGCATAGTTAAGCCAGCCCCGACACCCGCCAACACCCGCTGACGCGCCCTGACGGGCTTG
TCTGCTCCCGGCATCCGCTTACAGACAAGCTGTGACCGTCTCCGGGAGCTGCATGTGTCAGAGGTTTTCACCGTCATCACCGAAACGCGC
GAGACGAAAGGGCCTCGTGATACGCCTATTTTTATAGGTTAATGTCATGATAATAATGGTTTCTTAGACGTCAGGTGGCACTTTTCGGGG
AAATGTGCGCGGAACCCCTATTTGTTTATTTTTCTAAATACATTCAAATATGTATCCGCTCATGAGACAATAACCCTGATAAATGCTTCA
ATAATATTGAAAAAGGAAGAGTATGAGTATTCAACATTTCCGTGTCGCCCTTATTCCCTTTTTTGCGGCATTTTGCCTTCCTGTTTTTGC
TCACCCAGAAACGCTGGTGAAAGTAAAAGATGCTGAAGATCAGTTGGGTGCACGAGTGGGTTACATCGAACTGGATCTCAACAGCGGTAA
GATCCTTGAGAGTTTTCGCCCCGAAGAACGTTTTCCAATGATGAGCACTTTTAAAGTTCTGCTATGTGGCGCGGTATTATCCCGTATTGA
CGCCGGGCAAGAGCAACTCGGTCGCCGCATACACTATTCTCAGAATGACTTGGTTGAGTACTCACCAGTCACAGAAAAGCATCTTACGGA
TGGCATGACAGTAAGAGAATTATGCAGTGCTGCCATAACCATGAGTGATAACACTGCGGCCAACTTACTTCTGACAACGATCGGAGGACC
GAAGGAGCTAACCGCTTTTTTGCACAACATGGGGGATCATGTAACTCGCCTTGATCGTTGGGAACCGGAGCTGAATGAAGCCATACCAAA
CGACGAGCGTGACACCACGATGCCTGTAGCAATGGCAACAACGTTGCGCAAACTATTAACTGGCGAACTACTTACTCTAGCTTCCCGGCA
ACAATTAATAGACTGGATGGAGGCGGATAAAGTTGCAGGACCACTTCTGCGCTCGGCCCTTCCGGCTGGCTGGTTTATTGCTGATAAATC
TGGAGCCGGTGAGCGTGGGTCTCGCGGTATCATTGCAGCACTGGGGCCAGATGGTAAGCCCTCCCGTATCGTAGTTATCTACACGACGGG
GAGTCAGGCAACTATGGATGAACGAAATAGACAGATCGCTGAGATAGGTGCCTCACTGATTAAGCATTGGTAACTGTCAGACCAAGTTTA
CTCATATATACTTTAGATTGATTTAAAACTTCATTTTTAATTTAAAAGGATCTAGGTGAAGATCCTTTTTGATAATCTCATGACCAAAAT
CCCTTAACGTGAGTTTTCGTTCCACTGAGCGTCAGACCCCGTAGAAAAGATCAAAGGATCTTCTTGAGATCCTTTTTTTCTGCGCGTAAT
CTGCTGCTTGCAAACAAAAAAACCACCGCTACCAGCGGTGGTTTGTTTGCCGGATCAAGAGCTACCAACTCTTTTTCCGAAGGTAACTGG
CTTCAGCAGAGCGCAGATACCAAATACTGTTCTTCTAGTGTAGCCGTAGTTAGGCCACCACTTCAAGAACTCTGTAGCACCGCCTACATA
CCTCGCTCTGCTAATCCTGTTACCAGTGGCTGCTGCCAGTGGCGATAAGTCGTGTCTTACCGGGTTGGACTCAAGACGATAGTTACCGGA
TAAGGCGCAGCGGTCGGGCTGAACGGGGGGTTCGTGCACACAGCCCAGCTTGGAGCGAACGACCTACACCGAACTGAGATACCTACAGCG
TGAGCTATGAGAAAGCGCCACGCTTCCCGAAGGGAGAAAGGCGGACAGGTATCCGGTAAGCGGCAGGGTCGGAACAGGAGAGCGCACGAG
GGAGCTTCCAGGGGGAAACGCCTGGTATCTTTATAGTCCTGTCGGGTTTCGCCACCTCTGACTTGAGCGTCGATTTTTGTGATGCTCGTC
AGGGGGGCGGAGCCTATGGAAAAACGCCAGCAACGCGGCCTTTTTACGGTTCCTGGCCTTTTGCTGGCCTTTTGCTCACATGTTCTTTCC
TGCGTTATCCCCTGATTCTGTGGATAACCGTATTACCGCCTTTGAGTGAGCTGATACCGCTCGCCGCAGCCGAACGACCGAGCGCAGCGA
GTCAGTGAGCGAGGAAGCGGAAGAGCGCCCAATACGCAAACCGCCTCTCCCCGCGCGTTGGCCGATTCATTAATGCAGCTGGCACGACAG
GTTTCCCGACTGGAAAGCGGGCAGTGAGCGCAACGCAATTAATGTGAGTTAGCTCACTCATTAGGCACCCCAGGCTTTACACTTTATGCT
TCCGGCTCGTATGTTGTGTGGAATTGTGAGCGGATAACAATTTCACACAGGAAACAGCTATGACCATGATTACGCCAAGCTCTCGAGATC
TAG
SEQ ID NO: 36: scAAV-LP1-hFIXco
AAAGCTTCCCGGGGGGATCTGGGCCACTCCCTCTCTGCGCGCTCGCTCGCTCACTGAGGCCGGGCGACCAAAGGTCGCCCGACGCCCGGG
CTTTGCCCGGGCGGCCTCAGTGAGCGAGCGAGCGCGCAGAGAGGGAGTGGCCAACTCCATCACTAGGGGTTCCTGGAGGGGTGGAGTCGT
GACCCCTAAAATGGGCAAACATTGCAAGCAGCAAACAGCAAACACACAGCCCTCCCTGCCTGCTGACCTTGGAGCTGGGGCAGAGGTCAG
AGACCTCTCTGGGCCCATGCCACCTCCAACATCCACTCGACCCCTTGGAATTTCGGTGGAGAGGAGCAGAGGTTGTCCTGGCGTGGTTTA
GGTAGTGTGAGAGGGGAATGACTCCTTTCGGTAAGTGCAGTGGAAGCTGTACACTGCCCAGGCAAAGCGTCCGGGCAGCGTAGGCGGGCG
ACTCAGATCCCAGCCAGTGGACTTAGCCCCTGTTTGCTCCTCCGATAACTGGGGTGACCTTGGTTAATATTCACCAGCAGCCTCCCCCGT
TGCCCCTCTGGATCCACTGCTTAAATACGGACGAGGACAGGGCCCTGTCTCCTCAGCTTCAGGCACCACCACTGACCTGGGACAGTGAAT
CCGGACTCTAAGGTAAATATAAAATTTTTAAGTGTATAATGTGTTAAACTACTGATTCTAATTGTTTCTCTCTTTTAGATTCCAACCTTT
GGAACTGAATTCTAGACCACCATGCAGAGGGTGAACATGATCATGGCTGAGAGCCCTGGCCTGATCACCATCTGCCTGCTGGGCTACCTG
CTGTCTGCTGAGTGCACTGTGTTCCTGGACCATGAGAATGCCAACAAGATCCTGAACAGGCCCAAGAGATACAACTCTGGCAAGCTGGAG
GAGTTTGTGCAGGGCAACCTGGAGAGGGAGTGCATGGAGGAGAAGTGCAGCTTTGAGGAGGCCAGGGAGGTGTTTGAGAACACTGAGAGG
ACCACTGAGTTCTGGAAGCAGTATGTGGATGGGGACCAGTGTGAGAGCAACCCCTGCCTGAATGGGGGCAGCTGCAAGGATGACATCAAC
AGCTATGAGTGCTGGTGCCCCTTTGGCTTTGAGGGCAAGAACTGTGAGCTGGATGTGACCTGCAACATCAAGAATGGCAGATGTGAGCAG
TTCTGCAAGAACTCTGCTGACAACAAGGTGGTGTGCAGCTGCACTGAGGGCTACAGGCTGGCTGAGAACCAGAAGAGCTGTGAGCCTGCT
GTGCCATTCCCATGTGGCAGAGTGTCTGTGAGCCAGACCAGCAAGCTGACCAGGGCTGAGGCTGTGTTCCCTGATGTGGACTATGTGAAC
AGCACTGAGGCTGAAACCATCCTGGACAACATCACCCAGAGCACCCAGAGCTTCAATGACTTCACCAGGGTGGTGGGGGGGGAGGATGCC
AAGCCTGGCCAGTTCCCCTGGCAAGTGGTGCTGAATGGCAAGGTGGATGCCTTCTGTGGGGGCAGCATTGTGAATGAGAAGTGGATTGTG
ACTGCTGCCCACTGTGTGGAGACTGGGGTGAAGATCACTGTGGTGGCTGGGGAGCACAACATTGAGGAGACTGAGCACACTGAGCAGAAG
AGGAATGTGATCAGGATCATCCCCCACCACAACTACAATGCTGCCATCAACAAGTACAACCATGACATTGCCCTGCTGGAGCTGGATGAG
CCCCTGGTGCTGAACAGCTATGTGACCCCCATCTGCATTGCTGACAAGGAGTACACCAACATCTTCCTGAAGTTTGGCTCTGGCTATGTG
TCTGGCTGGGGCAGGGTGTTCCACAAGGGCAGGTCTGCCCTGGTGCTGCAGTACCTGAGGGTGCCCCTGGTGGACAGGGCCACCTGCCTG
AGGAGCACCAAGTTCACCATCTACAACAACATGTTCTGTGCTGGCTTCCATGAGGGGGGCAGGGACAGCTGCCAGGGGGACTCTGGGGGC
CCCCATGTGACTGAGGTGGAGGGCACCAGCTTCCTGACTGGCATCATCAGCTGGGGGGAGGAGTGTGCCATGAAGGGCAAGTATGGCATC
TACACCAAAGTCTCCAGATATGTGAACTGGATCAAGGAGAAGACCAAGCTGACCTGACTCGATGCTTTATTTGTGAAATTTGTGATGCTA
TTGCTTTATTTGTAACCATTATAAGCTGCAATAAACAAGTTAACAACAACAATTGCATTCATTTTATGTTTCAGGTTCAGGGGGAGGTGT
GGGAGGTTTTTTAAACTAGTCCACTCCCTCTCTGCGCGCTCGCTCGCTCACTGAGGCCGGGCGACCAAAGGTCGCCCGACGCCCGGGCTT
TGCCCGGGCGGCCTCAGTGAGCGAGCGAGCGCGCAGAGAGGGACAGATCCGGGCCCGCATGCGTCGACAATTCACTGGCCGTCGTTTTAC
AACGTCGTGACTGGGAAAACCCTGGCGTTACCCAACTTAATCGCCTTGCAGCACATCCCCCTTTCGCCAGCTGGCGTAATAGCGAAGAGG
CCCGCACCGATCGCCCTTCCCAACAGTTGCGCAGCCTGAATGGCGAATGGCGCCTGATGCGGTATTTTCTCCTTACGCATCTGTGCGGTA
TTTCACACCGCATATGGTGCACTCTCAGTACAATCTGCTCTGATGCCGCATAGTTAAGCCAGCCCCGACACCCGCCAACACCCGCTGACG
CGCCCTGACGGGCTTGTCTGCTCCCGGCATCCGCTTACAGACAAGCTGTGACCGTCTCCGGGAGCTGCATGTGTCAGAGGTTTTCACCGT
CATCACCGAAACGCGCGAGACGAAAGGGCCTCGTGATACGCCTATTTTTATAGGTTAATGTCATGATAATAATGGTTTCTTAGACGTCAG
GTGGCACTTTTCGGGGAAATGTGCGCGGAACCCCTATTTGTTTATTTTTCTAAATACATTCAAATATGTATCCGCTCATGAGACAATAAC
CCTGATAAATGCTTCAATAATATTGAAAAAGGAAGAGTATGAGTATTCAACATTTCCGTGTCGCCCTTATTCCCTTTTTTGCGGCATTTT
GCCTTCCTGTTTTTGCTCACCCAGAAACGCTGGTGAAAGTAAAAGATGCTGAAGATCAGTTGGGTGCACGAGTGGGTTACATCGAACTGG
ATCTCAACAGCGGTAAGATCCTTGAGAGTTTTCGCCCCGAAGAACGTTTTCCAATGATGAGCACTTTTAAAGTTCTGCTATGTGGCGCGG
TATTATCCCGTATTGACGCCGGGCAAGAGCAACTCGGTCGCCGCATACACTATTCTCAGAATGACTTGGTTGAGTACTCACCAGTCACAG
AAAAGCATCTTACGGATGGCATGACAGTAAGAGAATTATGCAGTGCTGCCATAACCATGAGTGATAACACTGCGGCCAACTTACTTCTGA
CAACGATCGGAGGACCGAAGGAGCTAACCGCTTTTTTGCACAACATGGGGGATCATGTAACTCGCCTTGATCGTTGGGAACCGGAGCTGA
ATGAAGCCATACCAAACGACGAGCGTGACACCACGATGCCTGTAGCAATGGCAACAACGTTGCGCAAACTATTAACTGGCGAACTACTTA
CTCTAGCTTCCCGGCAACAATTAATAGACTGGATGGAGGCGGATAAAGTTGCAGGACCACTTCTGCGCTCGGCCCTTCCGGCTGGCTGGT
TTATTGCTGATAAATCTGGAGCCGGTGAGCGTGGGTCTCGCGGTATCATTGCAGCACTGGGGCCAGATGGTAAGCCCTCCCGTATCGTAG
TTATCTACACGACGGGGAGTCAGGCAACTATGGATGAACGAAATAGACAGATCGCTGAGATAGGTGCCTCACTGATTAAGCATTGGTAAC
TGTCAGACCAAGTTTACTCATATATACTTTAGATTGATTTAAAACTTCATTTTTAATTTAAAAGGATCTAGGTGAAGATCCTTTTTGATA
ATCTCATGACCAAAATCCCTTAACGTGAGTTTTCGTTCCACTGAGCGTCAGACCCCGTAGAAAAGATCAAAGGATCTTCTTGAGATCCTT
TTTTTCTGCGCGTAATCTGCTGCTTGCAAACAAAAAAACCACCGCTACCAGCGGTGGTTTGTTTGCCGGATCAAGAGCTACCAACTCTTT
TTCCGAAGGTAACTGGCTTCAGCAGAGCGCAGATACCAAATACTGTTCTTCTAGTGTAGCCGTAGTTAGGCCACCACTTCAAGAACTCTG
TAGCACCGCCTACATACCTCGCTCTGCTAATCCTGTTACCAGTGGCTGCTGCCAGTGGCGATAAGTCGTGTCTTACCGGGTTGGACTCAA
GACGATAGTTACCGGATAAGGCGCAGCGGTCGGGCTGAACGGGGGGTTCGTGCACACAGCCCAGCTTGGAGCGAACGACCTACACCGAAC
TGAGATACCTACAGCGTGAGCTATGAGAAAGCGCCACGCTTCCCGAAGGGAGAAAGGCGGACAGGTATCCGGTAAGCGGCAGGGTCGGAA
CAGGAGAGCGCACGAGGGAGCTTCCAGGGGGAAACGCCTGGTATCTTTATAGTCCTGTCGGGTTTCGCCACCTCTGACTTGAGCGTCGAT
TTTTGTGATGCTCGTCAGGGGGGCGGAGCCTATGGAAAAACGCCAGCAACGCGGCCTTTTTACGGTTCCTGGCCTTTTGCTGGCCTTTTG
CTCACATGTTCTTTCCTGCGTTATCCCCTGATTCTGTGGATAACCGTATTACCGCCTTTGAGTGAGCTGATACCGCTCGCCGCAGCCGAA
CGACCGAGCGCAGCGAGTCAGTGAGCGAGGAAGCGGAAGAGCGCCCAATACGCAAACCGCCTCTCCCCGCGCGTTGGCCGATTCATTAAT
GCAGCTGGCACGACAGGTTTCCCGACTGGAAAGCGGGCAGTGAGCGCAACGCAATTAATGTGAGTTAGCTCACTCATTAGGCACCCCAGG
CTTTACACTTTATGCTTCCGGCTCGTATGTTGTGTGGAATTGTGAGCGGATAACAATTTCACACAGGAAACAGCTATGACCATGATTACG
CCAAGCTCTCGAGATCTAG
SEQ ID NO: 37: pAV HLP FVIIIV3 kan
AGCGCCCAATACGCAAACCGCCTCTCCCCGCGCGTTGGCCGATTCATTAATGCAGCTGGCACGACAGGTTTCCCGACTGGAAAGCGGGCA
GTGAGCGCAACGCAATTAATGTGAGTTAGCTCACTCATTAGGCACCCCAGGCTTTACACTTTATGCTTCCGGCTCGTATGTTGTGTGGAA
TTGTGAGCGGATAACAATTTCACACAGGAAACAGCTATGACCATGATTACGCCAAGCTTCCCGGGGGGATCTTTGGCCACTCCCTCTCTG
CGCGCTCGCTCGCTCACTGAGGCCGCCCGGGCAAAGCCCGGGCGTCGGGCGACCTTTGGTCGCCCGGCCTCAGTGAGCGAGCGAGCGCGC
AGAGAGGGAGTGGCCAACTCCATCACTAGGGGTTCCGGAGGGGTGGAGTCGTGACGTGAATTACGTCATAGGGTTAGGGAGGTCGTATAC
TGTTTGCTGCTTGCAATGTTTGCCCATTTTAGGGTGGACACAGGACGCTGTGGTTTCTGAGCCAGGGGGCGACTCAGATCCCAGCCAGTG
GACTTAGCCCCTGTTTGCTCCTCCGATAACTGGGGTGACCTTGGTTAATATTCACCAGCAGCCTCCCCCGTTGCCCCTCTGGATCCACTG
CTTAAATACGGACGAGGACAGGGCCCTGTCTCCTCAGCTTCAGGCACCACCACTGACCTGGGACAGTGAATCGCGGCCGCCACCATGCAG
ATTGAGCTGAGCACCTGCTTCTTCCTGTGCCTGCTGAGGTTCTGCTTCTCTGCCACCAGGAGATACTACCTGGGGGCTGTGGAGCTGAGC
TGGGACTACATGCAGTCTGACCTGGGGGAGCTGCCTGTGGATGCCAGGTTCCCCCCCAGAGTGCCCAAGAGCTTCCCCTTCAACACCTCT
GTGGTGTACAAGAAGACCCTGTTTGTGGAGTTCACTGACCACCTGTTCAACATTGCCAAGCCCAGGCCCCCCTGGATGGGCCTGCTGGGC
CCCACCATCCAGGCTGAGGTGTATGACACTGTGGTGATCACCCTGAAGAACATGGCCAGCCACCCTGTGAGCCTGCATGCTGTGGGGGTG
AGCTACTGGAAGGCCTCTGAGGGGGCTGAGTATGATGACCAGACCAGCCAGAGGGAGAAGGAGGATGACAAGGTGTTCCCTGGGGGCAGC
CACACCTATGTGTGGCAGGTGCTGAAGGAGAATGGCCCCATGGCCTCTGACCCCCTGTGCCTGACCTACAGCTACCTGAGCCATGTGGAC
CTGGTGAAGGACCTGAACTCTGGCCTGATTGGGGCCCTGCTGGTGTGCAGGGAGGGCAGCCTGGCCAAGGAGAAGACCCAGACCCTGCAC
AAGTTCATCCTGCTGTTTGCTGTGTTTGATGAGGGCAAGAGCTGGCACTCTGAAACCAAGAACAGCCTGATGCAGGACAGGGATGCTGCC
TCTGCCAGGGCCTGGCCCAAGATGCACACTGTGAATGGCTATGTGAACAGGAGCCTGCCTGGCCTGATTGGCTGCCACAGGAAGTCTGTG
TACTGGCATGTGATTGGCATGGGCACCACCCCTGAGGTGCACAGCATCTTCCTGGAGGGCCACACCTTCCTGGTCAGGAACCACAGGCAG
GCCAGCCTGGAGATCAGCCCCATCACCTTCCTGACTGCCCAGACCCTGCTGATGGACCTGGGCCAGTTCCTGCTGTTCTGCCACATCAGC
AGCCACCAGCATGATGGCATGGAGGCCTATGTGAAGGTGGACAGCTGCCCTGAGGAGCCCCAGCTGAGGATGAAGAACAATGAGGAGGCT
GAGGACTATGATGATGACCTGACTGACTCTGAGATGGATGTGGTGAGGTTTGATGATGACAACAGCCCCAGCTTCATCCAGATCAGGTCT
GTGGCCAAGAAGCACCCCAAGACCTGGGTGCACTACATTGCTGCTGAGGAGGAGGACTGGGACTATGCCCCCCTGGTGCTGGCCCCTGAT
GACAGGAGCTACAAGAGCCAGTACCTGAACAATGGCCCCCAGAGGATTGGCAGGAAGTACAAGAAGGTCAGGTTCATGGCCTACACTGAT
GAAACCTTCAAGACCAGGGAGGCCATCCAGCATGAGTCTGGCATCCTGGGCCCCCTGCTGTATGGGGAGGTGGGGGACACCCTGCTGATC
ATCTTCAAGAACCAGGCCAGCAGGCCCTACAACATCTACCCCCATGGCATCACTGATGTGAGGCCCCTGTACAGCAGGAGGCTGCCCAAG
GGGGTGAAGCACCTGAAGGACTTCCCCATCCTGCCTGGGGAGATCTTCAAGTACAAGTGGACTGTGACTGTGGAGGATGGCCCCACCAAG
TCTGACCCCAGGTGCCTGACCAGATACTACAGCAGCTTTGTGAACATGGAGAGGGACCTGGCCTCTGGCCTGATTGGCCCCCTGCTGATC
TGCTACAAGGAGTCTGTGGACCAGAGGGGCAACCAGATCATGTCTGACAAGAGGAATGTGATCCTGTTCTCTGTGTTTGATGAGAACAGG
AGCTGGTACCTGACTGAGAACATCCAGAGGTTCCTGCCCAACCCTGCTGGGGTGCAGCTGGAGGACCCTGAGTTCCAGGCCAGCAACATC
ATGCACAGCATCAATGGCTATGTGTTTGACAGCCTGCAGCTGTCTGTGTGCCTGCATGAGGTGGCCTACTGGTACATCCTGAGCATTGGG
GCCCAGACTGACTTCCTGTCTGTGTTCTTCTCTGGCTACACCTTCAAGCACAAGATGGTGTATGAGGACACCCTGACCCTGTTCCCCTTC
TCTGGGGAGACTGTGTTCATGAGCATGGAGAACCCTGGCCTGTGGATTCTGGGCTGCCACAACTCTGACTTCAGGAACAGGGGCATGACT
GCCCTGCTGAAAGTCTCCAGCTGTGACAAGAACACTGGGGACTACTATGAGGACAGCTATGAGGACATCTCTGCCTACCTGCTGAGCAAG
AACAATGCCATTGAGCCCAGGAGCTTCAGCCAGAATGCCACTAATGTGTCTAACAACAGCAACACCAGCAATGACAGCAATGTGTCTCCC
CCAGTGCTGAAGAGGCACCAGAGGGAGATCACCAGGACCACCCTGCAGTCTGACCAGGAGGAGATTGACTATGATGACACCATCTCTGTG
GAGATGAAGAAGGAGGACTTTGACATCTACGACGAGGACGAGAACCAGAGCCCCAGGAGCTTCCAGAAGAAGACCAGGCACTACTTCATT
GCTGCTGTGGAGAGGCTGTGGGACTATGGCATGAGCAGCAGCCCCCATGTGCTGAGGAACAGGGCCCAGTCTGGCTCTGTGCCCCAGTTC
AAGAAGGTGGTGTTCCAGGAGTTCACTGATGGCAGCTTCACCCAGCCCCTGTACAGAGGGGAGCTGAATGAGCACCTGGGCCTGCTGGGC
CCCTACATCAGGGCTGAGGTGGAGGACAACATCATGGTGACCTTCAGGAACCAGGCCAGCAGGCCCTACAGCTTCTACAGCAGCCTGATC
AGCTATGAGGAGGACCAGAGGCAGGGGGCTGAGCCCAGGAAGAACTTTGTGAAGCCCAATGAAACCAAGACCTACTTCTGGAAGGTGCAG
CACCACATGGCCCCCACCAAGGATGAGTTTGACTGCAAGGCCTGGGCCTACTTCTCTGATGTGGACCTGGAGAAGGATGTGCACTCTGGC
CTGATTGGCCCCCTGCTGGTGTGCCACACCAACACCCTGAACCCTGCCCATGGCAGGCAGGTGACTGTGCAGGAGTTTGCCCTGTTCTTC
ACCATCTTTGATGAAACCAAGAGCTGGTACTTCACTGAGAACATGGAGAGGAACTGCAGGGCCCCCTGCAACATCCAGATGGAGGACCCC
ACCTTCAAGGAGAACTACAGGTTCCATGCCATCAATGGCTACATCATGGACACCCTGCCTGGCCTGGTGATGGCCCAGGACCAGAGGATC
AGGTGGTACCTGCTGAGCATGGGCAGCAATGAGAACATCCACAGCATCCACTTCTCTGGCCATGTGTTCACTGTGAGGAAGAAGGAGGAG
TACAAGATGGCCCTGTACAACCTGTACCCTGGGGTGTTTGAGACTGTGGAGATGCTGCCCAGCAAGGCTGGCATCTGGAGGGTGGAGTGC
CTGATTGGGGAGCACCTGCATGCTGGCATGAGCACCCTGTTCCTGGTGTACAGCAACAAGTGCCAGACCCCCCTGGGCATGGCCTCTGGC
CACATCAGGGACTTCCAGATCACTGCCTCTGGCCAGTATGGCCAGTGGGCCCCCAAGCTGGCCAGGCTGCACTACTCTGGCAGCATCAAT
GCCTGGAGCACCAAGGAGCCCTTCAGCTGGATCAAGGTGGACCTGCTGGCCCCCATGATCATCCATGGCATCAAGACCCAGGGGGCCAGG
CAGAAGTTCAGCAGCCTGTACATCAGCCAGTTCATCATCATGTACAGCCTGGATGGCAAGAAGTGGCAGACCTACAGGGGCAACAGCACT
GGCACCCTGATGGTGTTCTTTGGCAATGTGGACAGCTCTGGCATCAAGCACAACATCTTCAACCCCCCCATCATTGCCAGATACATCAGG
CTGCACCCCACCCACTACAGCATCAGGAGCACCCTGAGGATGGAGCTGATGGGCTGTGACCTGAACAGCTGCAGCATGCCCCTGGGCATG
GAGAGCAAGGCCATCTCTGATGCCCAGATCACTGCCAGCAGCTACTTCACCAACATGTTTGCCACCTGGAGCCCCAGCAAGGCCAGGCTG
CACCTGCAGGGCAGGAGCAATGCCTGGAGGCCCCAGGTCAACAACCCCAAGGAGTGGCTGCAGGTGGACTTCCAGAAGACCATGAAGGTG
ACTGGGGTGACCACCCAGGGGGTGAAGAGCCTGCTGACCAGCATGTATGTGAAGGAGTTCCTGATCAGCAGCAGCCAGGATGGCCACCAG
TGGACCCTGTTCTTCCAGAATGGCAAGGTGAAGGTGTTCCAGGGCAACCAGGACAGCTTCACCCCTGTGGTGAACAGCCTGGACCCCCCC
CTGCTGACCAGATACCTGAGGATTCACCCCCAGAGCTGGGTGCACCAGATTGCCCTGAGGATGGAGGTGCTGGGCTGTGAGGCCCAGGAC
CTGTACTGATCGCGAATAAAAGATCTTTATTTTCATTAGATCTGTGTGTTGGTTTTTTGTGTGATGCAGCCCAAGCTGTAGATAAGTAGC
ATGGCGGGTTAATCATTAACTACACCCCTAGTGATGGAGTTGGCCACTCCCTCTCTGCGCGCTCGCTCGCTCACTGAGGCCGCCCGGGCA
AAGCCCGGGCGTCGGGCGACCTTTGGTCGCCCGGCCTCAGTGAGCGAGCGAGCGCGCAGAGAGGGAGTGGCCAAAGATCCGGGCCCGCAT
GCGTCGACAATTCACTGGCCGTCGTTTTACAACGTCGTGACTGGGAAAACCCTGGCGTTACCCAACTTAATCGCCTTGCAGCACATCCCC
CTTTCGCCAGCTGGCGTAATAGCGAAGAGGCCCGCACCGATCGCCCTTCCCAACAGTTGCGCAGCCTGAATGGCGAATGGCATCCATCAC
ACTGGCGGCCGCTCGAGCATGCATCTAGAGGGCCCAATTCGCCCTATAGTGAGTCGTATTACAATTCACTGGCCGTCGTTTTACAACGTC
GTGACTGGGAAAACCCTGGCGTTACCCAACTTAATCGCCTTGCAGCACATCCCCCTTTCGCCAGCTGGCGTAATAGCGAAGAGGCCCGCA
CCGATCGCCCTTCCCAACAGTTGCGCAGCCTGAATGGCGAATGGACGCGCCCTGTAGCGGCGCATTAAGCGCGGCGGGTGTGGTGGTTAC
GCGCAGCGTGACCGCTACACTTGCCAGCGCCCTAGCGCCCGCTCCTTTCGCTTTCTTCCCTTCCTTTCTCGCCACGTTCGCCGGCTTTCC
CCGTCAAGCTCTAAATCGGGGGCTCCCTTTAGGGTTCCGATTTAGTGCTTTACGGCACCTCGACCCCAAAAAACTTGATTAGGGTGATGG
TTCACGTAGTGGGCCATCGCCCTGATAGACGGTTTTTCGCCCTTTGACGTTGGAGTCCACGTTCTTTAATAGTGGACTCTTGTTCCAAAC
TGGAACAACACTCAACCCTATCTCGGTCTATTCTTTTGATTTATAAGGGATTTTGCCGATTTCGGCCTATTGGTTAAAAAATGAGCTGAT
TTAACAAAAATTTAACGCGAATTTTAACAAAATTCAGGGCGCAAGGGCTGCTAAAGGAAGCGGAACACGTAGAAAGCCAGTCCGCAGAAA
CGGTGCTGACCCCGGATGAATGTCAGCTACTGGGCTATCTGGACAAGGGAAAACGCAAGCGCAAAGAGAAAGCAGGTAGCTTGCAGTGGG
CTTACATGGCGATAGCTAGACTGGGCGGTTTTATGGACAGCAAGCGAACCGGAATTGCCAGCTGGGGCGCCCTCTGGTAAGGTTGGGAAG
CCCTGCAAAGTAAACTGGATGGCTTTCTTGCCGCCAAGGATCTGATGGCGCAGGGGATCAAGATCTGATCAAGAGACAGGATGAGGATCG
TTTCGCATGATTGAACAAGATGGATTGCACGCAGGTTCTCCGGCCGCTTGGGTGGAGAGGCTATTCGGCTATGACTGGGCACAACAGACA
ATCGGCTGCTCTGATGCCGCCGTGTTCCGGCTGTCAGCGCAGGGGCGCCCGGTTCTTTTTGTCAAGACCGACCTGTCCGGTGCCCTGAAT
GAACTGCAGGACGAGGCAGCGCGGCTATCGTGGCTGGCCACGACGGGCGTTCCTTGCGCAGCTGTGCTCGACGTTGTCACTGAAGCGGGA
AGGGACTGGCTGCTATTGGGCGAAGTGCCGGGGCAGGATCTCCTGTCATCCCACCTTGCTCCTGCCGAGAAAGTATCCATCATGGCTGAT
GCAATGCGGCGGCTGCATACGCTTGATCCGGCTACCTGCCCATTCGACCACCAAGCGAAACATCGCATCGAGCGAGCACGTACTCGGATG
GAAGCCGGTCTTGTCGATCAGGATGATCTGGACGAAGAGCATCAGGGGCTCGCGCCAGCCGAACTGTTCGCCAGGCTCAAGGCGCGCATG
CCCGACGGCGAGGATCTCGTCGTGACCCATGGCGATGCCTGCTTGCCGAATATCATGGTGGAAAATGGCCGCTTTTCTGGATTCATCGAC
TGTGGCCGGCTGGGTGTGGCGGACCGCTATCAGGACATAGCGTTGGCTACCCGTGATATTGCTGAAGAGCTTGGCGGCGAATGGGCTGAC
CGCTTCCTCGTGCTTTACGGTATCGCCGCTCCCGATTCGCAGCGCATCGCCTTCTATCGCCTTCTTGACGAGTTCTTCTGAATTGAAAAA
GGAAGAGTATGAGTATTCAACATTTCCGTGTCGCCCTTATTCCCTTTTTTGCGGCATTTTGCCTTCCTGTTTTTGCTCACCCAGAAACGC
TGGTGAAAGTAAAAGATGCTGAAGATCAGTTGGGTGCACGAGTGGGTTACATCGAACTGGATCTCAACAGCGGTAAGATCCTTGAGAGTT
TTCGCCCCGAAGAACGTTTTCCAATGATGAGCACTTTTAAAGTTCTGCTATGTGGCGCGGTATTATCCCGTATTGACGCCGGGCAAGAGC
AACTCGGTCGCCGCATACACTATTCTCAGAATGACTTGGTTGAGTACTCACCAGTCACAGAAAAGCATCTTACGGATGGCATGACAGTAA
GAGAATTATGCAGTGCTGCCATAACCATGAGTGATAACACTGCGGCCAACTTACTTCTGACAACGATCGGAGGACCGAAGGAGCTAACCG
CTTTTTTGCACAACATGGGGGATCATGTAACTCGCCTTGATCGTTGGGAACCGGAGCTGAATGAAGCCATACCAAACGACGAGCGTGACA
CCACGATGCCTGTAGCAATGGCAACAACGTTGCGCAAACTATTAACTGGCGAACTACTTACTCTAGCTTCCCGGCAACAATTAATAGACT
GGATGGAGGCGGATAAAGTTGCAGGACCACTTCTGCGCTCGGCCCTTCCGGCTGGCTGGTTTATTGCTGATAAATCTGGAGCCGGTGAGC
GTGGGTCTCGCGGTATCATTGCAGCACTGGGGCCAGATGGTAAGCCCTCCCGTATCGTAGTTATCTACACGACGGGGAGTCAGGCAACTA
TGGATGAACGAAATAGACAGATCGCTGAGATAGGTGCCTCACTGATTAAGCATTGGTAACTGTCAGACCAAGTTTACTCATATATACTTT
AGATTGATTTAAAACTTCATTTTTAATTTAAAAGGATCTAGGTGAAGATCCTTTTTGATAATCTCATGACCAAAATCCCTTAACGTGAGT
TTTCGTTCCACTGAGCGTCAGACCCCGTAGAAAAGATCAAAGGATCTTCTTGAGATCCTTTTTTTCTGCGCGTAATCTGCTGCTTGCAAA
CAAAAAAACCACCGCTACCAGCGGTGGTTTGTTTGCCGGATCAAGAGCTACCAACTCTTTTTCCGAAGGTAACTGGCTTCAGCAGAGCGC
AGATACCAAATACTGTTCTTCTAGTGTAGCCGTAGTTAGGCCACCACTTCAAGAACTCTGTAGCACCGCCTACATACCTCGCTCTGCTAA
TCCTGTTACCAGTGGCTGCTGCCAGTGGCGATAAGTCGTGTCTTACCGGGTTGGACTCAAGACGATAGTTACCGGATAAGGCGCAGCGGT
CGGGCTGAACGGGGGGTTCGTGCACACAGCCCAGCTTGGAGCGAACGACCTACACCGAACTGAGATACCTACAGCGTGAGCTATGAGAAA
GCGCCACGCTTCCCGAAGGGAGAAAGGCGGACAGGTATCCGGTAAGCGGCAGGGTCGGAACAGGAGAGCGCACGAGGGAGCTTCCAGGGG
GAAACGCCTGGTATCTTTATAGTCCTGTCGGGTTTCGCCACCTCTGACTTGAGCGTCGATTTTTGTGATGCTCGTCAGGGGGGCGGAGCC
TATGGAAAAACGCCAGCAACGCGGCCTTTTTACGGTTCCTGGCCTTTTGCTGGCCTTTTGCTCACATGTTCTTTCCTGCGTTATCCCCTG
ATTCTGTGGATAACCGTATTACCGCCTTTGAGTGAGCTGATACCGCTCGCCGCAGCCGAACGACCGAGCGCAGCGAGTCAGTGAGCGAGG
AAGCGGAAG
SEQ ID NO: 38: Hybrid liver-specific promoter (HLP)
TGTTTGCTGCTTGCAATGTTTGCCCATTTTAGGGTGGACACAGGACGCTGTGGTTTCTGAGCCAGGGGGCGACTCAGATCCCAGCCAGTG
GACTTAGCCCCTGTTTGCTCCTCCGATAACTGGGGTGACCTTGGTTAATATTCACCAGCAGCCTCCCCCGTTGCCCCTCTGGATCCACTG
CTTAAATACGGACGAGGACAGGGCCCTGTCTCCTCAGCTTCAGGCACCACCACTGACCTGGGACAGTGAATC
SEQ ID NO: 39: Liver promoter/enhancer 1 (LP1)
CCCTAAAATGGGCAAACATTGCAAGCAGCAAACAGCAAACACACAGCCCTCCCTGCCTGCTGACCTTGGAGCTGGGGCAGAGGTCAGAGA
CCTCTCTGGGCCCATGCCACCTCCAACATCCACTCGACCCCTTGGAATTTCGGTGGAGAGGAGCAGAGGTTGTCCTGGCGTGGTTTAGGT
AGTGTGAGAGGGGAATGACTCCTTTCGGTAAGTGCAGTGGAAGCTGTACACTGCCCAGGCAAAGCGTCCGGGCAGCGTAGGCGGGCGACT
CAGATCCCAGCCAGTGGACTTAGCCCCTGTTTGCTCCTCCGATAACTGGGGTGACCTTGGTTAATATTCACCAGCAGCCTCCCCCGTTGC
CCCTCTGGATCCACTGCTTAAATACGGACGAGGACAGGGCCCTGTCTCCTCAGCTTCAGGCACCACCACTGACCTGGGACAGTGAATCCG
GACTCTAAGGTAAATATAAAATTTTTAAGTGTATAATGTGTTAAACTACTGATTCTAATTGTTTCTCTCTTTTAGATTCCAACCTTTGGA
ACTGA
SEQ ID NO: 40: tTH = truncated Tyrosine Hydroxylase
MSPAGPKVPWFPRKVSELDKCHHLVTKFDPDLDLDHPGFSDQVYRQRRKLIAEIAFQYRHGDPIPRVEYTAEEIATWKEVYTTLKGLYAT
HACGEHLEAFALLERFSGYREDNIPQLEDVSRFLKERTGFQLRPVAGLLSARDFLASLAFRVFQCTQYIRHASSPMHSPEPDCCHELLGH
VPMLADRTFAQFSQDIGLASLGASDEEIEKLSTLYWFTVEFGLCKQNGEVKAYGAGLLSSYGELLHCLSEEPEIRAFDPEAAAVQPYQDQ
TYQSVYFVSESFSDAKDKLRSYASRIQRPFSVKFDPYTLAIDVLDSPQAVRRSLEGVQDELDTLAHALSAIG
SEQ ID NO: 41: PTPS = 6-pyruvoyltetrahydropterin synthase
>ENA|BAA04959|BAA04959.1 Homo sapiens (human) 6-pyruvoyl-tetrahydropterin synthase
ATGAGCACGGAAGGTGGTGGCCGTCGCTGCCAGGCACAAGTGTCCCGCCGCATCTCCTTCAGCGCGAGCCACCGATTGTACAGTAAATTT
CTAAGTGATGAAGAAAACTTGAAACTGTTTGGGAAATGCAACAATCCAAATGGCCATGGGCACAATTATAAAGTTGTGGTGACAGTACAT
GGAGAGATTGACCCTGCTACGGGAATGGTTATGAATCTGGCTGATCTCAAAAAATATATGGAGGAGGCGATTATGCAGCCCCTTGATCAT
AAGAATCTGGATATGGATGTGCCATACTTTGCAGATGTGGTGAGCACGACTGAAAATGTAGCTGTTTATATCTGGGACAACCTCCAGAAA
GTTCTTCCTGTAGGAGTTCTTTATAAAGTAAAAGTATACGAAACTGACAATAATATTGTGGTTTATAAAGGAGAATAG
SEQ ID NO: 42: primer AA16
ccaagctagcATGGAGAAGGGCCCTGTG
SEQ ID NO: 43: primer AA17
ccaagctagcGGTCGACTAAAAAACCTCC
SEQ ID NO: 44: primer AA33
CCAAgctagcATGAGCCCCGCGGGGCCCAAG
SEQ ID NO: 45: primer AA34
CCAAgctagcGGGGGATCTTCGATGCTAGAC
SEQ ID NO: 46: primer AA43
CCAATGGCCAACTCCATCACTAGGGGTTCCTTCTAGATGTTTGCTGCTTGCAATGTTTGC
SEQ ID NO: 47: primer AA44
CCAAGAATTCGCTAGCGATTCACTGTCCCAGGTCAGTG
SEQ ID NO: 48: primer AA57
CCAAGCTAGCTGTTTGCTGCTTGCAATGTTTGC
SEQ ID NO: 49: primer AA67
GATCCTTGCTACGAGCTTGAATGATTCACTGTCCCAGGTCAGT
SEQ ID NO: 50: primer AA68
ACTGACCTGGGACAGTGAATCATTCAAGCTCGTAGCAAGGATC
SEQ ID NO: 51: primer RmuscTHext2
AAAgctagcTTCGATGCTAGACGATCCAG
SEQ ID NO: 52: MLF003noefgp
GCGATCGCGGCTCCCGACATCTTGGACCATTAGCTCCACAGGTATCTTCTTCCCTCTAGTGGTCATAACAGCAGCTTCAGCTACCTCTCA
ATTCAAAAAACCCCTCAAGACCCGTTTAGAGGCCCCAAGGGGTTATGCTATCAATCGTTGCGTTACACACACAAAAAACCAACACACATC
CATCTTCGATGGATAGCGATTTTATTATCTAACTGCTGATCGAGTGTAGCCAGATCTAGTAATCAATTACGGGGTCATTAGTTCATAGCC
CATATATGGAGTTCCGCGTTACATAACTTACGGTAAATGGCCCGCCTGGCTGACCGCCCAACGACCCCCGCCCATTGACGTCAATAATGA
CGTATGTTCCCATAGTAACGCCAATAGGGACTTTCCATTGACGTCAATGGGTGGAGTATTTACGGTAAACTGCCCACTTGGCAGTACATC
AAGTGTATCATATGCCAAGTACGCCCCCTATTGACGTCAATGACGGTAAATGGCCCGCCTGGCATTATGCCCAGTACATGACCTTATGGG
ACTTTCCTACTTGGCAGTACATCTACGTATTAGTCATCGCTATTACCATGCTGATGCGGTTTTGGCAGTACATCAATGGGCGTGGATAGC
GGTTTGACTCACGGGGATTTCCAAGTCTCCACCCCATTGACGTCAATGGGAGTTTGTTTTGGCACCAAAATCAACGGGACTTTCCAAAAT
GTCGTAACAACTCCGCCCCATTGACGCAAATGGGCGGTAGGCGTGTACGGTGGGAGGTCTATATAAGCAGAGCTGGTTTAGTGAACCGTC
AGATCAGATCTTTGTCGATCCTACCATCCACTCGACACACCCGCCAGCTAGAGATCCCGGGACCATGAGCCCCGCGGGGCCCAAGGTCCC
CTGGTTCCCAAGAAAAGTGTCAGAGCTGGACAAGTGTCATCACCTGGTCACCAAGTTCGACCCTGACCTGGACTTGGACCACCCGGGCTT
CTCGGACCAGGTGTACCGCCAGCGCAGGAAGCTGATTGCTGAGATCGCCTTCCAGTACAGGCACGGCGACCCGATTCCCCGTGTGGAGTA
CACCGCCGAGGAGATTGCCACCTGGAAGGAGGTCTACACCACGCTGAAGGGCCTCTACGCCACGCACGCCTGCGGGGAGCACCTGGAGGC
CTTTGCTTTGCTGGAGCGCTTCAGCGGCTACCGGGAAGACAATATCCCCCAGCTGGAGGACGTCTCCCGCTTCCTGAAGGAGCGCACGGG
CTTCCAGCTGCGGCCTGTGGCCGGCCTGCTGTCCGCCCGGGACTTCCTGGCCAGCCTGGCCTTCCGCGTGTTCCAGTGCACCCAGTATAT
CCGCCACGCGTCCTCGCCCATGCACTCCCCTGAGCCGGACTGCTGCCACGAGCTGCTGGGGCACGTGCCCATGCTGGCCGACCGCACCTT
CGCGCAGTTCTCGCAGGACATTGGCCTGGCGTCCCTGGGGGCCTCGGATGAGGAAATTGAGAAGCTGTCCACGCTGTACTGGTTCACGGT
GGAGTTCGGGCTGTGTAAGCAGAACGGGGAGGTGAAGGCCTATGGTGCCGGGCTGCTGTCCTCCTACGGGGAGCTCCTGCACTGCCTGTC
TGAGGAGCCTGAGATTCGGGCCTTCGACCCTGAGGCTGCGGCCGTGCAGCCCTACCAAGACCAGACGTACCAGTCAGTCTACTTCGTGTC
TGAGAGCTTCAGTGACGCCAAGGACAAGCTCAGGAGCTATGCCTCACGCATCCAGCGCCCCTTCTCCGTGAAGTTCGACCCGTACACGCT
GGCCATCGACGTGCTGGACAGCCCCCAGGCCGTGCGGCGCTCCCTGGAGGGTGTCCAGGATGAGCTGGACACCCTTGCCCATGCGCTGAG
TGCCATTGGCTAAGACGCCACCTAATCAACCTCTGGATTACAAAATTTGTGAAAGATTGACTGGTATTCTTAACTATGTTGCTCCTTTTA
CGCTATGTGGATACGCTGCTTTAATGCCTTTGTATCATGCTATTGCTTCCCGTATGGCTTTCATTTTCTCCTCCTTGTATAAATCCTGGT
TGCTGTCTCTTTATGAGGAGTTGTGGCCCGTTGTCAGGCAACGTGGCGTGGTGTGCACTGTGTTTGCTGACGCAACCCCCACTGGTTGGG
GCATTGCCACCACCTGTCAGCTCCTTTCCGGGACTTTCGCTTTCCCCCTCCCTATTGCCACGGCGGAACTCATCGCCGCCTGCCTTGCCC
GCTGCTGGACAGGGGCTCGGCTGTTGGGCACTGACAATTCCGTGGTGTTGTCGGGGAAATCATCGTCCTTTCCCATCTTGACTGACTGAG
ATACAGCGTACCTTCAGCTCACAGACATGATAAGATACATTGATGAGTTTGGACAAACCACAACTAGAATGCAGTGAAAAAAATGCTTTA
TTTGTGAAATTTGTGATGCTATTGCTTTATTTGTAACCATTATAAGCTGCAATAAACAAGTTAACAACAACAATTGCATTCATTTTATGT
TTCAGGTTCAGGGGGAGGTGTGGGAGGTTTTTTAAAGCAAGTAAAACCTCTACAAATGTGGTATTGGCCCATCTCTATCGGTATCGTAGC
ATAACCCCTTGGGGCCTCTAAACGGGTCTTGAGGGGTTTTTTGTGCCCCTCGGGCCGGATTGCTATCTACCGGCATTGGCGCAGAAAAAA
ATGCCTGATGCGACGCTGCGCGTCTTATACTCCCACATATGCCAGATTCAGCAACGGATACGGCTTCCCCAACTTGCCCACTTCCATACG
TGTCCTCCTTACCAGAAATTTATCCTTAAGGTCGTCAGCTATCCTGCAGGCGATCTCTCGATTTCGATCAAGACATTCCTTTAATGGTCT
TTTCTGGACACCACTAGGGGTCAGAAGTAGTTCATCAAACTTTCTTCCCTCCCTAATCTCATTGGTTACCTTGGGCTATCGAAACTTAAT
TAACCAGTCAAGTCAGCTACTTGGCGAGATCGACTTGTCTGGGTTTCGACTACGCTCAGAATTGCGTCAGTCAAGTTCGATCTGGTCCTT
GCTATTGCACCCGTTCTCCGATTACGAGTTTCATTTAAATCATGTGAGCAAAAGGCCAGCAAAAGGCCAGGAACCGTAAAAAGGCCGCGT
TGCTGGCGTTTTTCCATAGGCTCCGCCCCCCTGACGAGCATCACAAAAATCGACGCTCAAGTCAGAGGTGGCGAAACCCGACAGGACTAT
AAAGATACCAGGCGTTTCCCCCTGGAAGCTCCCTCGTGCGCTCTCCTGTTCCGACCCTGCCGCTTACCGGATACCTGTCCGCCTTTCTCC
CTTCGGGAAGCGTGGCGCTTTCTCATAGCTCACGCTGTAGGTATCTCAGTTCGGTGTAGGTCGTTCGCTCCAAGCTGGGCTGTGTGCACG
AACCCCCCGTTCAGCCCGACCGCTGCGCCTTATCCGGTAACTATCGTCTTGAGTCCAACCCGGTAAGACACGACTTATCGCCACTGGCAG
CAGCCACTGGTAACAGGATTAGCAGAGCGAGGTATGTAGGCGGTGCTACAGAGTTCTTGAAGTGGTGGCCTAACTACGGCTACACTAGAA
GAACAGTATTTGGTATCTGCGCTCTGCTGAAGCCAGTTACCTTCGGAAAAAGAGTTGGTAGCTCTTGATCCGGCAAACAAACCACCGCTG
GTAGCGGTGGTTTTTTTGTTTGCAAGCAGCAGATTACGCGCAGAAAAAAAGGATCTCAAGAAGATCCTTTGATCTTTTCTACGGGGTCTG
ACGCTCAGTGGAACGAAAACTCACGTTAAGGGATTTTGGTCATGAGATTATCAAAAAGGATCTTCACCTAGATCCTTTTAAATTAAAAAT
GAAGTTTTAAATCAATCTAAAGTATATATGAGTAAACTTGGTCTGACAGTTACCAATGCTTAATCAGTGAGGCACCTATCTCAGCGATCT
GTCTATTTCGTTCATCCATAGTTGCATTTAAATTTCCGAACTCTCCAAGGCCCTCGTCGGAAAATCTTCAAACCTTTCGTCCGATCCATC
TTGCAGGCTACCTCTCGAACGAACTATCGCAAGTCTCTTGGCCGGCCTTGCGCCTTGGCTATTGCTTGGCAGCGCCTATCGCCAGGTATT
ACTCCAATCCCGAATATCCGAGATCGGGATCACCCGAGAGAAGTTCAACCTACATCCTCAATCCCGATCTATCCGAGATCCGAGGAATAT
CGAAATCGGGGCGCGCCTGGTGTACCGAGAACGATCCTCTCAGTGCGAGTCTCGACGATCCATATCGTTGCTTGGCAGTCAGCCAGTCGG
AATCCAGCTTGGGACCCAGGAAGTCCAATCGTCAGATATTGTACTCAAGCCTGGTCACGGCAGCGTACCGATCTGTTTAAACCTAGATAT
TGATAGTCTGATCGGTCAACGTATAATCGAGTCCTAGCTTTTGCAAACATCTATCAAGAGACAGGATCAGCAGGAGGCTTTCGCATGAGT
ATTCAACATTTCCGTGTCGCCCTTATTCCCTTTTTTGCGGCATTTTGCCTTCCTGTTTTTGCTCACCCAGAAACGCTGGTGAAAGTAAAA
GATGCTGAAGATCAGTTGGGTGCGCGAGTGGGTTACATCGAACTGGATCTCAACAGCGGTAAGATCCTTGAGAGTTTTCGCCCCGAAGAA
CGCTTTCCAATGATGAGCACTTTTAAAGTTCTGCTATGTGGCGCGGTATTATCCCGTATTGACGCCGGGCAAGAGCAACTCGGTCGCCGC
ATACACTATTCTCAGAATGACTTGGTTGAGTATTCACCAGTCACAGAAAAGCATCTTACGGATGGCATGACAGTAAGAGAATTATGCAGT
GCTGCCATAACCATGAGTGATAACACTGCGGCCAACTTACTTCTGACAACGATTGGAGGACCGAAGGAGCTAACCGCTTTTTTGCACAAC
ATGGGGGATCATGTAACTCGCCTTGATCGTTGGGAACCGGAGCTGAATGAAGCCATACCAAACGACGAGCGTGACACCACGATGCCTGTA
GCAATGGCAACAACCTTGCGTAAACTATTAACTGGCGAACTACTTACTCTAGCTTCCCGGCAACAGTTGATAGACTGGATGGAGGCGGAT
AAAGTTGCAGGACCACTTCTGCGCTCGGCCCTTCCGGCTGGCTGGTTTATTGCTGATAAATCTGGAGCCGGTGAGCGTGGGTCTCGCGGT
ATCATTGCAGCACTGGGGCCAGATGGTAAGCCCTCCCGTATCGTAGTTATCTACACGACGGGGAGTCAGGCAACTATGGATGAACGAAAT
AGACAGATCGCTGAGATAGGTGCCTCACTGATTAAGCATTGGTAACCGATTCTAGGTGCATTGGCGCAGAAAAAAATGCCTGATGCGACG
CTGCGCGTCTTATACTCCCACATATGCCAGATTCAGCAACGGATACGGCTTCCCCAACTTGCCCACTTCCATACGTGTCCTCCTTACCAG
AAATTTATCCTTAAGATCCCGAATCGTTTAAACTCGACTCTGGCTCTATCGAATCTCCGTCGTTTCGAGCTTACGCGAACAGCCGTGGCG
CTCATTTGCTCGTCGGGCATCGAATCTCGTCAGCTATCGTCAGCTTACCTTTTTGGCA
SEQ ID NO: 53 MDL004
GCGATCGCGGCTCCCGACATCTTGGACCATTAGCTCCACAGGTATCTTCTTCCCTCTAGTGGTCATAACAGCAGCTTCAGCTACCTCTCA
ATTCAAAAAACCCCTCAAGACCCGTTTAGAGGCCCCAAGGGGTTATGCTATCAATCGTTGCGTTACACACACAAAAAACCAACACACATC
CATCTTCGATGGATAGCGATTTTATTATCTAACTGCTGATCGAGTGTAGCCAGATCTAGTAATCAATTACGGGGTCATTAGTTCATAGCC
CATATATGGAGTTCCGCGTTACATAACTTACGGTAAATGGCCCGCCTGGCTGACCGCCCAACGACCCCCGCCCATTGACGTCAATAATGA
CGTATGTTCCCATAGTAACGCCAATAGGGACTTTCCATTGACGTCAATGGGTGGAGTATTTACGGTAAACTGCCCACTTGGCAGTACATC
AAGTGTATCATATGCCAAGTACGCCCCCTATTGACGTCAATGACGGTAAATGGCCCGCCTGGCATTATGCCCAGTACATGACCTTATGGG
ACTTTCCTACTTGGCAGTACATCTACGTATTAGTCATCGCTATTACCATGCTGATGCGGTTTTGGCAGTACATCAATGGGCGTGGATAGC
GGTTTGACTCACGGGGATTTCCAAGTCTCCACCCCATTGACGTCAATGGGAGTTTGTTTTGGCACCAAAATCAACGGGACTTTCCAAAAT
GTCGTAACAACTCCGCCCCATTGACGCAAATGGGCGGTAGGCGTGTACGGTGGGAGGTCTATATAAGCAGAGCTGGTTTAGTGAACCGTC
AGATCAGATCTTTGTCGATCCTACCATCCACTCGACACACCCGCCAGCAATATGGCCACAACCGCGGCCGTAGATCCCGGGACCATGGAG
AAGCCGCGGGGAGTCAGGTGCACCAATGGGTTCTCCGAGCGGGAGCTGCCGCGGCCCGGGGCCAGCCCGCCTGCCGAGAAGTCCCGGCCG
CCCGAGGCCAAGGGCGCACAGCCGGCCGACGCCTGGAAGGCAGGGCGGCACCGCAGCGAGGAGGAAAACCAGGTGAACCTCCCCAAACTG
GCGGCTGCTTACTCGTCCATTCTGCTCTCGCTGGGCGAGGACCCCCAGCGGCAGGGGCTGCTCAAGACGCCCTGGAGGGCGGCCACCGCC
ATGCAGTACTTCACCAAGGGATACCAGGAGACCATCTCAGATGTCCTGAATGATGCTATATTTGATGAAGATCATGACGAGATGGTGATT
GTGAAGGACATAGATATGTTCTCCATGTGTGAGCATCACCTTGTTCCATTTGTAGGAAGGGTCCATATTGGCTATCTTCCTAACAAGCAA
GTCCTTGGTCTCAGTAAACTTGCCAGGATTGTAGAAATCTACAGTAGACGACTACAAGTTCAAGAGCGCCTCACCAAACAGATTGCGGTG
GCCATCACAGAAGCCTTGCAGCCTGCTGGCGTTGGAGTAGTGATTGAAGCGACACACATGTGCATGGTAATGCGAGGCGTGCAGAAAATG
AACAGCAAGACTGTCACTAGCACCATGCTGGGCGTGTTCCGGGAAGACCCCAAGACTCGGGAGGAGTTCCTCACACTAATCAGGAGCTGA
GACTATAGGGTGGGTATTATGTGTTCATCAACCATCCTAAAAATACCCGGTAAACAGGTGCAGCCCCAGATCTGGGCAGCAGGAGGGGGC
AGTGGGAAGCTTAACGCGCCACGACTATAGGGTGGGTATTATGTGTTCATCAACCATCCTAAAAATACCCGGTAAACAGGTGCAGCCCCA
GATCTGGGCAGCAGGAGGGGGCAGTGGGAAGCTTATCTAGTCTCGAGGTACCGAGCTCTTACGCGTGCTAGCTCGAGATCTGGATATCGA
CTATAGGGTGGGTATTATGTGTTCATCAACCATCCTAAAAATACCCGGTAAACAGGTGCAGCCCCAGATCTGGGCAGCAGGAGGGGGCAG
TGGGTCTGTTCTATTTTTACCAGCCAGTTGCTGCTGGACACAGTTTTCATAGCCTCCCCTCGGCTCTGCCCCTCACAGTCTGCAGTCTAC
GGCGAGGCACAGGCCAGCCCAGCTCCACGAGGACTGAACAAGAAGCTTGATATCGAATTGGTACCATCGAGGAACTGAAAAACCAGAAAG
TTAACTGGTAAGTTTAGTCTTTTTGTCTTTTATTTCAGGTCCCGGATCCGGTGGTGGTGCAAATCAAAGAACTGCTCCTCAGTGGATATC
GCCTTTACTTCTAGGCCACCATGAGCGCGGCGGGTGACCTTCGTCGCCGCGCGCGACTGTCGCGCCTCGTGTCCTTCAGCGCGAGCCACC
GGCTGCACAGCCCATCTCTGAGCGATGAAGAGAACTTAAGAGTGTTTGGGAAATGCAACAATCCGAATGGCCACGGGCACAACTATAAAG
TTGTGGTGACAGTCCATGGAGAGATTGATCCTGTTACAGGAATGGTTATGAATTTGACCGACCTCAAAGAATACATGGAGGAGGCCATCA
TGAAGCCTCTTGATCACAAGAACCTGGACCTGGATGTGCCGTACTTTGCGGATGCTGTGAGCACGACAGAAAATGTAGCTGTCTACATCT
GGGAAAGCCTCCAGAAACTTCTTCCAGTGGGAGCTCTTTATAAAGTAAAAGTGTTTGAAACCGACAACAACATCGTAGTCTATAAAGGAG
AATAGTAATCAACCTCTGGATTACAAAATTTGTGAAAGATTGACTGGTATTCTTAACTATGTTGCTCCTTTTACGCTATGTGGATACGCT
GCTTTAATGCCTTTGTATCATGCTATTGCTTCCCGTATGGCTTTCATTTTCTCCTCCTTGTATAAATCCTGGTTGCTGTCTCTTTATGAG
GAGTTGTGGCCCGTTGTCAGGCAACGTGGCGTGGTGTGCACTGTGTTTGCTGACGCAACCCCCACTGGTTGGGGCATTGCCACCACCTGT
CAGCTCCTTTCCGGGACTTTCGCTTTCCCCCTCCCTATTGCCACGGCGGAACTCATCGCCGCCTGCCTTGCCCGCTGCTGGACAGGGGCT
CGGCTGTTGGGCACTGACAATTCCGTGGTGTTGTCGGGGAAATCATCGTCCTTTCCCATCTTGACTGACTGAGATACAGCGTACCTTCAG
CTCACAGACATGATAAGATACATTGATGAGTTTGGACAAACCACAACTAGAATGCAGTGAAAAAAATGCTTTATTTGTGAAATTTGTGAT
GCTATTGCTTTATTTGTAACCATTATAAGCTGCAATAAACAAGTTAACAACAACAATTGCATTCATTTTATGTTTCAGGTTCAGGGGGAG
GTGTGGGAGGTTTTTTAAAGCAAGTAAAACCTCTACAAATGTGGTATTGGCCCATCTCTATCGGTATCGTAGCATAACCCCTTGGGGCCT
CTAAACGGGTCTTGAGGGGTTTTTTGTGCCCCTCGGGCCGGATTGCTATCTACCGGCATTGGCGCAGAAAAAAATGCCTGATGCGACGCT
GCGCGTCTTATACTCCCACATATGCCAGATTCAGCAACGGATACGGCTTCCCCAACTTGCCCACTTCCATACGTGTCCTCCTTACCAGAA
ATTTATCCTTAAGGTCGTCAGCTATCCTGCAGGCGATCTCTCGATTTCGATCAAGACATTCCTTTAATGGTCTTTTCTGGACACCACTAG
GGGTCAGAAGTAGTTCATCAAACTTTCTTCCCTCCCTAATCTCATTGGTTACCTTGGGCTATCGAAACTTAATTAACCAGTCAAGTCAGC
TACTTGGCGAGATCGACTTGTCTGGGTTTCGACTACGCTCAGAATTGCGTCAGTCAAGTTCGATCTGGTCCTTGCTATTGCACCCGTTCT
CCGATTACGAGTTTCATTTAAATCATGTGAGCAAAAGGCCAGCAAAAGGCCAGGAACCGTAAAAAGGCCGCGTTGCTGGCGTTTTTCCAT
AGGCTCCGCCCCCCTGACGAGCATCACAAAAATCGACGCTCAAGTCAGAGGTGGCGAAACCCGACAGGACTATAAAGATACCAGGCGTTT
CCCCCTGGAAGCTCCCTCGTGCGCTCTCCTGTTCCGACCCTGCCGCTTACCGGATACCTGTCCGCCTTTCTCCCTTCGGGAAGCGTGGCG
CTTTCTCATAGCTCACGCTGTAGGTATCTCAGTTCGGTGTAGGTCGTTCGCTCCAAGCTGGGCTGTGTGCACGAACCCCCCGTTCAGCCC
GACCGCTGCGCCTTATCCGGTAACTATCGTCTTGAGTCCAACCCGGTAAGACACGACTTATCGCCACTGGCAGCAGCCACTGGTAACAGG
ATTAGCAGAGCGAGGTATGTAGGCGGTGCTACAGAGTTCTTGAAGTGGTGGCCTAACTACGGCTACACTAGAAGAACAGTATTTGGTATC
TGCGCTCTGCTGAAGCCAGTTACCTTCGGAAAAAGAGTTGGTAGCTCTTGATCCGGCAAACAAACCACCGCTGGTAGCGGTGGTTTTTTT
GTTTGCAAGCAGCAGATTACGCGCAGAAAAAAAGGATCTCAAGAAGATCCTTTGATCTTTTCTACGGGGTCTGACGCTCAGTGGAACGAA
AACTCACGTTAAGGGATTTTGGTCATGAGATTATCAAAAAGGATCTTCACCTAGATCCTTTTAAATTAAAAATGAAGTTTTAAATCAATC
TAAAGTATATATGAGTAAACTTGGTCTGACAGTTACCAATGCTTAATCAGTGAGGCACCTATCTCAGCGATCTGTCTATTTCGTTCATCC
ATAGTTGCATTTAAATTTCCGAACTCTCCAAGGCCCTCGTCGGAAAATCTTCAAACCTTTCGTCCGATCCATCTTGCAGGCTACCTCTCG
AACGAACTATCGCAAGTCTCTTGGCCGGCCTTGCGCCTTGGCTATTGCTTGGCAGCGCCTATCGCCAGGTATTACTCCAATCCCGAATAT
CCGAGATCGGGATCACCCGAGAGAAGTTCAACCTACATCCTCAATCCCGATCTATCCGAGATCCGAGGAATATCGAAATCGGGGCGCGCC
TGGTGTACCGAGAACGATCCTCTCAGTGCGAGTCTCGACGATCCATATCGTTGCTTGGCAGTCAGCCAGTCGGAATCCAGCTTGGGACCC
AGGAAGTCCAATCGTCAGATATTGTACTCAAGCCTGGTCACGGCAGCGTACCGATCTGTTTAAACCTAGATATTGATAGTCTGATCGGTC
AACGTATAATCGAGTCCTAGCTTTTGCAAACATCTATCAAGAGACAGGATCAGCAGGAGGCTTTCGCATGAGTATTCAACATTTCCGTGT
CGCCCTTATTCCCTTTTTTGCGGCATTTTGCCTTCCTGTTTTTGCTCACCCAGAAACGCTGGTGAAAGTAAAAGATGCTGAAGATCAGTT
GGGTGCGCGAGTGGGTTACATCGAACTGGATCTCAACAGCGGTAAGATCCTTGAGAGTTTTCGCCCCGAAGAACGCTTTCCAATGATGAG
CACTTTTAAAGTTCTGCTATGTGGCGCGGTATTATCCCGTATTGACGCCGGGCAAGAGCAACTCGGTCGCCGCATACACTATTCTCAGAA
TGACTTGGTTGAGTATTCACCAGTCACAGAAAAGCATCTTACGGATGGCATGACAGTAAGAGAATTATGCAGTGCTGCCATAACCATGAG
TGATAACACTGCGGCCAACTTACTTCTGACAACGATTGGAGGACCGAAGGAGCTAACCGCTTTTTTGCACAACATGGGGGATCATGTAAC
TCGCCTTGATCGTTGGGAACCGGAGCTGAATGAAGCCATACCAAACGACGAGCGTGACACCACGATGCCTGTAGCAATGGCAACAACCTT
GCGTAAACTATTAACTGGCGAACTACTTACTCTAGCTTCCCGGCAACAGTTGATAGACTGGATGGAGGCGGATAAAGTTGCAGGACCACT
TCTGCGCTCGGCCCTTCCGGCTGGCTGGTTTATTGCTGATAAATCTGGAGCCGGTGAGCGTGGGTCTCGCGGTATCATTGCAGCACTGGG
GCCAGATGGTAAGCCCTCCCGTATCGTAGTTATCTACACGACGGGGAGTCAGGCAACTATGGATGAACGAAATAGACAGATCGCTGAGAT
AGGTGCCTCACTGATTAAGCATTGGTAACCGATTCTAGGTGCATTGGCGCAGAAAAAAATGCCTGATGCGACGCTGCGCGTCTTATACTC
CCACATATGCCAGATTCAGCAACGGATACGGCTTCCCCAACTTGCCCACTTCCATACGTGTCCTCCTTACCAGAAATTTATCCTTAAGAT
CCCGAATCGTTTAAACTCGACTCTGGCTCTATCGAATCTCCGTCGTTTCGAGCTTACGCGAACAGCCGTGGCGCTCATTTGCTCGTCGGG
CATCGAATCTCGTCAGCTATCGTCAGCTTACCTTTTTGGCA

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. (canceled)

106. (canceled)

107. (canceled)

108. (canceled)

109. (canceled)

110. (canceled)

111. (canceled)

112. (canceled)

113. (canceled)

114. (canceled)

115. (canceled)

116. (canceled)

117. (canceled)

118. (canceled)

119. (canceled)

120. (canceled)

121. (canceled)

122. (canceled)

123. (canceled)

124. (canceled)

125. (canceled)

126. (canceled)

127. (canceled)

128. (canceled)

129. (canceled)

130. (canceled)

131. (canceled)

132. An expression system comprising:
a first polynucleotide (N1) which upon expression encodes a GTP-cyclohydrolase 1 (GCH1; EC 3.5.4.16) polypeptide or a biologically active fragment or variant thereof, wherein said polynucleotide is operably linked to a first promoter, and wherein the biological activity is enzymatic activity of GCH1; and
a second polynucleotide (N2) which upon expression encodes a tyrosine hydroxylase (TH; EC 1.14.16.2) polypeptide or a biologically active fragment or variant thereof, wherein said polynucleotide is operably linked to a second promoter, and wherein the biological activity is enzymatic activity of TH; and
a third polynucleotide (N3) which upon expression encodes a 6-pyruvoyltetrahydropterin synthase (PTPS, EC 4.2.3.12) polypeptide or a biologically active fragment or variant thereof, wherein said polynucleotide is operably linked to a third promoter, and wherein the biological activity is enzymatic activity of PTPS.

133. The expression system according to claim 132, further comprising a linker between the polynucleotide sequences encoding P1 and P2, and a linker between the polynucleotide sequences encoding P2 and P3, optionally wherein the linker is an Internal Ribosome Entry Site (IRES).

134. The expression system according to claim 132, wherein said expression system comprises a first polynucleotide operably linked to a first promoter, wherein said first polynucleotide upon expression encodes a first, a second and a third polypeptide, wherein said first, second and third polypeptide are independently selected from the group consisting of a GCH1 polypeptide, a TH polypeptide and a PTPS polypeptide or a biologically active fragment or variant thereof.

135. The expression system according to claim 132, wherein the GTP-cyclohydrolase 1 (GCH1) polypeptide is at least 70% identical to a polypeptide selected from the group consisting of SEQ ID NO: 1, SEQ ID NO: 2, SEQ ID NO: 3, SEQ ID NO: 4, SEQ ID NO: 5 and SEQ ID NO: 6, more preferably at least 75% identical to a polypeptide selected from the group consisting of SEQ ID NO: 1, SEQ ID NO: 2, SEQ ID NO: 3, SEQ ID NO: 4, SEQ ID NO: 5 and SEQ ID NO: 6, more preferably at least 80% identical to a polypeptide selected from the group consisting of SEQ ID NO: 1, SEQ ID NO: 2, SEQ ID NO: 3, SEQ ID NO: 4, SEQ ID NO: 5 and SEQ ID NO: 6, more preferably at least 85% identical to a polypeptide selected from the group consisting of SEQ ID NO: 1, SEQ ID NO: 2, SEQ ID NO: 3, SEQ ID NO: 4, SEQ ID NO: 5 and SEQ ID NO: 6, more preferably at least 90% identical to a polypeptide selected from the group consisting of SEQ ID NO: 1, SEQ ID NO: 2, SEQ ID NO: 3, SEQ ID NO: 4, SEQ ID NO: 5 and SEQ ID NO: 6, more preferably at least 95% identical to a polypeptide selected from the group consisting of SEQ ID NO: 1, SEQ ID NO: 2, SEQ ID NO: 3, SEQ ID NO: 4, SEQ ID NO: 5 and SEQ ID NO: 6, more preferably at least 96% identical to a polypeptide selected from the group consisting of SEQ ID NO: 1, SEQ ID NO: 2, SEQ ID NO: 3, SEQ ID NO: 4, SEQ ID NO: 5 and SEQ ID NO: 6, more preferably at least 97% identical to a polypeptide selected from the group consisting of SEQ ID NO: 1, SEQ ID NO: 2, SEQ ID NO: 3, SEQ ID NO: 4, SEQ ID NO: 5 and SEQ ID NO: 6, more preferably at least 98% identical to a polypeptide selected from the group consisting of SEQ ID NO: 1, SEQ ID NO: 2, SEQ ID NO: 3, SEQ ID NO: 4, SEQ ID NO: 5 and SEQ ID NO: 6, more preferably at least 99% identical to a polypeptide selected from the group consisting of SEQ ID NO: 1, SEQ ID NO: 2, SEQ ID NO: 3, SEQ ID NO: 4, SEQ ID NO: 5 and SEQ ID NO: 6, more preferably 100% identical to a polypeptide selected from the group consisting of SEQ ID NO: 1, SEQ ID NO: 2, SEQ ID NO: 3, SEQ ID NO: 4, SEQ ID NO: 5 and SEQ ID NO: 6.

136. The expression system according to claim 132, wherein the tyrosine hydroxylase (TH) polypeptide is at least 70% identical to a polypeptide selected from the group consisting of or SEQ ID NO: 40, SEQ ID NO: 7, SEQ ID NO: 8, SEQ ID NO: 9, SEQ ID NO: 10, SEQ ID NO: 11, SEQ ID NO: 12 SEQ ID NO: 13 and SEQ ID NO: 14, more preferably at least 75% identical to a polypeptide selected from the group consisting of or SEQ ID NO: 40, SEQ ID NO: 7, SEQ ID NO: 8, SEQ ID NO: 9, SEQ ID NO: 10, SEQ ID NO: 11, SEQ ID NO: 12 SEQ ID NO: 13, SEQ ID NO: 14, SEQ ID NO: 15, SEQ ID NO: 16, and SEQ ID NO: 17 more preferably at least 80% identical to a polypeptide selected from the group consisting of SEQ ID NO: 40, SEQ ID NO: 7, SEQ ID NO: 8, SEQ ID NO: 9, SEQ ID NO: 10, SEQ ID NO: 11, SEQ ID NO: 12 SEQ ID NO: 13, SEQ ID NO: 14, SEQ ID NO: 15, SEQ ID NO: 16, and SEQ ID NO: 17 more preferably at least 85% identical to a polypeptide selected from the group consisting of SEQ ID NO: 40, SEQ ID NO: 7, SEQ ID NO: 8, SEQ ID NO: 9, SEQ ID NO: 10, SEQ ID NO: 11, SEQ ID NO: 12 SEQ ID NO: 13, SEQ ID NO: 14, SEQ ID NO: 15, SEQ ID NO: 16, and SEQ ID NO: 17 more preferably at least 90% identical to a polypeptide selected from the group consisting of SEQ ID NO: 40, SEQ ID NO: 7, SEQ ID NO: 8, SEQ ID NO: 9, SEQ ID NO: 10, SEQ ID NO: 11, SEQ ID NO: 12 SEQ ID NO: 13, SEQ ID NO: 14, SEQ ID NO: 15, SEQ ID NO: 16, and SEQ ID NO: 17 more preferably at least 95% identical to a polypeptide selected from the group consisting of SEQ ID NO: 40, SEQ ID NO: 7, SEQ ID NO: 8, SEQ ID NO: 9, SEQ ID NO: 10, SEQ ID NO: 11, SEQ ID NO: 12 SEQ ID NO: 13, SEQ ID NO: 14, SEQ ID NO: 15, SEQ ID NO: 16, and SEQ ID NO: 17 more preferably at least 96% identical to a polypeptide selected from the group consisting of SEQ ID NO: 40, SEQ ID NO: 7, SEQ ID NO: 8, SEQ ID NO: 9, SEQ ID NO: 10, SEQ ID NO: 11, SEQ ID NO: 12 SEQ ID NO: 13, SEQ ID NO: 14, SEQ ID NO: 15, SEQ ID NO: 16, and SEQ ID NO: 17 more preferably at least 97% identical to a polypeptide selected from the group consisting of SEQ ID NO: 40, SEQ ID NO: 7, SEQ ID NO: 8, SEQ ID NO: 9, SEQ ID NO: 10, SEQ ID NO: 11, SEQ ID NO: 12 SEQ ID NO: 13, SEQ ID NO: 14, SEQ ID NO: 15, SEQ ID NO: 16, and SEQ ID NO: 17 more preferably at least 98% identical to a polypeptide selected from the group consisting of SEQ ID NO: 40, SEQ ID NO: 7, SEQ ID NO: 8, SEQ ID NO: 9, SEQ ID NO: 10, SEQ ID NO: 11, SEQ ID NO: 12 SEQ ID NO: 13, SEQ ID NO: 14, SEQ ID NO: 15, SEQ ID NO: 16, and SEQ ID NO: 17 more preferably at least 99% identical to a polypeptide selected from the group consisting of SEQ ID NO: 40, SEQ ID NO: 7, SEQ ID NO: 8, SEQ ID NO: 9, SEQ ID NO: 10, SEQ ID NO: 11, SEQ ID NO: 12 SEQ ID NO: 13, SEQ ID NO: 14, SEQ ID NO: 15, SEQ ID NO: 16, and SEQ ID NO: 17 more preferably 100% identical to a polypeptide selected from the group consisting of SEQ ID NO: 40, SEQ ID NO: 7, SEQ ID NO: 8, SEQ ID NO: 9, SEQ ID NO: 10, SEQ ID NO: 11, SEQ ID NO: 12 SEQ ID NO: 13, SEQ ID NO: 14, SEQ ID NO: 15, SEQ ID NO: 16 and SEQ ID NO: 17.

137. The expression system according to claim 132, wherein the 6-pyruvoyltetrahydropterin synthase (PTPS) is at least 70% identical to SEQ ID NO: 41, more preferably at least 75% identical to SEQ ID NO; 41, more preferably at least 80% identical to SEQ ID NO: 41, more preferably at least 85% identical to SEQ ID NO: 41, more preferably at least 90% identical to SEQ ID NO: 41, more preferably at least 95% identical to SEQ ID NO: 41, more preferably at least 96% identical to SEQ ID NO: 41, more preferably at least 97% identical to SEQ ID NO: 41, more preferably at least 98% identical to SEQ ID NO: 41, more preferably at least 99% identical to SEQ ID NO: 41, more preferably 100% identical to SEQ ID NO: 41.

138. The expression system according to claim 132, wherein the biologically active fragment is the catalytic domain of tyrosine hydroxylase (SEQ ID NO: 12) and/or (SEQ ID NO: 40).

139. The expression system according to claim 132, wherein said biologically active variant is a mutated tyrosine hydroxylase polypeptide, wherein one or more of the residues S19, S31, S40 or S404 of SEQ ID NO: 7 have been altered to another amino acid residue.

140. The expression system according to claim 132, wherein the nucleotide sequence encoding a GTP-cyclohydrolase 1 (GCH1) polypeptide or a biologically active fragment or variant thereof comprises the sequence of SEQ ID NO: 20, or wherein said second nucleotide sequence encoding a tyrosine hydroxylase (TH) polypeptide or a biologically active fragment or variant thereof comprises a sequence selected from the group consisting of SEQ ID NO: 23, 24, 25, 26 and 27.

141. The expression system according to claim 132, wherein said first and said second and said third promoter are different or identical promoter sequences.

142. The expression system according to claim 132, wherein said promoter is an inducible promoter, optionally wherein said promoter is selected from the group consisting of Tet-On, Tet-Off, Mo-MLV-LTR, Mx1, progesterone, RU486 and/or Rapamycin-inducible promoter, optionally wherein the expression pattern of said promoter is regulated by a systemically administrable agent.

143. The expression system according to claim 132, wherein said expression system is a plasmid or naked plasmid DNA or plasmid DNA packaged within a vector.

144. The expression system according to claim 132, wherein said viral vector is selected from the group consisting of an adeno associated vector (AAV), lentiviral vector, adenoviral vector and retroviral vector.

145. The expression system according to claim 132, wherein the AAV vector is a self-complementary AAV (scAAV) vector, optionally wherein the nucleotide sequence encoding a tyrosine hydroxylase is a self-complementary sequence.

146. The expression system according to claim 132, further comprising one or more polyadenylation sequences or SV40 polyadenylation sequence.

147. The expression system according to claim 132, further comprising a post-transcriptional regulatory element, optionally wherein said post-transcriptional regulatory element is a Woodchuck hepatitis virus post-transcriptional regulatory element (WPRE).

148. The expression system according to claim 132, further comprising an intron wherein said intron is operably liked to the 5′ end of the TH and/or GCH-1 and/or PTPS transcript.

149. The expression system according to claim 132, wherein a fourth polynucleotide upon expression encodes a transport protein such as vesicular monoamine transporter (VMAT).

150. An isolated host cell transduced or transfected by the expression system of claim 132.

151. The host cell according to claim 150, wherein said cell is a stem cell.

152. A pharmaceutical composition for the treatment of Parkinson's disease and related conditions responding to 1-dopa treatment comprising the expression system of claim 132.

153. A method for reducing, delaying and/or preventing emergence of L-DOPA induced dyskinesia (LID), said method comprising peripherally administering the expression system of claim 132 to a patient in need thereof.

154. A method of obtaining and/or maintaining a therapeutically effective concentration of L-DOPA in blood, said method comprising peripherally administering the expression system of claim 132.

155. The method according to claim 154, further comprising administering an amount of tetrahydrobiopterin (BH₄) or an analogue thereof, and/or further comprising administering an amount of a peripheral decarboxylase inhibitor and/or COMT-inhibitor, optionally wherein the expression system, BH₄, decarboxylase inhibitor and/or COMT-inhibitor is administered by isolated limb perfusion.

156. The method according to claim 154, wherein the peripheral administration of the expression system is intramuscular administration or intravenous administration.

157. The method according to claim 54, for use in a method of treatment of a disease selected from the group consisting of Parkinson's Disease (PD); dyskinesia including L-DOPA induced dyskinesia (LID); DOPA responsive dystonia; ADHD; schizophrenia; depression; vascular parkinsonism; essential tremor; chronic stress; genetic dopamine receptor abnormalities; chronic opioid; cocaine; alcohol or marijuana use; adrenal insufficiency; hypertension; hypotension; noradrenaline deficiency; post-traumatic stress disorder; pathological gambling disorder; dementia; Lewy body dementia; hereditary tyrosine hydroxylase deficiency; atypical Parkinson's disease including conditions such as Multiple System Atrophy, Progressive Supranuclear Palsy, Vascular or arteriosclerotic Parkinson's disease, Drug induced Parkisonism and GTP cyclohydrolase 1 deficiency and/or any dystonic conditions due to dopamine deficiency.
